SEQUENCE LISTING (2 sequences)

SEQ ID NO: 1 - DNA, 77 bp, Artificial Sequence
  Synthetic primer
agtctagttactaggcagtgtagttagctgattgctaatagtaccaatcactaaccacacggccaggtaaaaagatt

SEQ ID NO: 2 - RNA, 23 nt, Artificial Sequence
  Synthetic primer
aggcaguguaguuagcugauugc

(19) United States
(12) Patent Application Publication
(10) Pub. No.: US 2014/0228423 A1
(43) Pub. Date: Aug. 14, 2014

(54) BIOMARKERS AND THERAPY FOR CANCER

(71) Applicants: Martin M. Matzuk, Houston, TX (US); Jaeyeon Kim, Houston, TX (US); Zhifeng Yu, Houston, TX (US)

(72) Inventors: Martin M. Matzuk, Houston, TX (US); Jaeyeon Kim, Houston, TX (US); Zhifeng Yu, Houston, TX (US)

(73) Assignee: BAYLOR COLLEGE OF MEDICINE, Houston, TX (US)

(21) Appl. No.: 14/122,669

(22) PCT Filed: Jun. 1, 2012
(86) PCT No.: PCT/US2012/040485

§ 371 Date: Mar. 31, 2014

Related U.S. Application Data
(60) Provisional application No. 61/492,082, filed on Jun. 1, 2011.

(51) Int. Cl. C12N 15/113; G01N 33/574; C12Q 1/68; A61K 31/497; A61K 31/439; A61K 31/7105

(52) U.S. Cl. CPC C12N 15/113; A61K 31/439; A61K 31/7105; C12Q 1/6886; A61K 31/497; G01N 33/57449
USPC 514/44 A; 506/9; 514/255.05; 435/7.23; 506/2; 435/7.92; 435/7.4

(57) ABSTRACT

In embodiments of the present invention, there are methods and compositions related to diagnosis and treatment of serous ovarian cancer. In specific embodiments, the invention encompasses methods related to miR-34c in diagnosis and treatment methods for serous ovarian cancer. In specific embodiments, the invention encompasses treatment methods for pancreatic cancer and other responsive cancers.

### CROSS-REFERENCE TO RELATED APPLICATIONS

This application claims priority from U.S. Provisional Patent Application Ser. No. 61/492,082 filed on Jun. 1, 2011, which is incorporated by reference herein in its entirety.

### STATEMENT REGARDING FEDERALLY SPONSORED RESEARCH OR DEVELOPMENT

This invention was made with government support under Grant Number CA060651 awarded by the National Cancer Institute. The government has certain rights in the invention.

### TECHNICAL FIELD

The present invention concerns at least the fields of molecular biology, cell biology, and medicine, including cancer medicine.

### BACKGROUND OF THE INVENTION

Ovarian cancer is the fifth most common cause of cancer-related death in women and the leading cause of death due to gynecologic malignancy (Cho and Shih, 2009; Bast et al., 2009). Despite advances in treatment, women with ovarian cancer have an unfortunate 5-year survival rate of only 46% (Jemal et al., 2011). Most ovarian cancers are histologically classified as high-grade serous adenocarcinomas, which are most commonly present at an advanced stage (Seidman et al., 2004) The cell of origin of serous carcinomas is unknown, and histologically similar cancers are present in the peritoneum, fallopian tube, and ovary (Bast et al., 2009; Kurman and Shih, 2010; Salvador et al., 2009; Ahmed et al., 2010). Recent studies suggest that low-grade serous ovarian cancers may originate from the surface epithelium of the ovary, whereas high-grade ovarian cancers originate in the fimbriated end of the fallopian tube and subsequently spread to the ovary and peritoneum (Cho and Shih, 2009; Kurman and Shih, 2010).

Pancreatic cancer is the fourth most lethal cancer in women and men, killing approximately 37,390 patients annually in the United States. Although there have been significant advances in the strategies to treat and cure many cancer patients, women with high-grade serous ovarian cancer (70% of ovarian cancers) continue to have an unfortunate 5-year survival rate of only 31%, while patients diagnosed with pancreatic cancer are even less fortunate, showing a meager 5-year survival rate of only 5.8%.

MicroRNAs (miRNAs), 20-25-nucleotide non-coding RNAs, have emerged as critical regulators of cancer development (Ventura and Jacks, 2009), and expression of miRNAs is altered in many human cancers including ovarian cancer (Calin et al., 2004; Iorio et al., 2007; Zhang et al., 2008). High levels of the miRNA biosynthesis enzymes, DICER and DROSHA, correlate with increased survival for ovarian cancer patients (Merritt et al., 2008), and low levels of DICER in ovarian cancers have been observed in one study (Pampalakis et al., 2009) but not in another (Flavin et al., 2008). Alternatively, mutations that activate the PI3K/AKT/mTOR pathway are observed in 70% of ovarian cancers and are linked to chemotherapeutic resistance (Bast et al., 2009). Reduction of all miRNAs through shRNA suppression of DICER promotes tumorigenesis a Kras-mutant lung cancer model (Kumar et al., 2007). By understanding the origin and genetics of ovarian cancer, one can develop more effective diagnostic and therapeutic strategies.

### BRIEF SUMMARY OF THE INVENTION

The present invention is directed to methods and compositions that concern at least cancer diagnosis, risk for developing cancer, and cancer therapeutics. In certain cases the cancer may be of any type, although in specific embodiments the cancer is serous ovarian cancer. An individual that is provided with methods and/or compositions of the invention may be a mammal, including human, dog, horse, or cat, for example.

In certain embodiments the present invention concerns the identification of novel microRNAs and biomarkers for serous ovarian cancer.

In some embodiments of the invention, there is a method of treating serous ovarian cancer in an individual, comprising the step of delivering to the individual a therapeutically effective amount of miR-34c or a miR-34c mimic The mature sequence of miR-34c is AGGCAGUGUAGUUAGCUGAUUGC (SEQ ID NO:2) and may be used, in specific embodiments. If necessary, a Taqman QPCR assay would be performed to quantitate the levels of miR-34c being delivered. In some aspects of the invention, the individual is provided at least one other treatment for serous ovarian cancer, such as treatment that comprises an inhibitor of survivin, mTOR inhibitor, withaferin A, parthenolide, vorinostat, scriptaid, or a combination thereof. In specific embodiments, the inhibitor of survivin is YM155 or a nucleic acid (such as shRNA) that targets survivin. In some cases, the mTOR inhibitor is everolimus.

In particular embodiments of the invention, the cancer is identified as having upregulation of leukocyte-associated Ig-like receptor 1; lymphocyte antigen 6 complex, locus E; and/or tumor necrosis factor receptor, member 21. In certain cases, the cancer is identified as having upregulation of a gene selected from the group consisting of phosphoprotein 1, musin 16(CA125), folate receptor 1, chemokine (C-X-X motif) ligand 9, chemokine (C-X-X motif) ligand 10, chemokine (C-X-X motif) ligand 8, cytokeratin 14, cytokeratin 8, cytokeratin 17, and a combination thereof.

In some embodiments of the invention, the serous ovarian cancer originates in the fallopian tube.

In some embodiments, there is a method of determining whether an individual has serous ovarian cancer or is at risk for developing serous ovarian cancer, comprising the step of determining that when the expression of one or more of leukocyte-associated Ig-like receptor 1; lymphocyte antigen 6 complex, locus E; and/or tumor necrosis factor receptor, member 21 is upregulated in a sample from the individual, the individual has serous ovarian cancer or is at risk for having serous ovarian cancer.

An individual may be considered at risk for serous ovarian cancer if she has a personal or family history of breast, ovarian, endometrial, prostate, or colon cancer, especially if her mother or sister had ovarian cancer. Other risk factors include age, use of high-dose estrogen for long periods without progesterone, r uninterrupted ovulation due to infertility, no pregnancies, no use of birth control, being Jewish, or having a defect in the BRCA1 or BRCA2 gene. Diagnostic methods other than those employed herein may be utilized, such as a pelvic examination, rectovaginal examination, transvaginal ultrasound, and/or a tumor marker blood test for CA-125, for example.

In some embodiments, an agent for cancer therapy is used that comprises an oligonucleotide that functions via RNA interference. In some embodiments, the oligonucleotide is an antisense oligonucleotide, an siRNA, an shRNA, an miRNA or related molecules, or combinations thereof.

In certain embodiments of the invention, there is a method of treating cancer with a combination of YM155 and parthenolide. In some aspects of the invention, cancers are treated with a combination of an agent that suppresses HSPA1A and HSPA1B and increases endoplasmic reticulum stress (e.g., YM155) and an agent that increases reactive oxygen species (e.g., parthenolide) or suppresses anti-oxidants.

In specific embodiments, the invention encompasses methods and/or compositions of using miRNA or combination treatments for ovarian cancer for treatment of other cancers such as pancreatic cancer, for example.

In some embodiments, there is a method of treating serous ovarian cancer in an individual, comprising the step of delivering to the individual a therapeutically effective amount of miR-34c or a miR-34c mimic, and in some cases the individual is provided at least one other treatment for serous ovarian cancer, such as an inhibitor of survivin, an inhibitor or suppressor of HSPA1A and HSPA1B, a stimulator of endoplasmic reticulum stress, an mTOR inhibitor, withaferin A, parthenolide, piper longumine, vorinostat, scriptaid, camptothecin, small molecules that generate or enhance reactive oxygen species inhibitors of the anti-oxidant system, or a combination thereof. In specific embodiments, the inhibitor of survivin is YM155 or an siRNA or shRNA that targets surviving and/or the mTOR inhibitor is everolimus. In certain cases, the small molecule that generates or enhances reactive oxygen species is Motexafingadolium, Eleggclomol, parthenolide, piper longumine, dimethylamino-parthenolide, or costunolide, for example. In certain cases, the inhibitor of the anti-oxidant system is Buthionine, sulphoximine, Imexon, Mangafodipir, 2-methoxyestradiol, or Tetrathiomolybdate. In specific embodiments, the inhibitor or suppressor of HSPA1A and HSPA1B is YM155 or an shRNA or siRNA that targets HSPA1A and HSPA1B. In some embodiments, the combination therapy to treat ovarian cancer, pancreatic cancer, and other responsive cancers involves delivery of YM155 and parthenolide.

In some embodiments of the invention, the combination therapy to treat ovarian cancer, pancreatic cancer, and other responsive cancers involves delivery of a small molecule that enhances endoplasmic reticulum stress and generates or enhances reactive oxygen species or suppresses the anti-oxidant system. In a specific embodiment, the small molecule that enhances endoplasmic reticulum stress is YM155. In some cases, the small molecule that generates or enhances reactive oxygen species is parthenolide, dimethyl-parthenolide, or costunolide.

In particular embodiments, the inhibitor of the anti-oxidant system is Buthionine, sulphoximine, Imexon, Mangafodipir, 2-methoxyestradiol, or Tetrathiomolybdate.

In some embodiments of the invention, a cancer is identified as having upregulation of a gene selected from the group consisting of Secreted phosphoprotein 1, Chemokine (C-X-C motif) ligand 9, Chemokine (C-X-C motif) ligand 10, CD72 antigen, Solute carrier family 15, member 3, CD84 antigen, Complement component 1qB, Plasminogen activator, urokinase, Lymphocyte antigen 86, Mucin 16 (CA125), Folate receptor 1, Solute carrier family 11, member 1, Solute carrier family 12, member 8, CD40 antigen, Immunoglobulin superfamily, member 9, Interleukin 10 receptor, alpha, Tumor necrosis factor receptor, member 12a, Apolipoprotein E, Toll-like receptor 7, Transmembrane protein 48, Interleukin 1 receptor, type II, Leukocyte-associated Ig-like receptor 1, Lymphocyte antigen 6 complex, locus E, A disintegrin and metallopeptidase domain 17, Pleiotrophin, CD83 antigen, Chemokine (C-C motif) ligand 8, Transmembrane channel-like gene family 6, Transmembrane protein 49, Endothelial cell-specific molecule 1, Anti-Mullerian hormone type 2 receptor, Midkine, Transmembrane protein 173, Tumor necrosis factor receptor, member 21, Complement factor B, Secretory carrier membrane protein 5, and a combination thereof.

In some embodiments of the invention, a cancer originates in the fallopian tube.

In some embodiments, there is a method of determining whether an individual has serous ovarian cancer or is at risk for developing serous ovarian cancer, comprising the step of determining that when the expression of one or more of Secreted phosphoprotein 1, Chemokine (C-X-C motif) ligand 9, Chemokine (C-X-C motif) ligand 10, CD72 antigen, Solute carrier family 15, member 3, CD84 antigen, Complement component 1qB, Plasminogen activator, urokinase, Lymphocyte antigen 86, Mucin 16 (CA125), Folate receptor 1, Solute carrier family 11, member 1, Solute carrier family 12, member 8, CD40 antigen, Immunoglobulin superfamily, member 9, Interleukin 10 receptor, alpha, Tumor necrosis factor receptor, member 12a, Apolipoprotein E, Toll-like receptor 7, Transmembrane protein 48, Interleukin 1 receptor, type II, Leukocyte-associated Ig-like receptor 1, Lymphocyte antigen 6 complex, locus E, A disintegrin and metallopeptidase domain 17, Pleiotrophin, CD83 antigen, Chemokine (C-C motif) ligand 8, Transmembrane channel-like gene family 6, Transmembrane protein 49, Endothelial cell-specific molecule 1, Anti-Mullerian hormone type 2 receptor, Midkine, Transmembrane protein 173, Tumor necrosis factor receptor, member 21, Complement factor B, or Secretory carrier membrane protein 5 is upregulated in a sample from the individual, the individual has serous ovarian cancer or is at risk for having serous ovarian cancer.

In particular embodiments, there is a method of treating cancer in an individual, comprising the step of delivering a therapeutically effective amount of YM155 and parthenolide to the individual. In specific cases, the individual has ovarian cancer or pancreatic cancer.

Although in specific embodiments of the invention there are methods and compositions for ovarian and/or pancreatic cancer, in some cases they are applicable to any other cancer, including brain, breast, prostate, colon, skin, lung, testicular, cervical, liver, spleen, gall bladder, thyroid, esophageal, head and neck, blood, rectal, and so forth.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1. Dicer-Pten DKO mice develop high-grade metastatic serous carcinoma. (A) Severe ascites in an 8.4-month-old DKO mouse. (B) Survival curve of DKO and control mice. (C) Bilateral ovarian/fallopian tube tumors are observed in a DKO mouse at 6 months. (D) The DKO mouse described in (A) showing extensive peritoneal metastasis with clusters of tumor nodules (yellow arrows) and a massive accumulation on the diaphragm (green arrows). (E) Histological analyses of DKO ovarian tumors showing papillary structure and slit-like spaces (H&E, 10×). (F) Ovarian tumor with a solid growth pattern, and high-grade nuclear features including nuclear pleomorphism (red arrow), prominent nucleoli (green arrow), apoptosis (black arrows), and brisk mitotic activity (yellow arrow) (H&E, 20×). (G) A peritoneal metastatic cancer displaying solid growth and high-grade nuclear features as seen in the ovarian tumor (H&E, 40×). (H) Similar high-grade serous carcinomas in a NOD SCID mouse that was injected with ascites cells from a DKO mouse (H&E, 40×).

FIG. 2. Fallopian tube is the origin of high-grade serous carcinoma in Dicer-Pten DKO mice. (A) Early tumors form in the fallopian tube (yellow arrows) of a 5-month-old DKO mouse with normal ovaries (white arrowheads). (B) Progression of the fallopian tube tumors in a DKO mouse at 8 months. Ovaries are still intact (white arrowheads). (C) Tumor (black arrowhead) in an 8-month-old DKO mouse after ipsilateral removal of the ovary. (D) No tumor in the ovary (white arrowhead) in an 8-month-old DKO mouse with the fallopian tube removed ipsilaterally. (E) Histological analyses of an early fallopian tube lesion, in which tumor cells extensively infiltrate and expand the stroma of the fallopian tube (H&E, 4×). (F, G) Abundant immunohistochemical staining of cytokeratin 14 (KRT14) (F, 4×) and cytokeratin 8 (KRT8) (G, 4×) in proliferating tumor cells. (H) Neoplastic cells are primarily located within the stroma with an overlying benign-looking tubal epithelium (H&E, 20× magnification of a region from panel E). (I) KRT14-positive tumor cells focally invade and erode the fallopian tube epithelium (20× magnification of a region from panel F). (J) Massively proliferating tumor cells show abundant Ki67 expression with no significant expression in fallopian tube epithelium (10×). (K-M) Low- (10×; K) and high-magnification (20×; L, M) images of a very early fallopian tube lesion. A small nest (long arrow) and a few single tumor cells (short arrow) showing a strong KRT14 (K, L) and KRT17 (M) expression, compared with the fallopian tube epithelium (arrowheads) and uninvolved stroma that are KRT14-negative (K, L).

FIG. 3. Activation of the PI3K pathway in Dicer-Pten DKO mice. Western blot analysis of DKO fallopian tube tumors showing activation of AKT signaling compared with control fallopian tubes, as indicated by the enhanced expression of phosphorylated AKT, phosphorylated PRAS40, phosphorylated 4E-BP1, survivin, and stathmin.

FIG. 4. Activation of the PI3K pathway in Dicer/Pten DKO mice and effects of mir34c and everolimus on a Dicer/Pten DKO cell line. A, Western blot analysis of DKO fallopian tube tumors showing activation of AKT signaling compared with control fallopian tubes. B, A Dicer/Pten DKO cell line was transfected with miR-34c mimic or a control (miR-Ctrl), treated with everolimus (0.8 uM) or without (DMSO), and examined 48 h later for the effects on cell viability.

FIG. 5. Model for the interactions of the PI3K/AKT/mTOR and miRNA pathways in high-grade serous carcinoma. Based on data with the Dicer/Pten DKO, the inventors considered that activation of the PI3K/AKT/mTOR pathway and absence of miR-34c, a downstream target of p53, leads to growth and transformation of mesenchymal cells in the fallopian tube to epithelial cancer cells. Factors in green are oncogenic, and factors in red are tumor suppressors of the pathway. Black arrows, known positive regulation; blue arrows, additional regulation; red blunted-end lines, negative regulation. LY294002, everolimus, and PP242 are small molecule inhibitors of the PI3K/AKT/mTOR pathway.

FIG. 6. Genetic perturbations in human high-grade serous carcinomas (A), expression of miRNAs in mouse fallopian tube (B), and levels of miR34-c in human high-grade serous carcinomas (C). A. DNA copy number changes in the PTEN, DICER, and MIR34B/C loci in human serous ovarian cancers. B. Fallopian tube expression of the most abundant miRNAs using next generation sequencing.

FIG. 7. Model for the synergistic relationship of the PI3K, p53, and miR-34c pathways in serous carcinoma. Receptor tyrosine kinases relay signals through the PI3K pathway to stimulate growth, proliferation, and survival and block p53-mediated apoptosis. miR-34c is positively regulated by p53 and directly suppresses CDCA8, MCM5, and CCNE2. Treatment of mouse and human high-grade serous ovarian cancer cells with a miR-34c mimic causes apoptosis, a block in DNA replication, and failure to progress through the G1 phase of the cell cycle, while the survivin inhibitor, YM155, causes apoptosis of ovarian cancer cells at nanomolar concentrations.

FIG. 8. Analysis of DICER/PTEN DKO mice and tumors. A. DICER/PTEN DKO mice initially develop fallopian tube cancers (yellow arrows) with no involvement of the ovaries (white arrowheads). B. Unilateral removal of the ovary does not prevent serous adenocarcinoma formation (black arrow). C. Unilateral removal of the fallopian tube prevents tumors from forming and engulfing the ovary (white arrow). D, E. Ascites is observed in a mouse with metastatic spread of the primary cancer (black arrows) to the peritoneum overlying the diaphragm (green arrows) and other sites (yellow arrows). F. Survival curves for DICER/PTEN DKO mice compared to controls. G. High-grade serous carcinoma with focal papillary architecture consisting of small and incomplete papillae with slit-like fenestrations. The neoplastic cells are characterized by enlarged pleomorphic nuclei (green arrow) with prominent nucleoli (red arrow) and frequent mitoses (yellow arrow). H. High-grade serous carcinoma with a more solid growth pattern and high-grade nuclear features including nuclear pleomorphism, prominent nucleoli, apoptosis (black arrow), and brisk mitotic activity. I, J. Arrows indicate early fallopian tube serous cancers that are moderately positive for CA125 and strongly positive for cytokeratin 17 (KRT17). The epithelium lining the fallopian tube lumen in both panels is weakly positive for both markers.

FIG. 9. Activation of the PI3K pathway in PTEN/DICER DKO mice. Western blot analysis of DKO fallopian tube tumors showing activation of PI3K signaling compared with control fallopian tubes as indicated by the enhanced expression of phosphorylated (P)-AKT, P—PRAS40, P-4EBP1, survivin, and stathmin. On the right side of the figure, mRNA enrichment in the mouse primary serous cancers versus control fallopian tubes is presented.

FIG. 10. Drug and miRNA mimic effects on ovarian cancer cell viability (A-C) and proliferation (D). OVCAR8 cancer cells were incubated for 48 hours in the presence of YM155 (A) or everolimus (B) and assayed for cell viability. C. Three independent PTEN/DICER DKO mouse serous carcinoma cell lines were transfected with miRNA control (miR-Ctrl) or miR-34c mimics and assayed for cell viability 48 hours later. D. OVCAR8 cells were infected with lentivirus expressing miR-Ctrl or miR-34c and assayed for cell number. *, P<0.05; **, P<0.005; ***, P<0.0005

FIG. 11. Human high-grade serous ovarian cancer cells phenocopy the normal spread of serous carcinomas. A, E. Human cancer cells home in to the mouse ovaries, proliferate, and develop into large human ovarian cancer masses around the mouse ovaries. B, C. The cancer cells from the large tumor in panel A proliferate in clusters around the ovary (0v), are observed within the bursa (arrow), are keratin 8-positive (C), and are surrounded by keratin 8-negative reactive stroma. D. The ovarian cancers (blue arrows) also have a predilection for invading the diaphragm. F, G. Histology of the large tumor in panel E demonstrates sheets of neoplastic cells with pleomorphic nuclei with prominent nucleoli (green arrow) and high mitotic activity (yellow arrow).

FIG. 12. miRNA mimic initial screening reveals the significance of miR-34c in Dicer-Pten DKO mouse ovarian cancer.

FIG. 13. Validated cell viability inhibitory effect of miR-34c (A) in Dicer-Pten DKO mouse ovarian cancer cells is associated with cell cycle arrest in G1 phase (B).

FIG. 14. miR-34c inhibits cyclinE-CDK2 complex by down regulating CDK2 (A), cyclinE (B) and up regulating CDKN1C(C) in Dicer-Pten DKO mouse ovarian cancer cells.

FIG. 15. miR-34c levels were decreased 83-fold in human serous adenocarcinomas compared with fallopian tube by a Taqmand QPCR assay.

FIG. 16. Similar effect of miR-34c in human serous ovarian cancer cell proliferation (A) by arresting cancer cells in G1 phase (B).

FIG. 17. Dose response curve of YM155 after 48-hr treatment of OVCAR8 cells. GI50=4.6 nM

FIG. 18. HSPA1A/1B (A, B) and BIRC5 (C) knockdown in OVCAR8. QPCR analysis of gene expression (A) and relative cell number after lentiviral shRNA control (Ctrl) or combined HSPA1A and HSPA1B knockdown (B) or BIRC5 knockdown (C). Inset: Survivin protein levels were reduced compared to control shRNA.

FIG. 19. Synergistic effect of YM155 and parthenolide in inhibiting the growth of ovarian cancer cell lines (A and B) and a pancreatic cancer cell line (C). Cells were treated as shown for 48 hours, and cell viability was determined using the Cell Titer Glo assay. When levels of YM155 or parthenolide alone inhibited ˜50% of cancer cell viability, the combination of these two compounds inhibited nearly >90% of cancer cells. The findings indicate synergy of these two compounds.

FIG. 20. High-throughput screen for drugs that synergize with YM155. Cell viability of YM155 at 3 nM was 64.3%+/−2.8%. Digitonin (0.8 mg/ml) was used as a positive control for ˜100% cell death. Average “synergy” of the 1120 compounds +/−YM155 is 1.1 (mean)+/−7.7 (S.D.). The relative synergies in a single-dose combinatorial drug screen were determined based on the equation published by Lundberg (1997) where expected theoretical additive values were calculated according to the equation, c=a×b/100, in which a and b are cell survival values after single agent treatment, given as percent of vehicle treated control. The relative synergy for each drug was then calculated by subtracting measured cell survival of combinatorial treatment from the calculated c value.

### DETAILED DESCRIPTION OF THE INVENTION

### I. Definitions

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the RNA molecule processed from a precursor.

The term “miRNA mimic” as used herein refers to one or more nonnatural double-stranded miRNA-like RNA fragments. Such an RNA fragment is designed to have its 5′-end bearing a partially complementary motif to the selected sequence in the 3′UTR unique to the target gene. Once introduced into cells, this RNA fragment, mimicking an endogenous miRNA, can bind specifically to its target gene and produce posttranscriptional repression, more specifically translational inhibition, of the gene. Unlike endogenous miRNAs, miR-Mimics act in a gene-specific fashion.

### II. General Embodiments of the Invention

The cell of origin of serous ovarian cancer is unknown. To generate a mouse model for this lethal cancer and identify early cancer biomarkers, the inventors conditionally deleted both Dicer (essential for microRNA biosynthesis) and Pten (a negative regulator of the PI3K pathway) in the female reproductive tract. Beginning at ˜3-5 months, these Dicer/Pten mutant mice develop high-grade serous carcinomas that initiate in the stroma of the fallopian tube through a mesenchymal-to-epithelial transition (MET), subsequently envelop the ovary, and then metastasize throughout the peritoneum, resulting in ascites and 100% lethality by 13 months. The fallopian tube cancers demonstrate upregulation of genes encoding known and novel secreted proteins that are biomarkers. This invention uncovers a new paradigm for the initiation of high-grade serous ovarian cancer.

Ovarian cancer is the leading cause of death due to gynecologic malignancy, annually affecting ˜22,000 U.S. women of all ethnic backgrounds. Although there have been significant advances in the strategies to treat and cure many cancer patients, women with high-grade serous ovarian cancer continue to have an unfortunate 5-year survival rate of only 31%. To increase the survival rate of these women afflicted with this deadly disease, better diagnostic strategies must be developed and innovative treatment approaches must be adopted. This invention concerns the development of new tools, resources, and strategies to understand the pathogenesis of high-grade serous carcinoma, the most common histologic type of ovarian cancer. Using a mouse genetics approach, the inventors created a mouse model in which they deleted in the female reproductive tract both DICER, an essential enzyme involved in the production of mature microRNAs, and PTEN, the tumor suppressor that inhibits the PI3K/AKT/mTOR pathway. Consistent with recent theories that high-grade serous ovarian cancer arises in the fallopian tube, it was discovered that the DICER/PTEN double knockout mice develop serous carcinomas of the fallopian tube that quickly engulf the ovary and then metastasize to the peritoneum, resulting in ascites and 100% lethality by 13 months. By analysis of the gene expression in the early fallopian tube cancers, multiple upregulated genes that encode secreted and transmembrane proteins were uncovered that are early biomarkers of this common and lethal cancer in women, in certain embodiments of the invention. Also, miRNAs are characterized that are tumor suppressors, in certain aspects of the invention, and their mechanism of action and drugs that are useful to treat high-grade serous carcinoma in the clinic are encompassed herein. Embodiments of the invention include the DICER/PTEN double knockout mouse model to investigate the pathogenesis of high-grade serous carcinoma development in vivo and the mechanisms by which these cancer cells metastasize. To develop better therapeutics and diagnostics for these cancers, next generation sequencing and gene expression tools are employed to identify unique mutations, altered pathways, and novel biomarkers in these high-grade serous carcinomas. The in vivo cancer-prone DICER/PTEN double knockout model and cancer cell lines derived from these mice and additional mouse models and human serous cancer cells lines are used to characterize the findings for uncovering novel therapeutic approaches for treating women at different stages of their cancer.

In specific embodiments, miR-34c is employed in methods and compositions of the invention, and the sequence for human precursor miR-34c stem-loop is AGTCTAGTTA CTAGGCAGTG TAGTTAGCTG ATTGCTAATA GTACCAATCA CTAACCACAC GGCCAGGTAA AAAGATT (SEQ ID NO:1; GenBank® Accession No. NR_{—}029840). The mature sequence of miR-34c is AGGCAGUGUAGUUAGCUGAUUGC (SEQ ID NO:2).

The present invention is directed to compositions and methods relating to preparation and characterization of miRNAs, as well as use of miRNAs for therapeutic, prognostic, and diagnostic applications.

### III. miRNA Molecules

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved by an enzyme called Dicer in animals Dicer is ribonuclease III-like nuclease. The processed miRNA is typically a portion of the stem.

The processed miRNA (also referred to as “mature miRNA”) become part of a large complex to down-regulate a particular target gene. Examples of animal miRNAs include those that imperfectly basepair with the target, which halts translation (Olsen et al., 1999; Seggerson et al., 2002). SiRNA molecules also are processed by Dicer, but from a long, double-stranded RNA molecule. SiRNAs are not naturally found in animal cells, but they can function in such cells in a RNA-induced silencing complex (RISC) to direct the sequence-specific cleavage of an mRNA target (Denli et al., 2003).

A. Nucleic Acids

The present invention concerns miRNAs that can be labeled, used in array analysis, or employed in diagnostic, therapeutic, or prognostic applications. The RNA may have been endogenously produced by a cell, or been synthesized or produced chemically or recombinantly. They may be isolated and/or purified. The term “miRNA,” unless otherwise indicated, refers to the processed RNA, after it has been cleaved from its precursor. Table 1 indicates which SEQ ID NO corresponds to the particular precursor sequence of an miRNA and what sequences within the SEQ ID NO conespond to the mature sequence. The name of the miRNA is often abbreviated and referred to without the prefix and will be understood as such, depending on the context. Unless otherwise indicated, miRNAs referred to in the application are human sequences identified as mir-X or let-X, where X is a number and/or letter.

In certain experiments, an miRNA probe designated by a suffix “5P” or “3P” can be used. “5P” indicates that the mature miRNA derives from the 5′ end of the precursor and a corresponding “3P” indicates that it ferives from the 3′ end of the precursor, as described on the world wide web at sanger.ac.uk/cgi-bin/rfam/mlrna. Moreover, in some embodiments, an miRNA probe is used that does not conespond to a known human miRNA. It is contemplated that these non-human miRNA probes may be used in embodiments of the invention or that there may exist a human miRNA that is homologous to the non-human miRNA. While the invention is not limited to human miRNA, in certain embodiments, miRNA from human cells or a human biological sample is evaluated. In other embodiments, any mammalian cell or biological sample may be employed.

In some embodiments of the invention, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, control nucleic acids, and other probes and primers. In many embodiments, miRNA are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or between an miRNA and its target are such lengths. Moreover, the complementarity may be expressed as a percentage, meaning that the complementarity between a miRNA or mimic and its target is 90% or greater over the length of the miRNA or mimic.

It is understood that a miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The term “recombinant” may be used and this generally refers to a molecule that has been manipulated in vitro or that is the replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. miRNA probes of the invention can be or be at least 60, 65, 70, 75, 80, 85, 90, 95, or 100% complementary to their target.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

1. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

2. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the l′-position of a 5-carbon sugar. In another non-limiting example, a nucleoside comprising a pyrimidine nucleobase (i.e., C, T or U) typically covalently attaches a 1 position of a pyrimidine to a l′-position of a 5-carbon sugar (Kornberg and Baker, 1992).

3. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

4. Nucleic Acid Analogs

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acids probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moiety which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake and regulating RNA expression; U.S. Pat. No. 5,858,988, which describes hydrophobic carrier agent attached to the 2′-O position of oligonucleotides to enhanced their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and 5,480,980 (7-deaza-2′ deoxyguanosine nucleotides and nucleic acid analogs thereof).

5. Modified Nucleotides

Labeling methods and kits of the invention specifically contemplate the use of nucleotides that are both modified for attachment of a label and can be incorporated into an miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, and NEN. Functional groups may be prepared according to ways known to those of skill in the art, including the representative information found in U.S. Pat. Nos. 4,404,289; 4,405,711; 4,337,063 and 5,268,486, and Br. Pat. No. 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N⁶-(4-amino)butyl-ATP, N⁶-(6-amino)butyl-ATP, N⁴-[2,2-oxy-bis-(ethylamine)]-CTP; N⁶-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N⁶-(4-amino)butyl-dATP, N⁶-(6-amino)butyl-dATP, N⁴-[2,2-oxy-bis-(ethylamine)]-dCTP; N⁶-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

B. Preparation of Nucleic Acids

A nucleic acid may be made by any technique known to one of ordinary skill in the art, such as for example, chemical synthesis, enzymatic production or biological production. It is specifically contemplated that miRNA probes of the invention are chemically synthesized.

In some embodiments of the invention, miRNAs are recovered from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent, as described in Example 1.

Alternatively, nucleic acid synthesis is performed according to standard methods. See, for example, Itakura and Riggs (1980). Additionally, U.S. Pat. No. 4,704,362, U.S. Pat. No. 5,221,619, and U.S. Pat. No. 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

Diester Method.

The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester Method.

The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide Phosphorylase Method.

This is an enzymatic method of DNA synthesis that can be used to synthesize many useful oligonucleotides (Gillam et al., 1978; Gillam et al., 1979). Under controlled conditions, polynucleotide phosphorylase adds predominantly a single nucleotide to a short oligonucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-Phase Methods.

Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic nucleic acid synthesizers.

Phosphoramidite chemistry (Beaucage and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

Recombinant Methods.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

In certain embodiments, the present invention concerns nucleic acid molecules that are not synthetic. In some embodiments, the nucleic acid molecule has a chemical structure of a naturally occurring nucleic acid and a sequence of a naturally occurring nucleic acid, such as the exact and entire sequence of a single stranded primary miRNA (see Lee 2002), a single-stranded precursor miRNA, or a single-stranded mature miRNA. In addition to the use of recombinant technology, such non-synthetic nucleic acids may be generated chemically, such as by employing technology used for creating oligonucleotides.

C. Isolation of Nucleic Acids

Nucleic acids may be isolated using techniques well known to those of skill in the art, though in particular embodiments, methods for isolating small nucleic acid molecules and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lys ate and applying the alcohol/lysate mixture to a solid support before eluting the RNA molecules from the solid support. In some embodiments, the amount of alcohol added to a cell lys ate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried down and resuspended in a liquid and volume appropriate for subsequent manipulation.

### IV. miR-34c Mimics

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., pri- or pre-miRNAs). miRNA mimics can be comprised of modified or unmodified RNA, DNA, RNA-DNA hybrids, or alternative nucleic acid chemistries (e.g., LNAs or 2′-0,4′-C-ethylene-bridged nucleic acids (ENA)). For mature, double stranded miRNA mimics, the length of the duplex region can vary between 16 and 31 nucleotides and chemical modification patterns can comprise one or more of the following: the sense strand contains 2′-O-methyl modifications of nucleotides 1 and 2 (counting from the 5′ end of the sense oligonucleotide), and all of the Cs and Us. The antisense strand modifications comprise 2′ F modification of all of the Cs and Us, phosphorylation of the 5′ end of the oligonucleotide, and stabilized internucleotide linkages associated with a 2 nucleotide 3′ overhang. Mimics can also comprise linker conjugate modifications that enhance stability, delivery, specificity, functionality, or strand usage. Preferred microRNA mimics of the disclosure are duplexes formed between a sense strand and an antisense strand where the antisense strand has significant levels of complementarity to both the sense strand and to a target gene, and where:

a. the sense strand ranges in size from about 16 to about 31 nucleotides and nucleotides 1 and 2 (counting from the 5′ end) and all C nucleotides and all U nucleotides in the sense strand are 2′O-methyl modified;

b. the antisense strand ranges in size from about 16 to about 31 nucleotides and all C nucleotides and all U nucleotides in the antisense strand are 2′ F modified;

c. a cholesterol molecule is attached to the 3′ end of the sense strand via a C5 linker molecule such that the sense stand has the following structure (where “oligo” represents the nucleotides of the sense strand):

d. a phosphate group is present at the 5′ end of the antisense strand;

e. a 2 nucleotide overhang is present at the 3′ end of the antisense strand comprising phosphorothioate linkages; and

f. a mismatch is present between nucleotide 1 on the antisense strand and the opposite nucleotide on the sense strand and/or a mismatch is present between nucleotide 7 on the antisense strand and the opposite nucleotide on the sense strand and/or a mismatch is present between nucleotide 14 on the antisense strand and the opposite nucleotide on the sense strand (where the specified nucleotide positions are counted from the 5′ end of the antisense strand).

An “miRNA mimic” is an agent used to increase the expression and/or function of a miRNA. The miRNA mimic can also increase, supplement, or replace the function of a natural miRNA. In one embodiment, the miRNA mimic may be a polynucleotide comprising the mature miRNA sequence. In another embodiment, the miRNA mimic may be a polynucleotide comprising the pri-miRNA or pre-miRNA sequence. The miRNA mimic may contain chemical modifications, such as locked nucleic acids, peptide nucleic acids, sugar modifications, such as 2′-O-alkyl (e.g. 2′-O-methyl, 2′-β-methoxyethyl), 2′-fluoro, and 4′ thio modifications, and backbone modifications, such as one or more phosphorothioate, morpholino, or phosphonocarboxylate linkages. Certain miRNA mimics are commercially available from companies, such as Dharmacon (Lafayette, Colo.) and Ambion, Inc.

In some embodiments, the miRNA mimic may be expressed in vivo from vectors. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, an expression vector for expressing the miRNA mimic comprises a promoter “operably linked” to a polynucleotide encoding the particular miRNA. The phrase “operably linked” or “under transcriptional control” as used herein means that the promoter is in the correct location and orientation in relation to a polynucleotide to control the initiation of transcription by RNA polymerase and expression of the polynucleotide. The polynucleotide encoding the miRNA may encode the primary-microRNA sequence (pri-miRNA), the precursor-microRNA sequence (pre-miRNA) or the mature miRNA sequence. In a particular embodiment, the polynucleotide comprises the sequence of SEQ ID NO: 1. The polynucleotide encoding the particular miRNA may be about 18 to about 2000 nucleotides in length, about 70 to about 200 nucleotides in length, about 20 to about 50 nucleotides in length, or about 18 to about 25 nucleotides in length. In other embodiments, the polynucleotide encoding the particular miRNA is located in a nucleic acid encoding an intron or in a nucleic acid encoding an untranslated region of an mRNA or in a non-coding RNA.

In some embodiments of the invention, a miR-34c mimic is utilized that is no more than 100, 95, 90, 85, 80, or 77 nt in length, for example. The mimic may be substantially identical to SEQ ID NO:1 or 2, in certain embodiments. In specific embodiments, the miR-34c mimic is at least 80%, 85%, 90%, 95%, 97%, or 99% identical to SEQ ID NO:1 or 2.

### V. Combination Treatments

In some embodiments of the invention, it may be desirable to combine the compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes one agent and the other includes the second agent(s).

Cancer chemotherapy began in the 1940's with the use of nitrogen mustard and antifolates to treat lymphomas and leukemias, respectively. In the 1960's, combination therapy was first used and has become the mainstay of most successful chemotherapeutic regimens today. While many forms of cancer have responded well to targeted and non-specific chemotherapeutic regimes, most patients continue to die from their cancers or side effects of the chemotherapy.

A variety of chemotherapeutic agents have been and are continuing to be used in the clinic to treat cancers. Whereas some of these anti-cancer drugs have been designed to inhibit specific protein targets (e.g., Gleevec for cancers carrying the BCR-ABL fusion protein), many of the drugs unfortunately act non-specifically. For example, drugs that kill the highly proliferating cancer cells will also kill actively dividing normal cells in the gastrointestinal tract and bone marrow, thereby producing unwanted side effects of nausea, anemia, and infections. In a few cases, drugs that have been shown to target one cellular pathway in vitro act through off target effects on another protein or pathway. The inventors have uncovered one such anti-cancer drug that does not kill cancer cells through the discovered route but instead acts through another major pathway. In addition, the inventors discovered an additional drug that is modestly effective at killing cancer cells when used alone and for which its cytotoxic pathway is still unknown. However, when cancer cells are treated simultaneously with these two drugs, there is a synergistic effect with dramatically increased cell death. In embodiments of the invention, one can employ the methods and/or compositions on pancreatic cancer and serous ovarian cancer, two deadly and common cancers with poor 5-year survival rates of 6% and 31%, respectively. Whereas progress has been made in many cancers, the cure rates for these cancers have changed little in three decades. One can also identify potent drugs and drug combinations that will not just lengthen survival times but will markedly improve the cure rates for these deadly cancers.

Tumor cell resistance to chemotherapy and radiotherapy agents, for example, represents a major problem in clinical oncology. One goal of current cancer research is to find ways to improve the efficacy of some cancer therapies by also employing others. In the context of the present invention, it is contemplated that the present invention could be used similarly in conjunction with chemotherapeutic, radiotherapeutic, or immunotherapeutic intervention, for example.

Alternatively, the present invention may precede or follow the other agent treatment by intervals ranging from minutes to weeks. In embodiments where the other agent and inventive treatment are applied separately to the cell, one would generally ensure that a significant period of time did not expire between the time of each delivery, such that theywould still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

It also is contemplated that various standard therapies, as well as surgical intervention, may be applied in combination with the described hyperproliferative cell therapy.

a. Chemotherapy

Cancer therapies also include a variety of combination therapies with both chemical and radiation based treatments. Combination chemotherapies include, for example, YM155, everolimus, withaferin A, parthenolide, vorinostat, scriptaid, olaparib, cisplatin, carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with the inventive therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

d. Genes

In yet another embodiment, the secondary treatment is a gene therapy in which a therapeutic polynucleotide is administered before, after, or at the same time as the invention. Delivery of a vector encoding either a full length or truncated therapeutic composition in conjuction with a second vector encoding one of the following gene products will have a combined anti-hyperproliferative effect on target tissues. Alternatively, a single vector encoding both genes may be used. A variety of proteins are encompassed within the invention, some of which include inducers of cellular proliferation, inhibitors of cellular proliferation, regulators of programmed cell death, and so forth.

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MP-theta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### VI. Pharmaceutical Preparations

Pharmaceutical compositions of the present invention comprise an effective amount of one or more therapeutic compositions dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one composition will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include the composition, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the composition may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In some embodiments of the present invention, the composition is formulated to be administered via an alimentary route Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft- shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally- administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, the composition may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VII. Kits of the Invention

Any of the compositions described herein may be comprised in a kit. In a non-limiting example, miRNAs, miRNA mimics, reagents for isolating miRNA, labeling miRNA, and/or evaluating an miRNA population may be included in a kit. The kit may further include reagents for synthesizing miRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits are also included as part of the invention. Kits for implementing methods of the invention described herein are specifically contemplated. In some embodiments, there are kits for using or preparing miR-34c. In these embodiments, the kit comprise, in suitable container means, one or more of the following: poly(A) polymerase; unmodified nucleotides (G, A, T, C, and/or U); 3) a modified nucleotide (labeled or unlabeled); poly(A) polymerase buffer; reaction buffer; solutions for preparing, isolating, enriching, and purifying miRNAs, and so forth. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism, or to a subset of such probes. The subset of probes on arrays of the invention may be or include those identified as relevant to a particular diagnostic, therapeutic, or prognostic application. For example, the array may contain one or more probes that is indicative or suggestive of a disease or condition or genetic predisposition to a disease or condition.

For any kit embodiment, including an array, there can be nucleic acid molecules that contain a sequence that is identical or complementary to all or part of any of SEQ ID NO:1 or 2. In certain embodiments, the nucleic acid is 80%, 85%, 90%, 95%, 97%, or 99% identical to SEQ ID NO:1 or 2. Any nucleic acid discussed herein may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the use or manipulation of miRNA.

### EXAMPLES

The following examples are offered by way of example and are not intended to limit the scope of the invention in any manner.

### Example 1

### High-Grade Serous Ovarian Cancer Arises from Fallopian Tube Mesenchyme

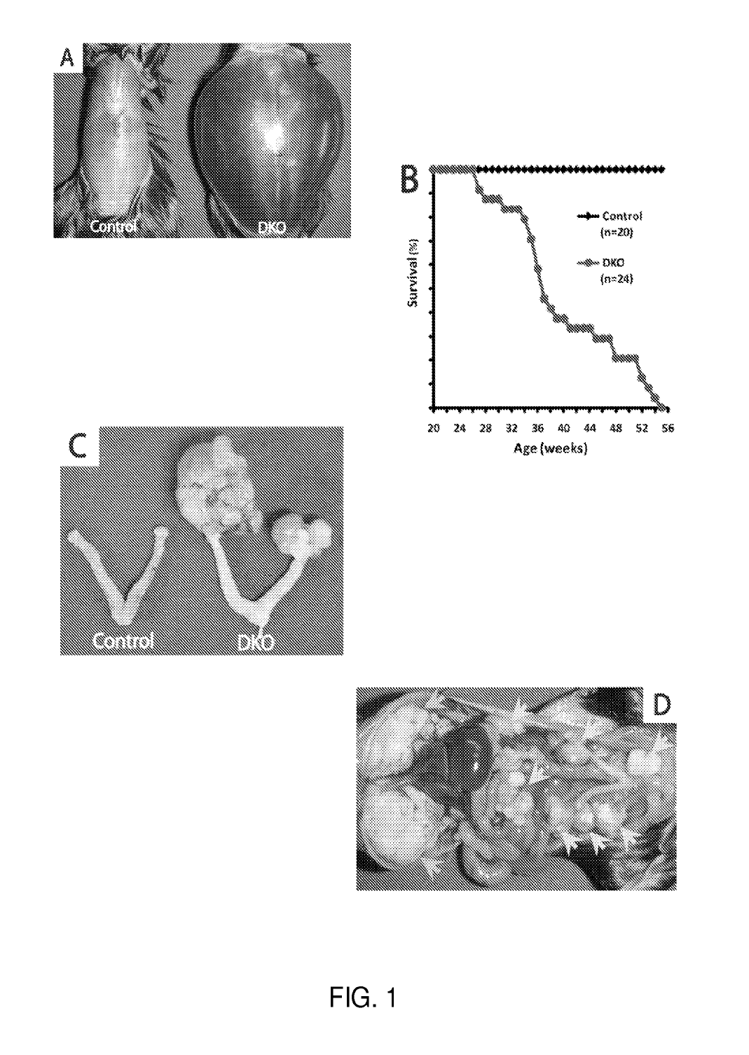

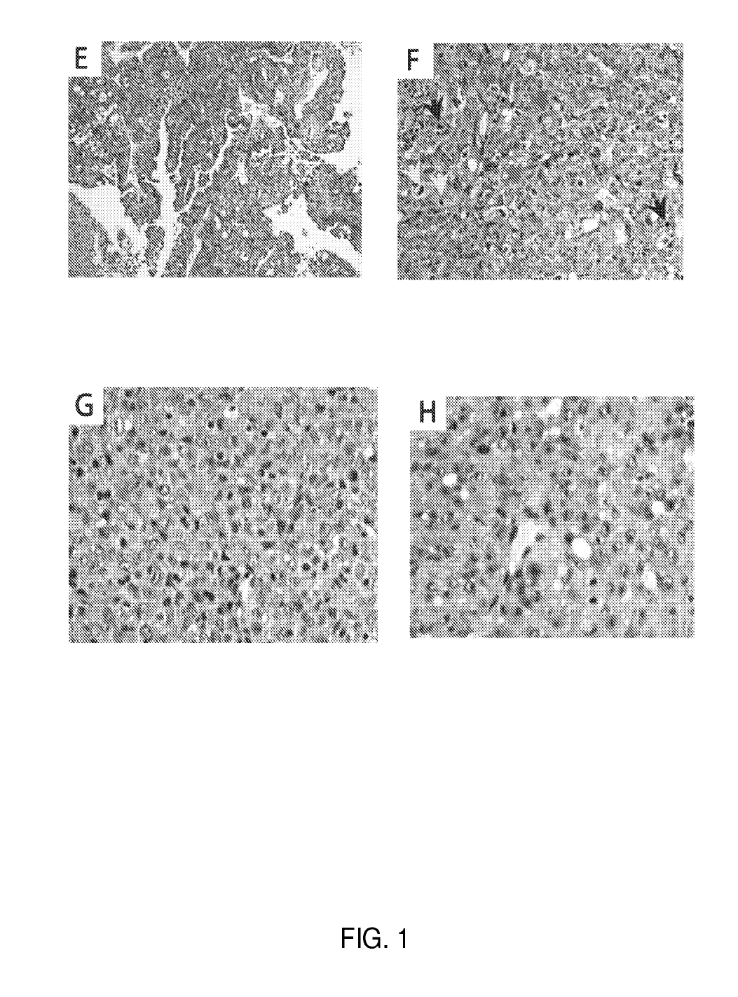

To define the in vivo relevance of the miRNA and PI3K/AKT/mTOR pathways in ovarian cancer, a mouse genetics strategy was used to conditionally delete both Dicer and the Pten tumor suppressor in the female reproductive tract using anti-Müllerian hormone receptor type 2 (Amhr2-Cre). These Dicer-Pten DKO (Dicer^{flox/flox} Pten^{flox/flox} Amhr2^{cre/+}) mice universally develop high-grade serous epithelial cancers (FIG. 1). The DKO mice grossly demonstrate ascites (FIG. 1A), and 100% of the DKO females succumb to death from the metastatic cancers between 26 and 55 weeks (FIG. 1B). Examination of the abdominal cavity shows that the cancers originate from the female reproductive tract in the vicinity of the ovary (FIG. 1C). These malignant tumors then aggressively metastasize throughout the abdominal cavity, with prominent cancers lesions on the diaphragm, mesentery, and peritoneal membrane (FIG. 1D). Histologically, the cancers are characterized by complex papillae and glands forming slit-like spaces as well as solid sheets of tumor cells (FIG. 1E) with pleomorphic nuclei, prominent nucleoli, and high mitotic activity (FIG. 1F, G)—cardinal features of high-grade serous ovarian cancer in humans. These high-grade serous carcinomas are reproducible in vivo. When cells isolated from primary tumors, ascites, or metastatic tumors were injected intraperitoneally into immunocompromised (NOD SCID) or immunocompetent mice, the injected mice (11 out of 11 mice for NOD SCID; 9 out of 13 for immunocompetent mice) developed histologically identical high-grade serous carcinomas (FIG. 1H).

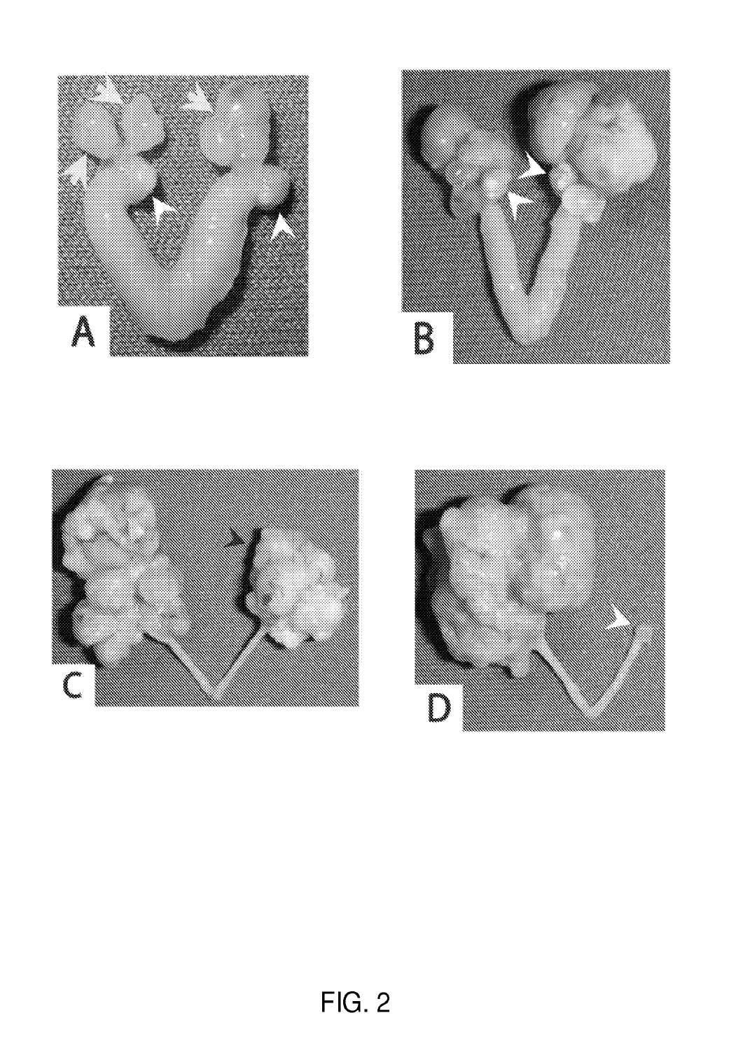

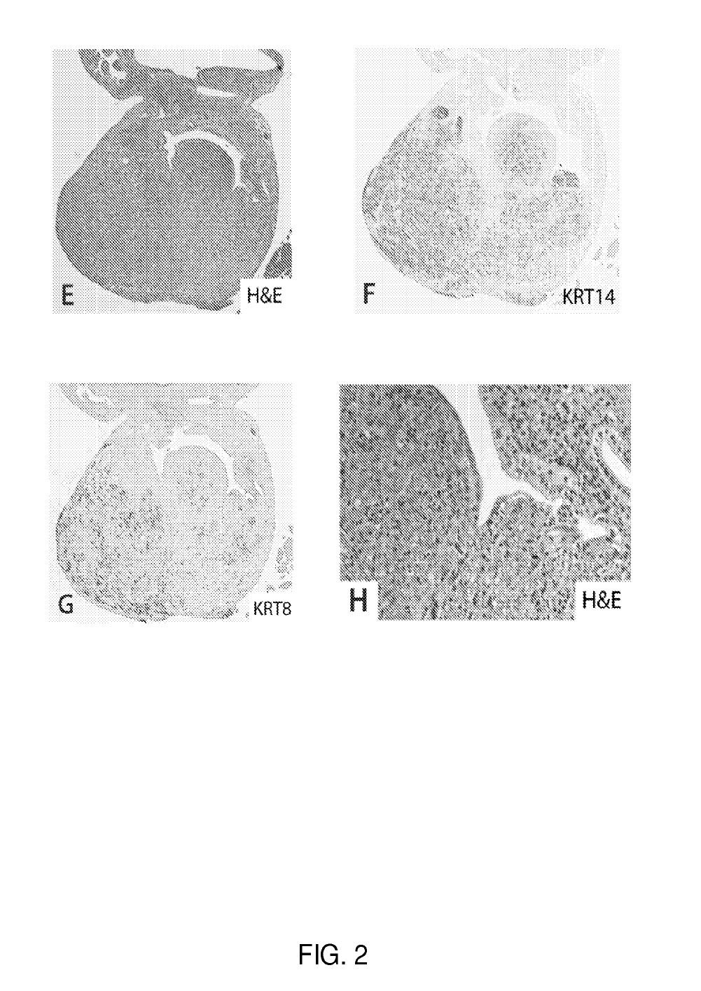

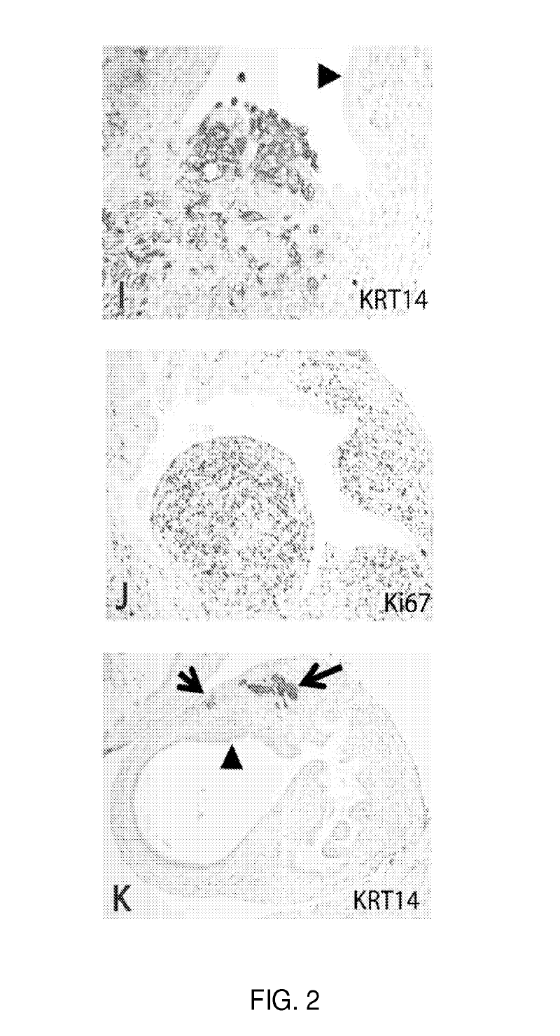

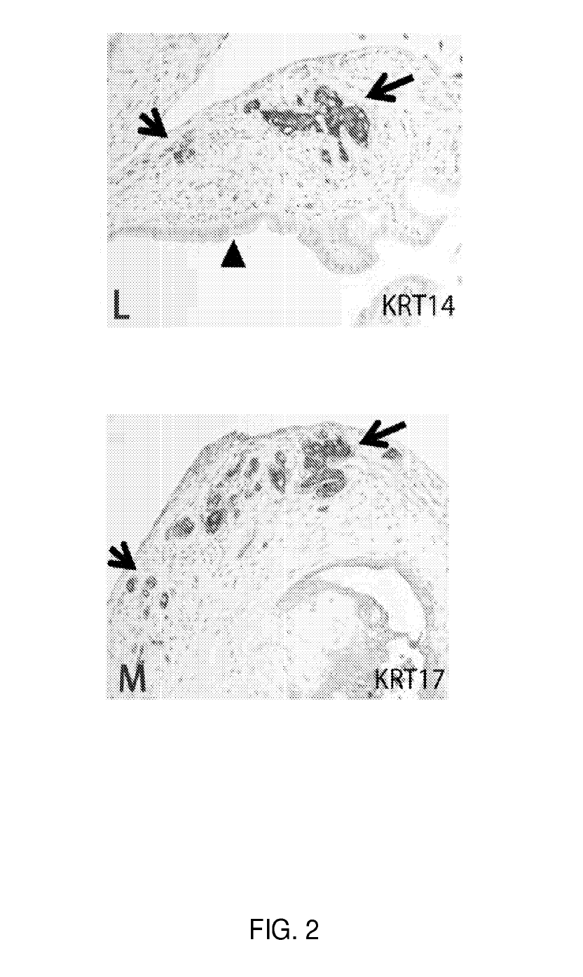

To determine the cell origin of these serous carcinomas, the Dicer-Pten DKO mice were analyzed at earlier time points before the development of ascites and metastasis. The high-grade serous carcinomas in the DKO mice arise from the fallopian tube (FIG. 2A, B), and then spread to the ovary and metastasize to the peritoneum (FIG. 1C, D). In mice with early fallopian tube tumors, the ovaries are grossly distinguishable from the fallopian tube serous tumors, histologically remain intact, and show no signs of tumor (FIG. 2A, B and Supplemental figure). These fallopian tube tumors are unique to the DKO mice since Amhr2-Cre deletion of Dicer alone leads to the formation of diverticuli in the fallopian tube and no tumors (Nagaraja et al., 2008), while disabling Pten does not cause any tumor phenotype in the ovary or fallopian tube (Fan et al., 2009).

To further confirm the fallopian tube origin of the cancers, the ovary or fallopian tube was unilaterally removed from the DKO mice. Even after one of the ovaries is surgically removed from a DKO mouse at postnatal 6-11 weeks, the fallopian tube alone can still form tumors in four out of five mice) (FIG. 2C). However, cancers fail to form upon unilateral removal of the fallopian tube (four out of four mice), despite the presence of the ovary (FIG. 2D). These results further confirm that the serous cancers arise from the fallopian tube.

Histologic analysis of the fallopian tubes from the DKO mice at earlier ages shows that the abnormal proliferation begins in the stromal compartment of the fallopian tube (FIG. 2F-M). Tumor lesions similar to those in FIG. 2A fill the stromal compartment of the fallopian tube and compress the lumen (FIG. 2E). The appearance of these cancers in the stromal compartment is consistent with the activity of Cre in the stroma of the fallopian tube and uterus where Amhr2 is expressed (Arango et al., 2008).

To identify putative markers for these cancers, RNA was isolated from the fallopian tube cancers of independent DKO mice and normal fallopian tubes of control mice and subjected to mRNA expression analysis using the Illumina platform (MouseWG-6 v2 Expression BeadChip). Several epithelial markers were upregulated in the serous cancers compared with the normal fallopian tube including cytokeratin 14 (KRT14; 198.3-fold increased), cytokeratin 8 (KRT8), E-cadherin (CDH1), and cytokeratin 17 (KRT17; 14.3-fold increased) (Table 1).

TABLE 1
Genes encoding secreted and/or transmembrane proteins
upregulated in mouse fallopian tube carcinomas and human
serous carcinomas versus respective fallopian tubes (FT).
Mean expression levels of independent samples of mouse
fallopian tubes (n = 3) and fallopian tube serous cancers
(n = 3) are shown. Fold changes in gene expression are
compared between mouse fallopian tube cancer and human
serous ovarian cancer with their respective fallopian tubes
as controls.

| | | Expression Level | | Fold Change | |
|---|---|---|---|---|---|
| | | | Mouse FT | Mouse | Human |
| Symbol | Gene Name | Mouse FT | cancer | cancer: FT | cancer: FT |
| Spp1 | Secreted | 169 | 15,370 | 102.7 | 39.5 |
| | phospho- | | | | |
| | protein 1 | | | | |
| Cxcl9 | Chemokine | 56 | 2,268 | 33.8 | 4.1 |
| | (C—X—C | | | | |
| | motif) | | | | |
| | ligand 9 | | | | |
| Cxcl10 | Chemokine | 56 | 1,951 | 25.8 | 1.9 |
| | (C—X—C | | | | |
| | motif) | | | | |
| | ligand 10 | | | | |
| Cd72 | CD72 | 216 | 2,955 | 13.7 | 1.9 |
| | antigen | | | | |
| Slc15a3 | Solute | 84 | 1,168 | 13.0 | 4.6 |
| | carrier | | | | |
| | family 15, | | | | |
| | member 3 | | | | |
| Cd84 | CD84 | 101 | 1,275 | 12.5 | 1.7 |
| | antigen | | | | |
| C1qb | Complement | 1368 | 13,752 | 10.0 | 6.5 |
| | component | | | | |
| | 1qB | | | | |
| Plau | Plasminogen | 75 | 747 | 9.8 | 4.8 |
| | activator, | | | | |
| | urokinase | | | | |
| Ly86 | Lymphocyte | 566 | 4,479 | 7.8 | 3.1 |
| | antigen 86 | | | | |
| Muc16 | Mucin 16 | 435 | 3,508 | 7.6 | 26.1 |
| | (CA125) | | | | |
| Folr1 | Folate | 204 | 1,590 | 7.3 | 77.6 |
| | receptor 1 | | | | |
| Slc11a1 | Solute | 154 | 1,122 | 7.2 | 2.1 |
| | carrier | | | | |
| | family 11, | | | | |
| | member 1 | | | | |
| Slc12a8 | Solute | 71 | 531 | 7.2 | 4.8 |
| | carrier | | | | |
| | family 12, | | | | |
| | member 8 | | | | |
| Cd40 | CD40 | 100 | 684 | 6.8 | 2.5 |
| Igsf9 | Immuno- | 602 | 3311 | 5.4 | 9.1 |
| | globulin | | | | |
| | superfamily, | | | | |
| | member 9 | | | | |
| II10ra | Interleukin | 85 | 450 | 5.3 | 2.0 |
| | 10 receptor, | | | | |
| | alpha | | | | |
| Tnfrsf12a | Tumor | 265 | 1351 | 5.3 | 2.7 |
| | necrosis | | | | |
| | factor | | | | |
| | receptor, | | | | |
| | member 12a | | | | |
| Apoe | Apolipo- | 2573 | 13,702 | 5.1 | 1.7 |
| | protein E | | | | |
| Tlr7 | Toll-like | 81 | 429 | 5.1 | 2.2 |
| | receptor 7 | | | | |
| Tmem48 | Trans- | 152 | 705 | 4.7 | 5.2 |
| | membrane | | | | |
| | protein 48 | | | | |
| II1r2 | Interleukin 1 | 44 | 195 | 4.4 | 1.8 |
| | receptor, | | | | |
| | type II | | | | |
| Lair1 | Leukocyte- | 76 | 319 | 4.1 | 20.1 |
| | associated | | | | |
| | Ig-like | | | | |
| | receptor 1 | | | | |
| Ly6e | Lymphocyte | 1580 | 6,370 | 4.1 | 33.3 |
| | antigen 6 | | | | |
| | complex, | | | | |
| | locus E | | | | |
| Adam17 | A disintegrin | 488 | 1,975 | 4.0 | 2.0 |
| | and | | | | |
| | metallo- | | | | |
| | peptidase | | | | |
| | domain 17 | | | | |
| Ptn | Pleiotrophin | 1202 | 4,648 | 3.8 | 1.9 |
| Cd83 | CD83 | 173 | 603 | 3.6 | 5.0 |
| | antigen | | | | |
| Ccl8 | Chemokine | 1106 | 3,961 | 3.6 | 12.0 |
| | (C-C motif) | | | | |
| | ligand 8 | | | | |
| Tmc6 | Trans- | 231 | 730 | 3.2 | 5.2 |
| | membrane | | | | |
| | channel-like | | | | |
| | gene | | | | |
| | family 6 | | | | |
| Tmem49 | Trans- | 1169 | 3,369 | 2.9 | 8.3 |
| | membrane | | | | |
| | protein 49 | | | | |
| Esm1 | Endothelial | 106 | 288 | 2.7 | 4.6 |
| | cell-specific | | | | |
| | molecule 1 | | | | |
| Amhr2 | Anti- | 184 | 475 | 2.6 | 5.4 |
| | Mullerian | | | | |
| | hormone | | | | |
| | type 2 | | | | |
| | receptor | | | | |
| Mdk | Midkine | 1551 | 4,092 | 2.6 | 1.8 |
| Tmem173 | Trans- | 89 | 233 | 2.6 | 3.7 |
| | membrane | | | | |
| | protein 173 | | | | |
| Tnfrsf21 | Tumor | 260 | 642 | 2.6 | 14.6 |
| | necrosis | | | | |
| | factor | | | | |
| | receptor, | | | | |
| | member 21 | | | | |
| Cfb | Complement | 184 | 439 | 2.3 | 9.1 |
| | factor B | | | | |
| Scamp5 | Secretory | 523 | 1,114 | 2.1 | 2.6 |
| | carrier | | | | |
| | membrane | | | | |
| | protein 5 | | | | |

Immunohistochemical analysis using these epithelial markers demonstrate that most of the cancer cells in the stromal compartment are expressing these proteins (FIG. 2 F, G, I, K, L, M). KRT14, a highly specific marker for the cancers, is essentially negative in the normal luminal epithelium of the fallopian tube, but is expressed in some cancer cells that are invading the lumen (FIG. 2 F, I). Using an antibody to Ki67, these tumor lesions were observed to be highly proliferative (FIG. 2J).

To further determine the origin of these cancers, the inventors used the anti-KRT14 and anti-KRT17 antibodies to study regions of the fallopian tube that did not contain grossly obvious cancer. The specificity of these antibodies allowed us to uncover focal KRT14+, KRT17+ serous cancer lesions in the stromal compartment (FIG. 2K-M). These results further indicate that the epithelial serous adenocarcinomas originate from mesenchymal cells in the stroma of the fallopian tube.

Mesenchymal-to-epithelial transition (MET) is known to occur during development, notably in kidney formation (Rothebpieler et al., 1993). In Huang et al. (2011), MET also plays a physiological role in an adult tissue. During uterine regeneration that occurs after delivery in mice, some Amhr2-expressing mesenchymal-lineage stromal cells convert to epithelial cells, contributing to the epithelial layers of the uterine lumen and glands (Huang et al., 2011). Like uterus, mouse fallopian tube has stromal cells of a mesenchymal origin expressing Amhr2 (Arango et al., 2008). Thus, the results indicate a novel paradigm in which high-grade serous adenocarcinomas initiate through a unique mesenchymal-to-epithelial transition (MET).

By identifying the fallopian tube as the site of origin of the serous carcinomas allowed us to examine early genetic changes in the development of serous ovarian cancer. The microarray gene-expression analysis reveals numerous highly expressed genes that could potentially be important for initiating early events of ovarian cancer. Some of these upregulated genes are secreted or transmembrane proteins (Table 1).

The list shows several intriguing genes including secreted phosphoprotein 1 (Sppl), CA125 (Muc16), folate receptor 1 (Folr1), and chemokines such as Cxc19, Cxc110, and Ccl8. Along with CA125, SPP1 has been suggested as a putative serum biomarker that can detect early ovarian cancer with a high sensitivity and specificity (Meinhold-Heerlein et al., 2007). In addition, mRNAs encoding the chemokine CCL8 have been detected in ascites cells of >85% of women with epithelial ovarian cancer (Milliken et al., 2002). FOLR1 is highly expressed in 90% of women with epithelial ovarian cancer, and the overexpression of this receptor is associated with high grade and advanced stage (Kalli et al., 2008). These proteins, therefore, are likely critical markers for early detection and screening of ovarian cancer.

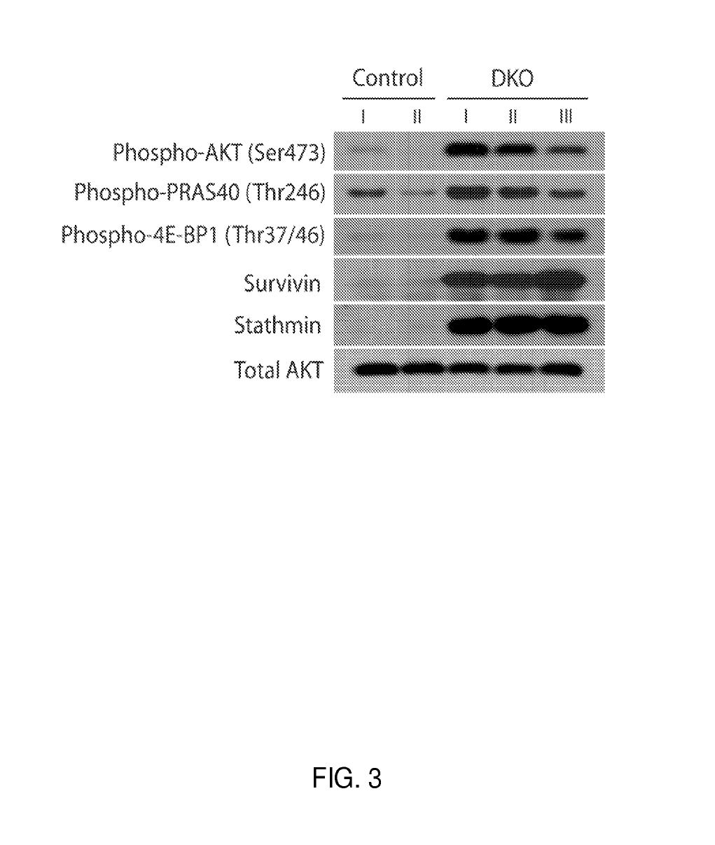

Using next generation sequencing of human serous carcinomas, it was shown that individual miRNAs, such as miR-31 and miR-100, are expressed at low levels in human serous ovarian carcinomas, suggesting that they are potential tumor suppressors (Creighton et al., 2010). miR-31 impinges on the E2F pathway (Creighton et al., 2010), while miR-100 can suppress the PI3K pathway (Nagaraja et al., 2010). In these DKO mice, Pten absence breaks the tight regulatory loop comprising PTEN (phosphatase) and PI3K (kinase) (Bunney and Katan, 2010), resulting in aberrantly activated AKT and increased phosphorylation of AKT downstream proteins (FIG. 3). The serous adenocarcinomas also show a dramatic rise in the expression of the Birc5 (survivin) mRNA (16.4-fold up regulation in the mouse tumors versus normal fallopian tubes; 5.4-fold up regulation in the human serous cancers versus normal fallopian tubes) as well as the survivin protein (highly expressed in mouse tumors but almost undetectable in normal fallopian tubes) (FIG. 3). Well known as an apoptosis inhibitor, survivin was uncovered as a cancer gene since its discovery in 1997. Survivin has been identified to contribute to nearly every aspect of cancer, from onset to outcome (Altieri, 2008). Although there is evidence showing that enhanced surviving expression is regulated by the aberrantly activated PI3K pathway (Martinelli et al., 2006), other studies have indicated that p53 directly suppresses survivin expression (Hoffman et al., 2002; Mirza et al., 2002). Given that p53 is mutated in most serous adenocarcinomas (Cho and Shih, 2009), highly expressed survivin may be implicated in the onset of human serous carcinomas similar to the animal model. STMN1 (stathmin), another downstream target of PI3K involving in cytoskeletal reorganization (Salvesen et al., 2009), was also highly expressed in early fallopian tube tumors at the mRNA and protein levels (FIG. 3).

The findings are the first to show the in vivo progression of high-grade serous epithelial cancer, which begins from lesions in the fallopian tube and then spreads to the ovaries, ultimately leading to widespread peritoneal metastasis ending in death. Besides identifying fallopian tube as the origin of the cancer, the inventors have also uncovered MET as a novel mechanism of high-grade serous cancer initiation. The studies therefore present a new paradigm to understand the origin and progression of epithelial ovarian cancer. This information is vital for identifying biomarkers for early detection and screening. Because high-grade ovarian cancers are detected at advanced stages with high mortality, the mouse model helps discover new drug targets and pathways for treating advanced ovarian cancers. This model is critical for translational inroads in a “war on ovarian cancer.”

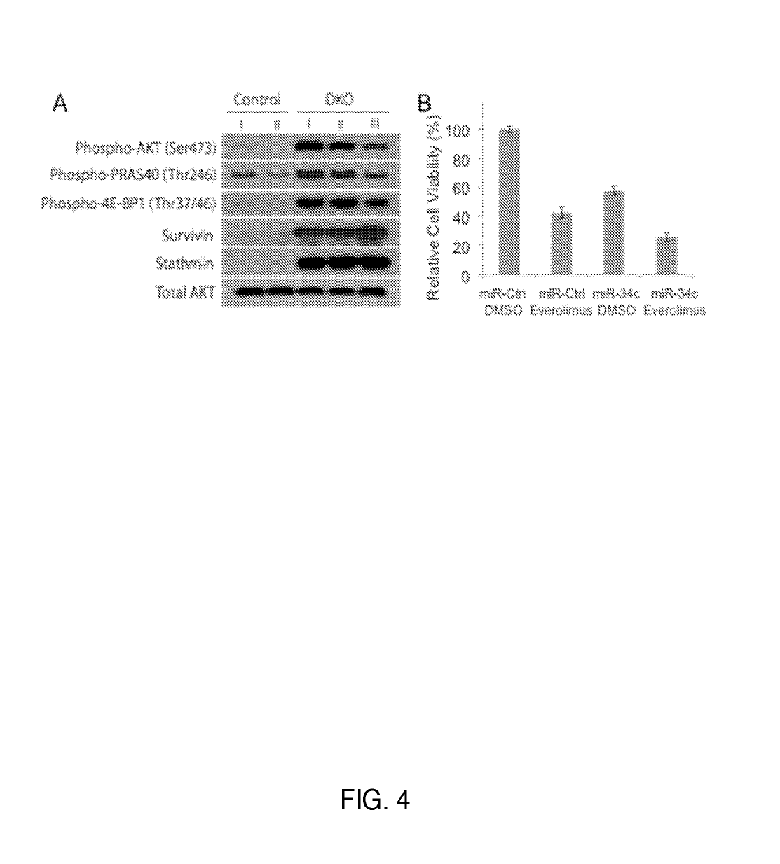

Multiple studies have demonstrated activation of the three miR-34 family members by p53, and mir-34 overexpression induces cell cycle arrest or apoptosis depending on the cellular context. Reduced miR-34 expression has been reported in ovarian cancer, neuroblastoma, pancreatic cancer, and non-small cell lung cancer. Furthemore, miR-34b/c expression in colorectal cancer is epigenetically regulated, methylation of the miR-34b/c promoter is associated with poor prognosis in non-small cell lung cancer, and methylation at the mir-34a and mir-34b*/c loci was observed in 27% (8 of 30) and 47% (14 of 30) of ovarian cancer samples, respectively. Using miRNA microarrays, miR34c levels were also found to be the sole independent predictor of serous ovarian cancer recurrence-free survival. Using next generation sequencing, miR-34c was the fifth most abundant miRNA in fallopian tube and the most common non-let-7 family miRNA, comprising 4.2% of all fallopian tube miRNAs. In contrast, seven out of eight human serous carcinomas showed a dramatic depletion of miR-34c to 0.12% (range: 0.024-0.40%) of the total miRNAs in each tumor. The PI3K pathway is highly activated in the primary fallopian tube carcinomas compared to fallopian tube (FIG. 4A).

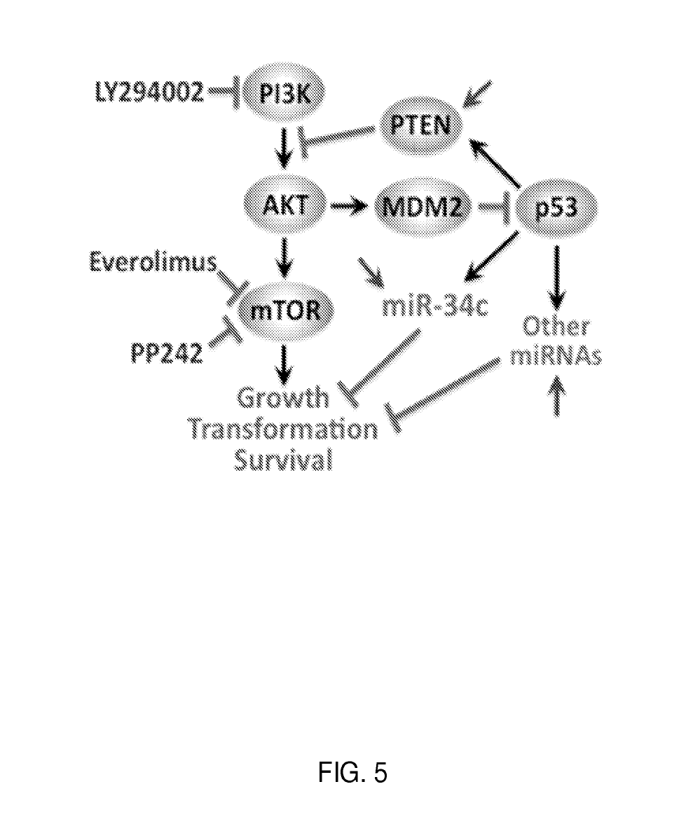

To functionally evaluate the roles of miR34c and the mTOR inhibitor everolimus in serous cancers, the inventors first generated 32 cell lines from primary cancers (5 lines), metastatic lesions (11 lines), and ascites fluid (16 lines) of independent Dicer/Pten DKO mice. The effects were analyzed of everolimus and miR-34c mimic alone and in combination on a confirmed Dicer/Pten double null cell line derived from a primary mouse serous cancer. The cells were exquisitely sensitive to everolimus and/or miR-34c which had significant anti-proliferative (secondary to G1 arrest) and pro-apoptotic effects (FIG. 4B). Based on the studies, there are the following considerations: 1) MiR-34c and additional miRNAs function to suppress the development of high grade serous carcinomas and the mesenchymal-to-epithelial transition, and/or 2) Simultaneous pharmacological targeting of mTOR and pathways downstream of miR-34c and other miRNAs leads to therapies to eradicate high-grade serous carcinomas. An exemplary model is shown in FIG. 5.

### Example 2

### Exemplary Materials and Methods

Generation of Dicer-Pten Conditional Double-Knockout (Dicer-Pten DKO) Mice

Dicer and Pten were conditionally disabled in mice by Amhr2-Cre. To generate the Dicer-Pten DKO mice, Dicer^{flox/flox} or Dicer^{flox/-} mice and Pten^{flox/flox} or Pten^{flox/-} mice were mated with Amhr2^{cre/+} mice. A series of breedings generated Dicer-Pten DKO mice of four genotypes: (1) Dicer^{flox/flox} Pten^{flox/flox} Amhr2^{cre/+}; (2) Dicer^{flox/-} Pten^{flox/flox} Amhr2^{cre/+}; (3) Dicer^{flox/flox} Pten^{flox/-} Amhr2^{cre/+}; and (4) Dicer Dicer^{flox/-} Pten^{flox/-} Amhr2^{cre/+}. These genotype differences did not appear to affect the onset of tumor formation, severity of metastasis, and mouse survival. Respective genotypes not carrying Amhr2^{cre/+} (for example, Dicer^{flox/flox} Pten^{flox/flox) were used as control mice.}

In Vivo Reproduction of Dicer-Pten-DKO High-Grade Serous Ovarian Carcinoma

Tumor-forming ability of the high-grade serous cancers in Dicer-Pten DKO mice was tested in severe combined immunodeficiency (NOD SCID) or immunocompetent (C57BL/129Sv) mice. Cells separated from serous ovarian tumors, metastatic tumors, or ascites were injected intraperitoneally into NOD SCID or C57BL/129Sv mice. Tumors that were developed from these injections were histologically examined after hematoxylin and eosin (H&E) staining.

Microarray Analysis

Early gene-expression changes in the fallopian tube tumors were investigated by microarrays. Total RNA was isolated, using Trizol® (Invitrogen), from early fallopian tube tumors (FIG. 2A) of Pten-Dicer DKO mice and from normal fallopian tubes of control mice. After the RNA was cleaned with Dnase I, the total RNA was converted first to cDNA and then to biotin-labeled cRNA, which were hybridized with oligonucleotides, representing more than 47,000 transcripts, on a gene array (MouseWG-6 v2 Expression BeadChip, Illumina). Using the gene expression levels in normal fallopian tube as controls, genes whose expression was differentially expressed in mouse carcinomas with P<0.01, FDR<0.05 in early fallopian tube tumors were identified (see below). Expression data were quantile normalized.

In addition, expression profiles of human fimbria (n=2) were compared with previously published profiles of human ovarian serous cancers (n=8) (Creighton et al., 2010) (the fimbria and cancer sample profiles having been generated within the same time frame). Array datasets have been deposited into the Gene Expression Omnibus (GEO Accession numbers pending).

Differentially expressed genes were identified using t-test and fold change on log-transformed data (p-values were two-sided). The method of Storey et al. (2003) was used to estimate the false discovery rate (FDR) from multiple hypothesis testing; of the ˜47,000 MouseWG-6 v2 gene probe in the entire dataset, 10821 were nominally significant with nominal P<0.01 (no fold criteria), which yielded an FDR of 4%.

Immunohistochemistry

Mouse tumor tissues were fixed in 10% formalin at room temperature (RT) for 24 h, embedded in paraffin, sectioned at 4 μm, and mounted on slides (Superfrost Plus®, Fisher Scientific). After deparaffinized in xylene (three times for 5 min each), the slides were then gradually hydrated in decreasing concentrations of ethanol, and finally in tap water: (1) 100% ethanol (three times for 5 min each); (2) 95% ethanol (once for 5 min); (3) 70% ethanol (once for 5 min); (4) 50% ethanol (once for 5 min); (5) tap water (once for 5 min)

For antigen retrieval, these slides were microwaved in a citrate buffer (0.0082M sodium citrate, 0.0018M citric acid, pH6) for 10 min twice, followed by cooling at RT for 1 h. Then, potential endogenous peroxidase activity in tumor tissues was blocked by incubating the slides in 3% H₂O₂ (in methanol) at RT for 10 min in the dark. Endogenous activity of biotin and avidin also was blocked by incubating the slides sequentially in avidin and biotin at RT for 10 min each (Vectastain® Avidin Biotin blocking kit). To further reduce non-specific reaction, the slides were incubated in 5% normal goat serum in blocking buffer (3% bovine serum albumin and 0.1% triton X-100 in phosphate buffered saline) at RT for 1 h. The slides were then incubated with an antibodies to KRT8 (Abcam; rabbit; 1:50 in blocking buffer), KRT14 (Covance; rabbit; 1:1,000), Ki67 (BD Pharmingen; mouse; 1:1,000), or E-cadherin (CDH1) (Cell Signaling Technology; rabbit; 1:100) at 4° C. overnight.

To detect the primary antibody, the slides were incubated with biotinylated goat anti-rabbit or -mouse IgG (Vector Laboratories, 1:200 in blocking buffer) at RT for 1 h. This biotinylated secondary antibody was then detected by incubating the slides with streptavidin-linked horseradish peroxidase (HRP) (Vectastain® ABC kit) at RT for 30 min The slides were then stained with DAB substrate (Vectastain® DAB kit) to detect the antibody-bound HRP. After dehydrated first by processing backward the steps of gradual ethanol hydration and then three times in xylene (for 5 min each), the slides were covered with a coverslip in mounting medium (Permount™, Fisher Scientific) and examined under a light microscope.

Western Blot

Activation of PI3K signaling was examined in early Dicer-Pten-DKO fallopian tube tumors by western blot analysis. Protein extracts were prepared from early fallopian tube tumors and normal fallopian tubes in RIPA buffer (50 mM Tris-HCl pH7.4, 150 mM NaCl, 1% NP40, 0.5% sodium deoxycholate, 0.1% SDS, 5 mM EDTA, 1 mM EGTA). Ten ug each of tumor or normal protein extracts were electrophoresesed on a 4-12% gradient NuPage gel, and then transferred to a PVDF membrane at 20V overnight at RT.

After blocked in 5% milk, this membrane was incubated at RT for 1 h with a primary antibody for phospho-AKT (Ser473) (Cell Signaling Technology; rabbit; 1:2,000), phospho-PRAS40 (Thr246), phospho-4E-BP1 (Thr37/46) (Cell Signaling Technology; rabbit; 1:1,000), survivin (Cell Signaling Technology; rabbit; 1:1,000), stathmin (Cell Signaling Technology; rabbit; 1:1,000), or total AKT (Cell Signaling Technology; rabbit; 1:2,000). Primary antibody incubation was followed by incubating the membrane with an anti-rabbit IgG linked to horseradish peroxidase (HRP) (Jackson ImmunoResearch; Goat; 1:3,750) at RT for 1 h. After reacted with a substrate (SuperSignal® West Pico Chemiluminescent Substrate, Thermo Scientific) for 5 min to detect antibody-bound HRP, the membrane was exposed to an X-ray film.

### Example 3

### MIR-34C as a Tumor Suppressor MIRNA

Despite advances in surgery and chemotherapy, women who are diagnosed with high-grade serous ovarian cancer, the histologic cancer type that causes 70% of ovarian cancer deaths, have a poor five-year survival rate of 31%. Recent studies suggest that low-grade serous ovarian cancers originate from the surface epithelium of the ovary, whereas high-grade “ovarian” serous cancers (90% of serous cancers) originate from the fallopian tube and spread to the ovary and peritoneum. However, because the majority of high-grade serous carcinomas are detected at late stages and ultimately kill 90% of these women, in vivo models are needed to define the origin and progression of serous carcinomas, identify biomarkers for early detection, and test novel therapeutic strategies for eradicating the cancer.

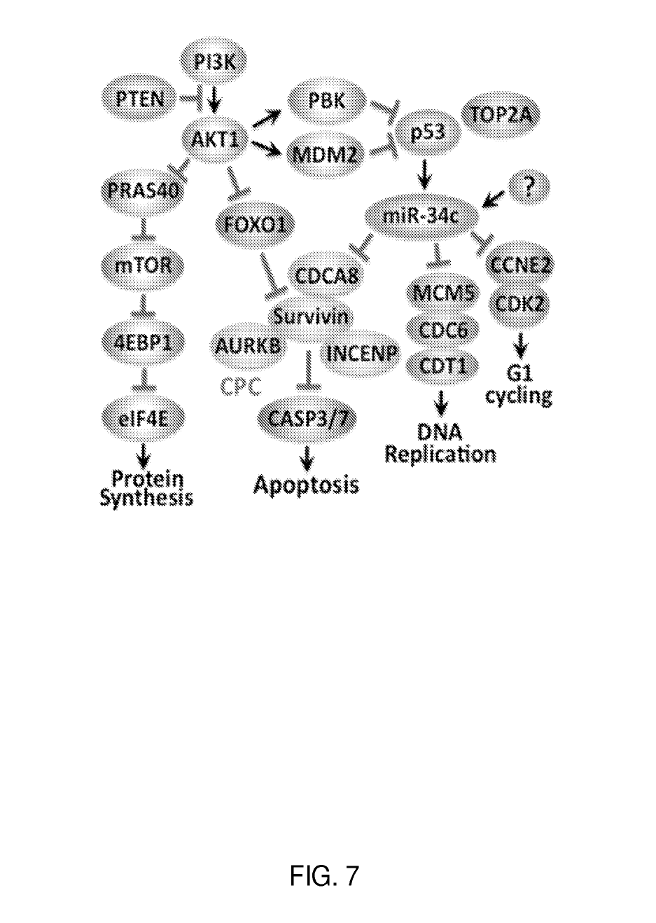

MicroRNAs (miRNAs) are ˜22 nucleotide non-coding RNAs that bind to the 3′ untranslated regions of mRNAs to repress translation and induce degradation of multiple mRNAs (Du et al., 2004; Edson et al., 2009). High levels of the miRNA biosynthesis enzymes, DICER and DROSHA, correlate with increased survival for ovarian cancer patients, mutations that activate the PI3K/RAS pathway are observed in 45% of ovarian cancers, and 70% of serous cancers lack expression of PTEN, the negative regulator of the PI3K pathway. To define the in vivo relevance of miRNA and PI3K pathways in ovarian cancer, DICER and PTEN were conditionally deleted in the female reproductive tract. These DICER/PTEN DKO mice develop high-grade serous epithelial cancers of the fallopian tube that engulf the ovary, metastasize to the peritoneum, and cause ascites and 100% death by 13 months. If one removes the fallopian tubes from these DKO mice, the mice fail to develop cancer, but ovariectomized mice continue to develop high-grade metastatic serous carcinomas, confirming the tubal origin of these cancers. Analysis of the gene expression profiles of the fallopian tube cancers uncovered gene products that are biomarkers for high-grade serous carcinomas [e.g., CA125, secreted phosphoprotein 1 (SPP1), folate receptor 1 (FOLR1) and several cytokines] and as therapeutic targets (e.g., components of the chromosomal passenger complex (CPC) including survivin, and the minichromosome maintenance complex such as MCM5), in particular embodiments of the invention. Mouse and human high-grade serous carcinomas express survivin mRNA and protein at high levels and apoptose at nanomolar concentrations of the novel survivin inhibitor, YM155. MiR-34c is highly expressed in the fallopian tube (4.2% of all miRNAs) and suppressed 83-fold in high-grade serous ovarian cancers. Because miR-34 family members are downstream targets of p53 and low levels of miR-34c in serous ovarian cancer are associated with decreased patient survival, in embodiments of the invention suppression of miR-34c (secondary to p53 mutation or deletion or epigenetic changes in the MIR34B/C locus) is a driving force in serous carcinomas. When one transfects miR-34c mimics into mouse and human serous carcinoma cell lines, miR-34c reduced cell viability and regulated multiple cell cycle and apoptotic pathways, allowing for the development of a model for serous cancer (FIG. 7).

In some embodiments of the invention, the molecular pathogenesis of high-grade serous carcinomas during metastasis is characterized. In some embodiments of the invention, the roles of miR-34c in serous cancer initiation and progression are defined. In some embodiments of the invention, the relevance of novel biomarkers for high-grade serous carcinomas is characterized. In some embodiments of the invention, unique therapeutic approaches to treat serous carcinomas are uncovered.

The studies provided herein are the first to demonstrate the in vivo progression of high-grade serous epithelial cancer from lesions in the fallopian tube to metastatic cancer. These studies will jumpstart the knowledge in the art of the molecular pathways (e.g., miR-34c-regulated) pathways involved in the initiation and metastatic progression of high-grade serous carcinoma, define early biomarkers, and identify small molecules that can synergize to eradicate serous carcinomas.

### Example 4

### Exemplary Research Strategy

### Significance

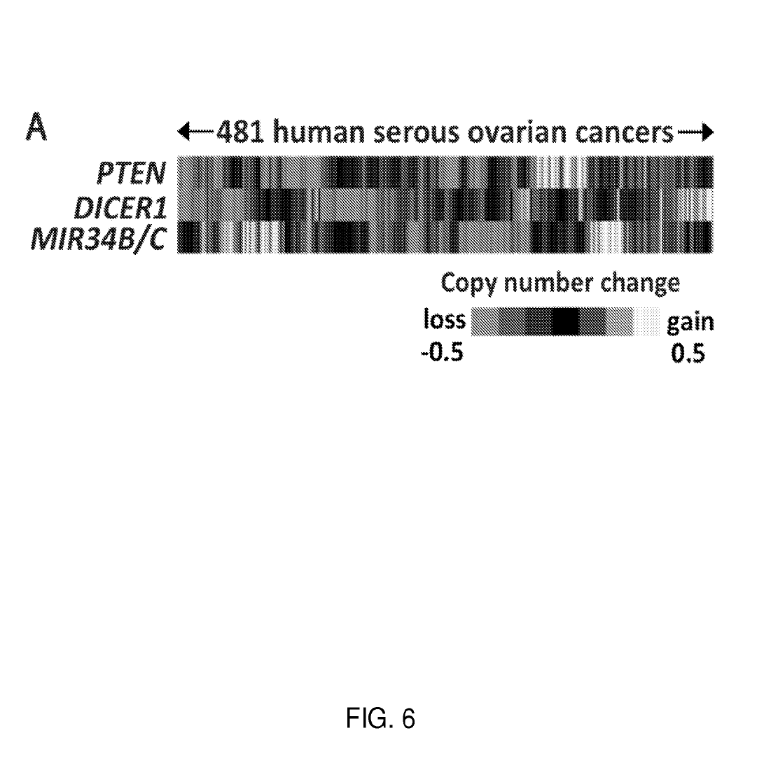

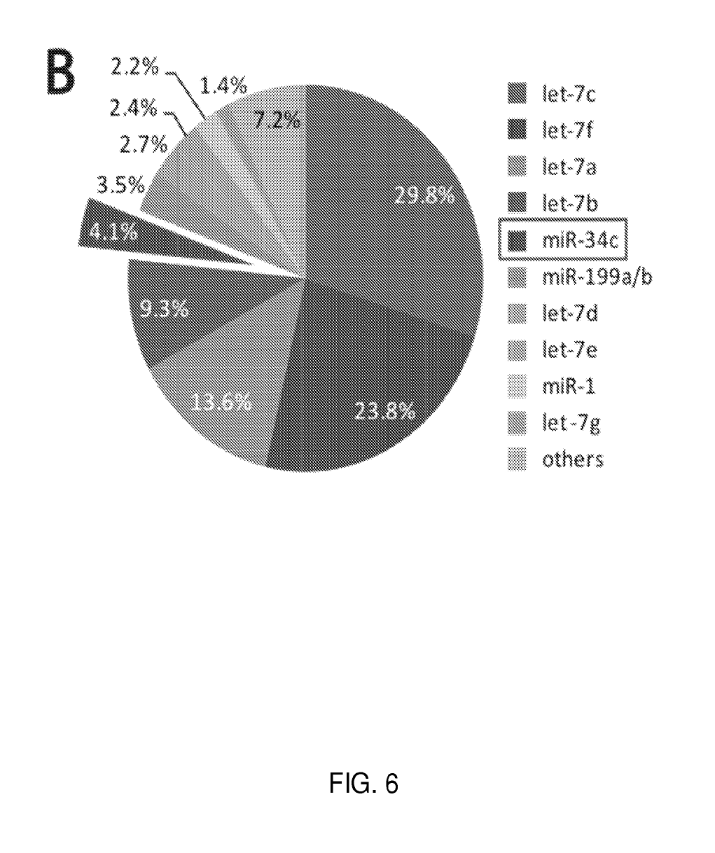

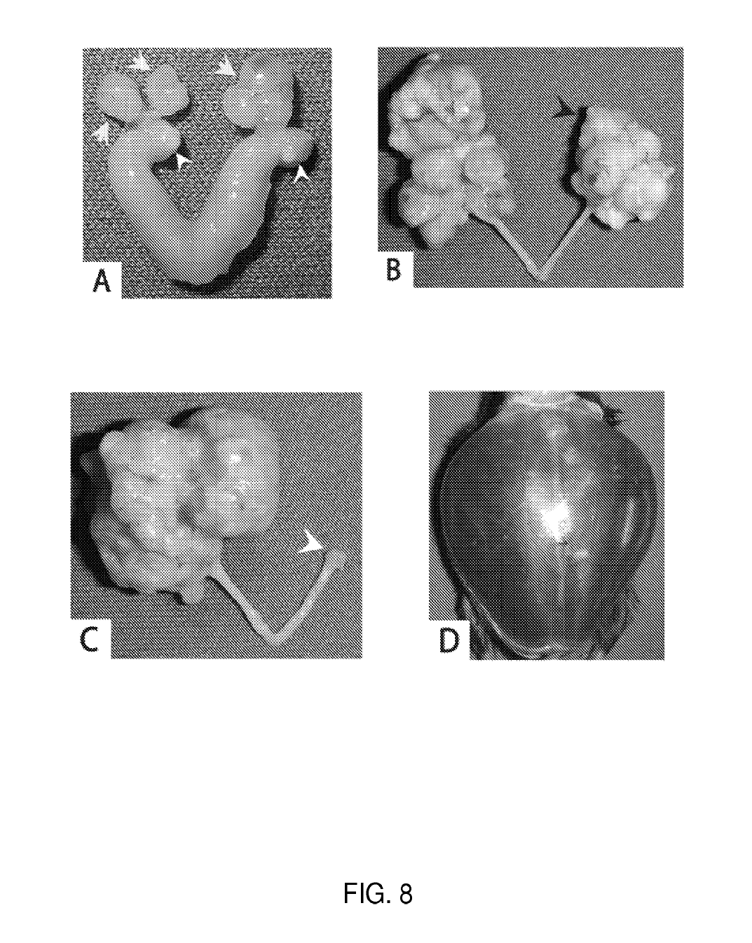

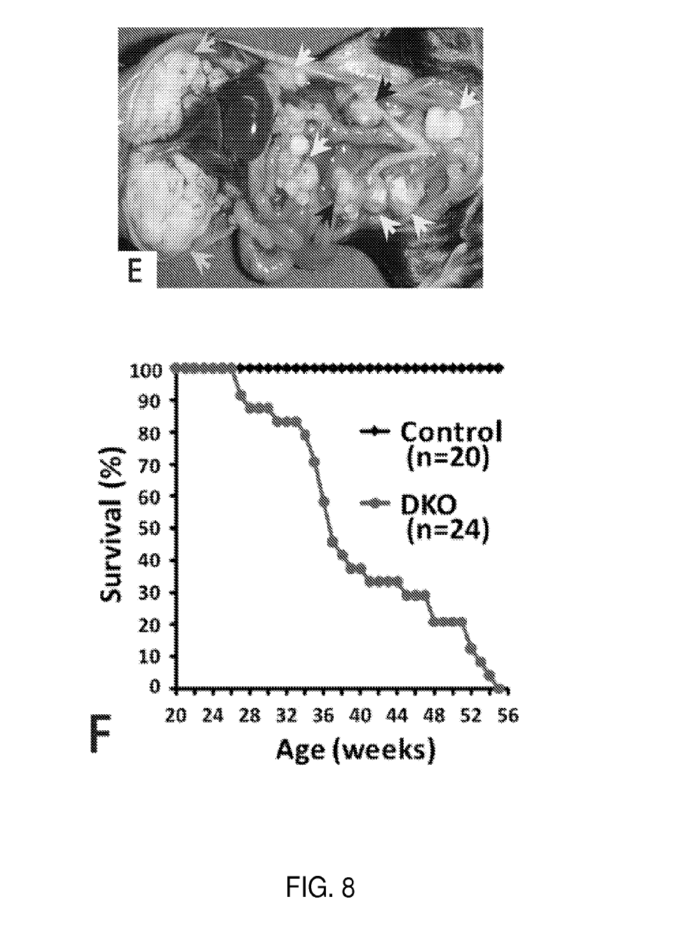

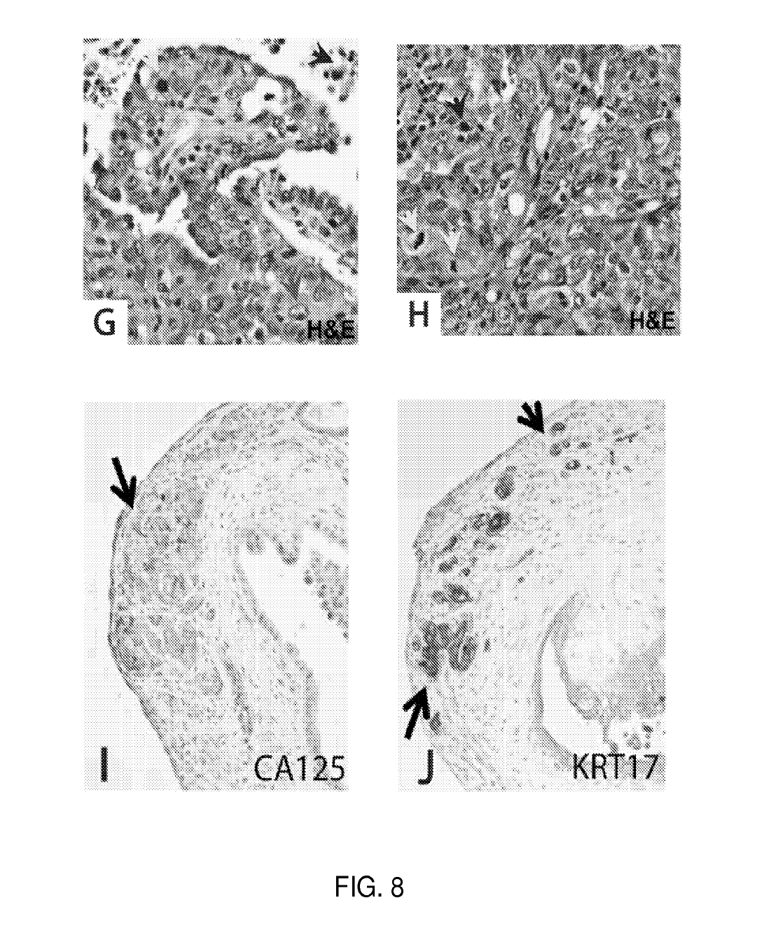

Cancer is a major cause of morbidity and mortality in humans, and ovarian cancer is the fifth most common cause of cancer-related death in women (Cho and Shih, 2009). Mutations in the p53 gene were first identified in ovarian cancers in 1991 and are frequently observed (Integrated genomic analysis of ovarian canrcinoma. Nature. 2011; 474(7353):609-15; Marks et al., 1991; Okamoto et al., 1991). The PI3K/RAS pathway, which is activated in 45% of ovarian cancers, is also activated by cisplatin, preventing apoptosis and leading to chemotherapeutic resistance (Bast et al., 2009; Peng et al., 2010). MiRNAs and their biosynthetic enzymes are altered in a broad range of cancers, including ovarian cancer, and define molecular signatures useful for cancer diagnosis or prognosis (Merritt et al., 2008; Du et al., 2005; Kumar et al., 2007; Esquela-Kerscher et al., 2006). In serous ovarian cancers, higher levels of the mRNAs encoding the miRNA biosynthesis enzymes, DICER and DROSHA, correlate with increased patient survival (Merritt et al., 2008). PTEN and DICER show frequent copy number losses (FIG. 6A). Based on these studies, it was considered that DKO of DICER and PTEN in the female reproductive tract would promote serous carcinomas. Consistent with this, 100% of DKO mice with conditional deletion of DICER and PTEN using AMHR2-Cre develop serous carcinomas that arise not in the ovary but the fallopian tube (FIG. 3). Likewise, women with familial ovarian cancer syndrome and who had prophylactic bilateral salpingo-oophorectomies are observed to have serous adenocarcinoma in their fallopian tubes but not in their ovaries (Medeiros et al., 2006), and “primary” high-grade serous carcinoma of the ovary and peritoneum are metastases from occult primary cancers that arise in the fallopian tube (Cho and Shih, 2009; Crum et al., 2007, Seidman et al., 2010; Marquez et al., 2005; Kindelberger et al., 2007). Mice with single KO of DICER (Nagaraja et al., 2008) or PTEN (Fan et al., 2009) show no cancers in the reproductive tract. The cancers of the fallopian tube in the DKO mice begin at ˜4-5 months, spread to the ovary, and metastasize to the peritoneum and induce ascites and 100% death by 55 weeks (FIG. 8). Histologically and immunologically, the fallopian tube primary cancers (n=23) and metastatic lesions (n=10) are typical of high-grade serous carcinomas that are observed in women (described in detail in FIG. 8). The primary fallopian tube cancers that arise in the stromal compartment are positive for epithelial markers [i.e, CA125, E-cadherin, and cytokeratins (KRT) 8, 14, and 17], even in the earliest cancer lesions (FIG. 3I-J). Although epithelial-to-mesenchymal (EMT) is common for cancers (Polyak and Weinberg, 2009), the fallopian tube serous carcinomas arise via a mesenchymal-to-epithelial transition (MET), such as from a mesenchymal stem cell that can differentiate along an epithelial lineage, in specific embodiments.

The mouse high-grade serous carcinomas recapitulate many aspects of serous carcinomas in women. Besides the histological findings (FIG. 8), the presence of cancer in the mice is grossly silent until metastasis occurs. The cancers metastasize to the peritoneum overlying the diaphragm in mice (FIG. 8E), a structure analogous to the human omentum, a site of common metastasis in women with ovarian cancer. To further confirm molecular similarities between the mouse and human cancers, gene set enrichment analysis (GSEA) was performed of the mouse DKO serous carcinoma dataset and the human TCGA serous ovarian cancer dataset (¹. GSEA and classical Spearman's rank sum statistic are rank-based methods that can detect modest but coordinate expression changes in groups of functionally related genes (Mootha et al., 2003; Subramanian et al., 2005). GSEA and classical Spearman's rank sum statistical analysis of the microarray data for the human serous cancers from TCGA (Integrated genomic analysis of ovarian canrcinoma. Nature. 2011; 474(7353):609-15) and the mouse DKO serous cancers reveal high statistically significant similarities. Genes differentially expressed in the mouse model tumors reflect corresponding patterns observed in human serous ovarian tumors. By GSEA, genes upregulated >2-fold in the human datasets were also statistically upregulated >2-fold in the mouse dataset (normalized enrichment score=4.8; P<0.001). Using Spearman's rank sum statistic to analyze the datasets, high statistical significance (Spearman's rank sum=11.1; P<0.00001) was obtained. Alternatively, using GSEA genes downregulated >2-fold in the human datasets were also statistically downregulated >2-fold in the mouse dataset (normalized enrichment score=−7.3; P<0.001). Likewise, Spearman's rank sum statistic analysis of the datasets produced high statistical significance (Spearman's rank sum=−15.7; P<0.00001). Thus, by global analysis of gene expression profiles, one can conclude that the mouse fallopian tube serous carcinomas share widespread similarities with the human serous ovarian cancers.

Using next generation sequencing, the expression of all miRNAs in the fallopian tube were analyzed, and it was discovered that miR-34c is the highest non-let-7 miRNA (4.1% of the miRNAs; FIG. 6B). P53 activates the three miR-34 family members, and miR-34 overexpression induces cell cycle arrest or apoptosis depending on the cellular context (He et al., 2007; Corney et al., 2007; Bommer et al., 2007; Raver-Shapira et al., 2007; Chang et al., 2007; Tarasov et al., 2007). Reduced miR-34 expression has been reported in ovarian cancer (Corney et al., 2007′ Corney et al., 2010; Lee et al., 2009), and methylation at the miR-34a and miR-34b/c loci was observed in 27% (8 of 30) and 47% (14 of 30) of ovarian cancer samples, respectively (Corney et al., 2010). Using miRNA arrays, miR-34c levels were found to be the sole independent predictor of recurrence-free survival for serous ovarian cancer (Lee et al., 2009). miR-34c levels are decreased 83-fold in human serous ovarian carcinomas (n=14) compared with fallopian tube (n=6)(p<0.005). In summary, the inventors have created a unique mouse that models serous epithelial carcinoma, the most common “ovarian” cancer in women. These observations support a novel paradigm for understanding the initiation and progression of high-grade serous carcinoma in vivo and the roles of miR-34c and downstream factors in the therapeutic responses of these cancers.

There are at least several aspects of this proposal that are innovative as follows: i) the first in vivo model (i.e., DICER/PTEN DKO mice) was generated that recapitulates many of the aspects of serous ovarian carcinomas, the most deadly cancer of the reproductive tract in women; ii) these DKO mice genetically define the fallopian tube as at least one of the sites of origin of high-grade serous “ovarian” cancer; iii) cells in the stroma of the fallopian tube undergo a mesenchymal-to-epithelial transition (MET) during initial formation of the serous carcinomas, indicating that a uterine mesenchymal stem cell not only is capable of playing an important role in normal maintenance of the epithelium during regeneration but can also go awry and develop into lethal serous carcinomas; iv) the earliest fallopian tube cancers express mRNAs encoding known and novel proteins that can be tested as ovarian cancer biomarkers in women; and v) mouse and human serous carcinomas have abnormal expression of several cancer genes and pathways, and overexpression of miR-34c in cancer cell lines have profound effects on these pathways and the cell cycle.

### Identification of Some Conserved Serous Cancer Biomarkers

To identify biomarkers that are expressed in early fallopian tube serous carcinomas, the inventors performed Illumina gene expression analysis of well-delineated mouse fallopian tube carcinomas or human ovarian serous cancers compared to mouse and human fallopian tubes, respectively. The mouse serous carcinomas resemble human serous carcinoma at the molecular level, and many genes upregulated in human serous carcinomas are also highly expressed in the DKO mouse serous carcinomas. Bioinformatic analyses of nine of the 36 upregulated genes that encode secreted or transmembrane proteins are shown (Table 1).

The list shows several known important genes in serous ovarian cancer such as secreted phosphoprotein 1 (SPP1, highly expressed in mouse and human ovarian cancer), CA125 (MUC16), folate receptor 1 (FOLR1, a GPI-anchored protein that can be sloughed from the cell membrane), and chemokines such as CXCL9, CXCL10, and CCL8. Along with CA125 (Bast et al., 1998), SPP1 has been suggested as a putative serum biomarker that can detect early ovarian cancer with high sensitivity and specificity (Meinhold-Herlein et al., 2007). FOLR1 is highly expressed in 90% of women with epithelial ovarian cancer, and the overexpression of this receptor is associated with high grade and advanced stage (Kallie et al., 2008). In addition, the chemokine CCL8 has been detected in ascites cells of >85% of women with epithelial ovarian cancer (Milliken et al., 2002). The molecular similarity of the mouse and human serous cancers strengthens the conclusion that the fallopian tube has the ability to initiate and develop high-grade serous carcinoma. Because there are no highly predictive blood tests for serous ovarian cancer and no tests for detecting early serous carcinomas before they metastasize, this data is helpful to identify biomarkers useful as diagnostic tools in women.

Analysis of Conserved Cancer Pathways that are Responsive to miR-34c Delivery.

The inventors also compared the expression of genes and pathways that are altered in both the mouse PTEN/DICER DKO cancers and human high grade ovarian cancers. Activation of the PI3K pathway leads to upregulation and phosphorylation of several genes (Milliken et al., 2002), and the inventors confirmed the increased phosphorylation of AKT1, PRAS40, and EIF4EBP1 (eukaryotic translation initiation factor 4E binding protein 1) and the dramatic upregulation of survivin (BIRC5) and stathmin (STMN1) by Western blot analysis (FIG. 4). STMN1 (stathmin) is a downstream target of PI3K involved in cytoskeletal reorganization (Salvesen et al., 2009). Phosphorylation of EIF4EBP1 would prevent its ability to bind and inhibit EIF4E, permitting increased translation initiation and protein synthesis (FIG. 2).

Well-known as an apoptosis inhibitor, survivin has been identified to contribute to nearly every aspect of cancer from onset to outcome (Altieri, 2008; Martinelli et al., 2006). Nearly all serous, endometrioid, and poorly-differentiated ovarian cancers express surviving (Cohen et al., 2003), the levels of survivin correlate with poor prognostic parameters (Cohen et al., 2003), high levels of survivin are associated with taxol/platinum resistance (Zaffaroni et al., 2002), and women with PTEN-negative/survivin-positive ovarian cancers have the worst prognosis (Sui et al., 2006). Examination of the collection of human high-grade serous ovarian cancers and cell lines demonstrates significant survivin upregulation above baseline in 21 of 24 cases. YM155 is a small molecule transcriptional suppressor of survivin, is fairly well-tolerated, has significant effects on many human cancer cell lines, has a better tolerated safety profile than cisplatin and paclitaxel at nanomolar concentrations but does not cause cell death for non-cancerous human cells (Nakahara et al, 2011). Based on these findings, the inventors treated mouse serous cancer cell lines and the human serous cancer line OVCAR8 with YM155 or everolimus, an inhibitor of mTOR (FIG. 5A, B). The tumors are sensitive to nanomolar levels of YM155 and micromolar levels of everolimus. These findings indicate that YM155 and everolimus can be used to identify small molecules that synergize with the compounds to eradicate high-grade serous carcinomas in women.

To further understand the molecular changes that contribute to high-grade serous carcinomas, the inventors searched for key cell cycle and survival pathway genes. The mouse and human serous adenocarcinomas show increased expression of several chromosomal passenger complex (CPC) protein mRNAs and proteins including survivin (increased in mouse tumors but almost undetectable in fallopian tubes), inner centromere protein (INCENP), and cell division cycle associated 8 (CDCA8) (FIGS. 2 and 4). In cancers, CPC requires each of the proteins for appropriate cellular localization and function of the other components. Activation of the PI3K pathway also leads to an elevation in MDM2 protein levels and significant increases in the mRNA encoding PDZ-binding kinase (PBK), proteins that bind and destabilize p53 (Haupt et al., 1997; Honda et al., 1997; Hu et al., 2010; Aksamitiene et al., 2010).

To characterize the roles of miR-34c in regulating major cancer and cell cycle pathways, mimics were transfected into three independent PTEN/DICER DKO primary serous cancer cell lines and the human OVCAR8 serous cancer cell line and assayed for cell viability and cell proliferation (FIG. 5C, D) and microarray analysis was performed, with followup QPCR confirmation. Delivery of miR-34c mimic to the mouse and human serous cancer cell lines has a dramatic effect on cell viability and proliferation. The effects were examined of miR-34c on major pathway genes that contain miR-34c binding sites in their 3′UTR sequences as well as pathways in which these proteins lie. Direct targets of miR-34c, minichromosome maintenance complex component 5 (MCM5), cell division cycle 6 (CDC6), cyclin E2 (CCNE2), and CDCA8, are downregulated 4.6-18.7-fold upon delivery of miR-34c as well as other key cellular genes including DNA topoisomerase 2a (TOP2A), which is upregulated in mouse and human serous cancers but suppressed 11.7-fold by the miR-34c mimic (Table 2). These findings are especially relevant to cancer treatment since mutations in TOP2A are associated with drug resistance, and TOP2A is the target for several chemotherapeutic agents. In contrast, murine retrovirus integration site 1 (MRVI1) homolog, myeloid leukemia tumor suppressor, is upregulated 10.2-fold.

TABLE 2
Major pathway genes altered in mouse and human serous
carcinomas and regulated in response to miR-34c delivery.
Asterisks denote genes with predicted miR-34c binding
sites in their 3′ UTR sequences.

| | Fold Change | | |
|---|---|---|---|
| | | | miR- |
| | Mouse | Human | 34c |
| Gene | cancer: FT | cancer: FT | mimic |
| Survivin | 19.4 | 5.4 | −4.8 |
| INCENP | 6.0 | 2.2 | −4.5 |
| CDCA8* | 9.3 | 1.7 | −9.6 |
| MCM5* | 12.3 | 3.8 | −18.7 |
| CDT1 | 6.3 | 5.8 | −6.3 |
| CDC6* | 5.8 | 1.2 | −5.5 |
| TOP2A | 13.5 | 4.2 | −11.7 |
| MDM2 | 1.7 | 1.3 | 1.0 |
| PBK | 17.2 | 3.8 | −5.7 |
| CCNE2* | 3.5 | 4.5 | −4.6 |
| CDK2 | 1.9 | 8.1 | −3.4 |
| MRVI1 | −15.7 | −4.9 | 10.2 |

These findings allow one to define the relevance of several key growth, proliferation, and survival pathways to cancer and develop a model for the involvement of miR-34c and these proteins in high-grade serous cancer (FIG. 7). Thus, miR-34c can directly alter apoptotic, DNA replication, and G1 cell cycle pathways in high-grade serous adenocarcinoma. To further test the model and the important roles of miR-34c downstream of p53, the inventors mated DICER^{Flox} and PTEN^{Flox} mice to p53-R172H^{LSL/LSL} mice (Olive et al., 2004) (i.e., Li-Fraumeni mutant mice) and created DICER/PTEN/p53-R172H conditional triple mutant mice using AMHR2-Cre. These triple mutant mice develop identical high-grade serous fallopian tube cancers as the DICER/PTEN DKO mice. Thus, the presence of the mutant p53-R172H allele does not alter the initiation or progression of the cancers, further indicating that p53 acts upstream of miR-34c and other genes and pathways that have been uncovered as embodiments of the invention. Because 70% of ovarian cancer deaths are observed in women with serous cancers (Koonings et al., 1989; Jemal et al., 2009), studies with these mice and mouse and human high-grade serous carcinoma cell lines will provide innovative treatments for the majority of women who die from this horrible disease.

### Example 5

### Defining the Roles of Mir-34C in Serous Cancer Initiation and Progression

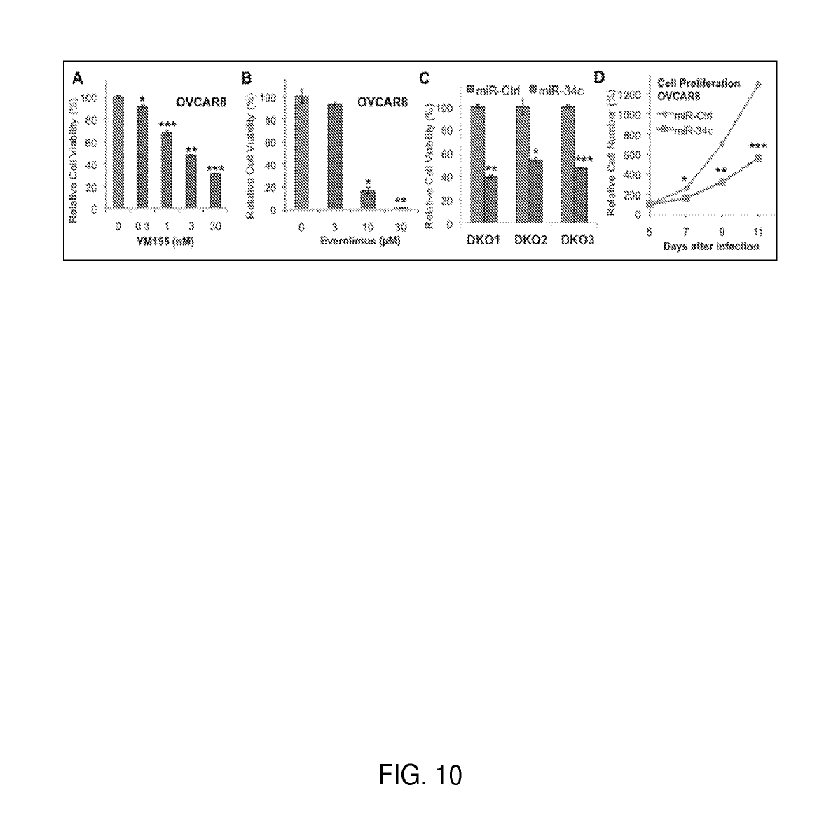

The inventors have shown that miR-34c is the most prominent non-let-7 family miRNA in the fallopian tube, is commonly lost in high-grade serous ovarian cancers, and is suppressed 83-fold in high-grade serous cancers (FIG. 6). Consistent with literature that shows that miR-34 family members function downstream of p53 and the roles of miR-34c in ovarian cancer (He et al., 2007; Corney et al., 2007; Bommer et al., 2007; Raver-Shapira et al., 2007; Chang et al., 2007; Tarasov et al., 2007; Polyak and Weinberg, 2009), the inventors have demonstrated that delivery of miR-34c mimics to DICER/PTEN DKO primary serous cancer cell lines or lentiviral delivery of miR-34c to human serous ovarian cancer cell lines has dramatic effects on the cell cycle and key direct and indirect targets of miR-34c (FIG. 10B, C). In this Example, it is described how one can first analyze the effects of miR-34c on the gene expression changes in established human serous adenocarcinoma cell lines and then use mouse genetics to characterize that miR-34c is the sole miRNA needed for serous cancer initiation and progression in vivo by creating miR-34b/c/PTEN DKO mice.

Exemplary Experimental Design:

Analyze the Human Gene Expression Changes Induced by miR-34c Delivery.

Several assays have been performed to show that miR-34c causes dramatic effects on the cell cycle and cell survival. To further understand the evolutionarily conserved roles of miR-34c as a putative tumor suppressor for serous carcinomas, one can transfect the miR-34c and control mimics into three exemplary independent serous carcinoma cell lines that display essentially no detectable miR-34c (i.e., OVCAR5, SKOV3, and HEY), collect mRNA 48 hours after transfection, and then perform Illumina Human WG-6 v3 Expression BeadChip. Statistical and clustering analysis of the microarray data is performed using GeneSpring software, the control mimic group versus the miR-34c mimic group are analyzed, and the data are compared to the data from the miR-34c mimic transfections into the mouse DKO cancer cell lines. In addition to these analyses, one can also use SigTerms software (Creighton et al., 2008). SigTerms evaluates functional miRNA:mRNA pairs to make predictions about direct miRNA targets versus indirect effects upon transfection of the miRNA mimic (Creighton et al., 2010; Nagaraja et al., 2010; Hawkins et al., 2011).

Genetically Analyze the Requirement for miR-34c in Serous Carcinoma In Vivo.

To characterize that miR-34c is a major miRNA downstream of p53 and functions as a suppressor of high-grade serous carcinoma formation in the DICER/PTEN DKO mice, one can produce mice that lack both miR-34b/c (mouse chromosome 9) and PTEN (mouse chromosome 19) in the female reproductive tract. The miR-34b/c mutant mice are obtained. One can first produce miR-34b/c^{+/-} PTEN^{Flox/+} and miR-34b/c^{+/-} PTEN^{Flox/+} Amhr2-Cre⁺ males and females, and these mice are intercrossed to generate miR-34b/c PTEN^{Flox/Flox} female controls (i.e., homozygous null at the miR-34b/c locus but effectively wild-type at the PTEN locus; herein called control mice) and miR-34b/c^{-/-} PTEN^{Flox/Flox} Amhr2-Cre⁺ (i.e., homozygous null at the miR-34b/c locus and Cre inducible deletion of PTEN in the fallopian tube stroma; herein called miR-34b/c/PTEN DKO mice). Because miR-34b/c^{-/-} mice are viable, DKO mice are easily produced. The DKO and control females are analyzed in the following experiments:

1. Perform Long-Term Cancer Analysis.

miR-34b/c/PTEN DKO and controls (30 females of each genotype) are caged for long-term tumor analysis. Mice are weighed weekly to study tumor development. Because DICER/PTEN DKO mice develop ascites (due to metastatic spread of the cancers), weekly analysis is predictive of ascites and tumor development and allows one to determine when the mice have reached end stage metastasis development. Because 100% of the DICER/PTEN DKO mice die before 13 months, the miR-34b/c/PTEN DKO and control mice are caged for up to two years and survival curves created. All end-stage cancer mice are euthanized and necropsied, and primary and metastatic tumors and ascites collected for histologic and RNA/protein analysis and to generate cell lines.

2. Analyze Mice and Tumors by Gross and Histological Analysis at Earlier Timepoints.

Besides the tumors from the end stage mice, ten miR-34b/c/PTEN DKO and controls are euthanized and necropsied at earlier timepoints (e.g., 6 months) prior to ascites formation. Reproductive tracts are grossly and histologically analyzed to identify the early stages of cancer development and to compare the findings to the DICER/PTEN DKO mice for any histologic changes in the cancers. Early fallopian tube cancers are collected for mRNA analysis.

3. Perform Molecular Analysis.

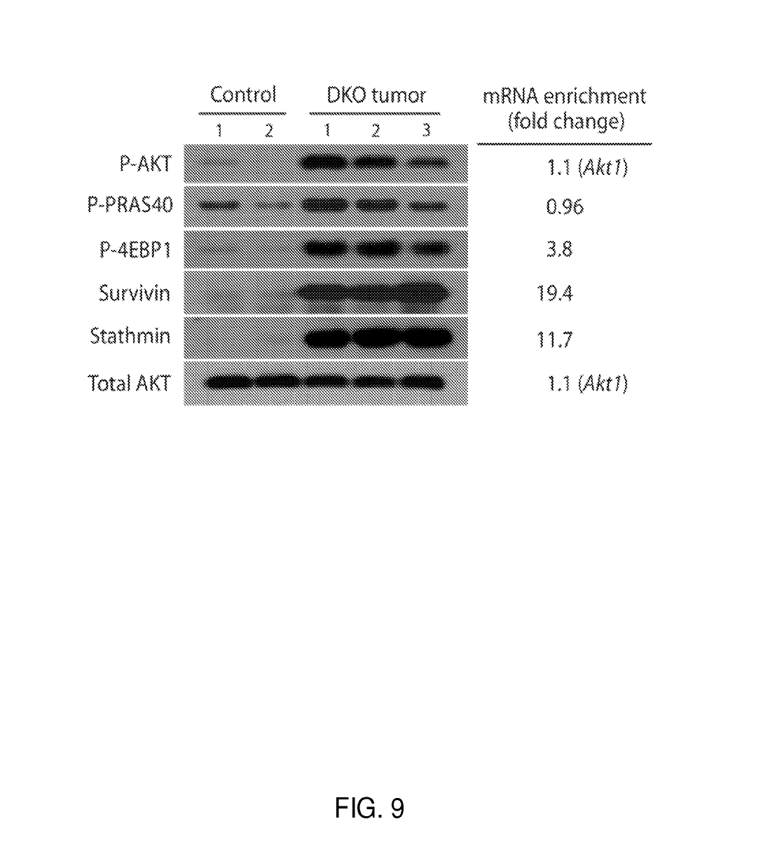

If fallopian tube serous carcinomas form in miR-34b/c/PTEN DKOs, one can use Illumina mRNA BeadChips to analyze the molecular defects in the miR-34b/c/PTEN DKO primary cancers versus control fallopian tubes and compare these data to Illumina analyses of DICER/PTEN DKO tumors Immunohistochemistry is performed with antibodies to KRT14, KRT17, and KRT8 as shown in FIG. 8 to confirm similar mesenchymal origins of the miR-34b/c/PTEN DKO cancers. Western blot analysis of key PI3K pathway proteins are also performed as shown in FIG. 9.

In some embodiments of the invention, miR-34b/c/PTEN DKO females develop serous carcinomas in their fallopian tubes but at a slower rate compared to the DICER/PTEN DKO mice because of the continued presence of additional tumor suppressor miRNAs in the fallopian tube mesenchymal cells that give rise to the cancers. However, if the primary tumors arise at a slower rate, the rate at which these cancers metastasize is also likely to be slower, and the miR-34b/c/PTEN DKO mice may reach their normal lifespan and begin to die of causes unrelated to the cancer metastasis. One can consider that at least the primary cancers are histologically identical to the DICER/PTEN DKO cancers, in certain embodiments. Gene expression changes may be different between the miR-34b/c/PTEN DKO and DICER/PTEN DKO tumors because of additional miRNAs in the miR-34b/c/PTEN DKO tumors, in some cases. However, the major pathways that are causal for tumor development continue to be aberrant, and this helps to further define the key pathways that give rise to the cancers, in specific embodiments. Thus, microarray analyses generate molecular insights into high-grade serous cancer development in women.

In alternative embodiments wherein the miR-34b/c/PTEN DKO mice fail to develop serous cancers, one can interrogate the individual and synergistic roles of additional miRNAs in these ovarian cancers including miR-31 and miR-100 (Creighton et al., 2010; Nagaraja et al., 2010). For these alternative studies, one can deliver the most highly expressed miRNAs alone or in combination with the miR-34c mimic and evaluate cell proliferation and apoptosis similar to the initial data. One can alternatively choose to use the Dharmacon mimic library that contains all of the miRNA mimics based on version 16.0 of miRBase. Depending on these findings, one can cross the PTEN mutant mice with additional miRNA mutant mice that are available or are created and/or create triple KO mice based on the results of the miRNA mixing studies described herein. Lastly, one could also use RNA-Seq as an alternative strategy for defining novel transcripts regulated by miR-34c in human cancer cells similar to the studies described elsewhere herein.

### Example 6

### Uncover Unique Therapeutic Approaches to Treat Serous Carcinomas

Many ovarian cancer patients show initial treatment successes, and debulking surgery helps improve survival. However, most of these patients show recurrence, and the majority die of their cancer. While better protocols are being developed to fight this disorder, additional molecular knowledge is required to develop state-of-the-art treatment strategies to eradicate the recurrent chemoresistant cancer cells. In some embodiments of the invention, DICER/PTEN DKO cancer cell lines, human serous cancer cell lines, and DKO tumor-prone mice are excellent in vitro and in vivo models for characterizing novel cancer therapies to eradicate high-grade serous cancer cells in women. Based on the initial data, the importance of survivin overexpression in ovarian cancer, and its association with platinum and taxol chemotherapeutic resistance (Sui et al., 2006; Cohen et al., 2003; Zaffaroni et al., 2002), one can develop in vitro assay strategies to identify synergistic combinations of drugs (e.g., YM155 and everolimus) and miRNAs (e.g., miR-34c) that eradicate the cancer cells in vitro and can be tested for their efficacy in vivo. One can also utilize gene expression profiles and Connectivity Map (CMap) tools to find additional drugs that can act synergistically to kill ovarian cancer cells but spare normal cells.

Experimental Design:

a. Develop In Vitro Screens with Small Molecules and miR-34c Mimics to Identify Combinatorial Strategies to Eradicate High-Grade Serous Carcinomas.

One can develop synthetic lethal screens to identify the best combination small molecule/miRNA strategies to eradicate serous carcinomas. Because the high-grade serous carcinoma in mice and women are nearly identical and both express high levels of survivin and other key cell cycle pathways, one can utilize mouse and human cancer cells for in vitro testing. Because the cancer cells are sensitive to YM155, everolimus, and miR-34c (FIG. 10), one can perform both directed screens and unbiased approaches to find small molecules that synergize with these drugs/mimic The assays are performed in 96-well format in triplicate in the presence or absence of YM155, everolimus, and/or miR-34c mimic using a mouse serous cancer cell line and a human serous cancer cell line (OVCAR8). Because the human cancer cell lines have been passaged for many generations and may not represent the original cancers, a goal is to find synergies that work effectively for both the mouse and human cancers and would be more universally effective for all serous cancers that arise in women. These assays can utilize a Beckman-Coulter Biomek TX Automation Workstation. One can initially start on screens with 2600 off patent drugs for these cell-based assays. Cell viability is the readout, because the goal is to kill the cancer cells not just slow their growth, in certain embodiments, although in certain aspects one slows their growth.

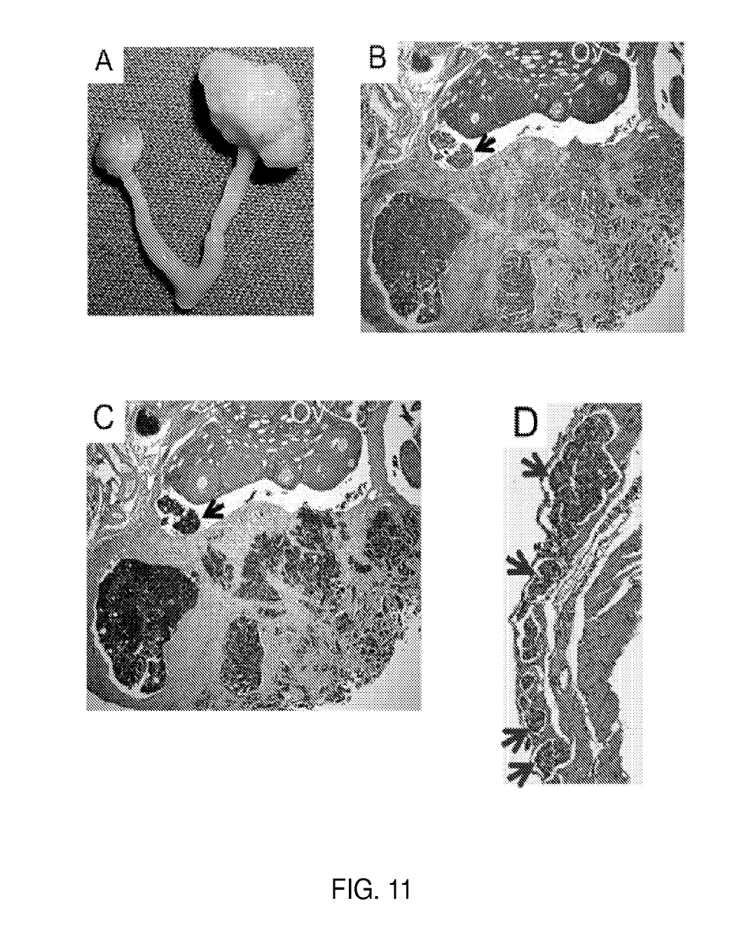

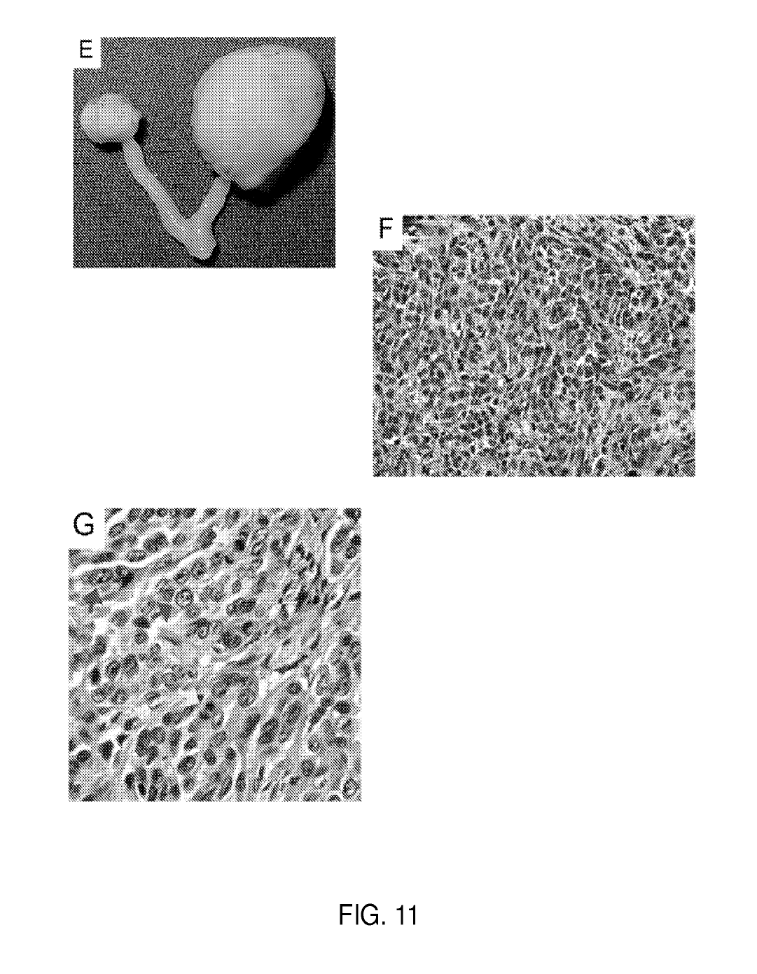

Drugs that appear to synergize with YM155, everolimus, or miR-34c are tested further in secondary validation assays in varying doses using additional human and mouse serous cancer cell lines. The best combination of small molecules/miRNA mimic subsequently are tested for their efficacy in vivo using DKO mice with metastatic cancers and/or SCID mice injected with established serous cancer cell lines. One can also utilize human cancer cell lines that are established from ascites of women with metastatic high-grade serous ovarian cancer (FIG. 11). These human ovarian cancer cell lines phenocopy the spread of ovarian cancer in women and mice and home in and proliferate around the mouse ovaries and the diaphragm when injected into SCID mice (FIG. 11). For these studies with the mouse DKO lines, the human established cancer lines, and the newly created high-grade cancer lines, one can inject 2×10⁶ cancer cells/mouse and include 5-10 mice/treatment. These in vivo studies can recapitulate the conditions that yield the most effective in vitro apoptotic effect (whether it be a single agent or a combination treatment). The treatments can begin one-week post-inoculation of the cells and can continue for three additional weeks. During the course of these treatments, one can measure body weight and abdominal circumference of the mice every three days, and at the end of the study, one can measure intraperitoneal tumor mass and ascites fluid.

To understand the molecular synergies between drugs/treatments that are most effective, one can treat the human ovarian cancer cells under the most effective single and combination drug treatment protocol, isolate RNA at an early time, and perform additional Illumina gene expression analysis. In particular, the gene expression changes upon treatment of ovarian cancer cells with YM155 are interesting. Because YM155 is believed to suppress survivin gene expression, a time course analysis of the cascade of events that occur upon treatment at 0, 1, 5, 12, 24, and 48 hours post-delivery and follow-up bioinformatics is beneficial to further characterization of how YM155 and survivin function in treating cancer. All of this data is examined using standard GeneSpring analysis as well as Connectivity Map (CMap) tools as described below.

One can identify effective combinations of small molecule and miR-34c mimic that eradicate serous cancer cells in vitro. Accordingly, some of these approaches are optimized in vitro and used to treat mice bearing these cancers in vivo. The success in the treatment of cancers in the mouse model is a key step toward development of novel strategies for the therapeutic intervention of human serous “ovarian” cancer.

b. Use CMap Tools to Discover Alternative or Synergistic Strategies to Treat Ovarian Cancers.

Although delivery of miR-34c mimics is a useful therapy for treatment of women with epithelial cancers, one can identify small molecules that can mimic the effects of miR-34c. One can use CMap tools and gene expression data (Lamb et al., 2006) to establish embodiments regarding pathways in serous carcinomas that are therapeutic targets. CMap is comprised of 453 gene expression profiles of cell lines obtained by treatment in vitro with 164 different, bioactive small molecules. CMap uses this expression data and pattern-matching algorithms to discover functional connections between drugs, genes, and diseases (Lamb et al., 2006). The inventors queried the CMap database with the mRNA expression data for the miR-34c mimic study to predict therapeutic responses of the serous carcinomas to specific pharmacologic agents for ovarian cancer therapy. At the top of the list were several drugs with anti-cancer and pro-apoptotic effects including withaferin A (a steroidal lactone) (Hahm et al., 2011), parthenolide (a sesquiterpene lactone)(Mathema et al., 2011), vorinostat (an HDAC inhibitor) (Modesitt et al., 2010), and scriptaid (an HDAC inhibitor) (Takai et al., 2006). One can test the efficacy of these drugs alone and in combination with small molecules that are identified to synergize with miR-34c mimics as well as YM155 and everolimus. In addition, for specific combinations of small molecules that are efficacious, one can perform drug treatments in triplicate, isolate RNA, and carry out Illumina gene expression arrays as described in the previous section and then perform cMap analyses again. These considerations are directly tested in vitro using the assays described elsewhere herein and direct one toward the development of additional combinatorial drug treatments upon identification of small molecule pharmacologic agents.

Thus, embodiments of the invention include combinatorial strategies to therapeutically kill cancer cells in vitro by targeting key pathways through delivery of small molecules and/or miR-34c identified through the screens and CMap analysis. These studies allow one to develop novel strategies to inhibit high-grade serous carcinoma in patients in vivo. Depending on the pathways altered in the primary and metastatic cancers, additional in vitro and in vivo therapeutic approaches (alone and/or in combination with the above treatments) are performed. Because FOLR1 is dramatically upregulated in high-grade ovarian cancer cells (Table 1) and FOLR1 is expressed in over 80% of serous ovarian cancers and at higher levels in high-grade and high-stage cancers (Kalli et al., 2008), there are many alternative strategies being developed to utilize the folate receptor as a means to more selectively treat ovarian cancer. For example, Farletuzumab (MORAb-003) is a monoclonal antibody to FOLR1 that is being used in clinical trials for ovarian cancer (Ledermann and Raja, 2010). Alternatively, delivery of folate-linked nanoparticles encapsulating paclitaxel and yittrium-90 enhances survival of mice with ovarian metastasis (Werner et al., 2011). Cationic folate-linked nanoparticles encapsulating siRNAs and plasmids are also being developed to treat cancer (Hattori, 2010). Long-term expression of shRNA in cancer cells using Minivector DNA has been demonstrated (Zhao et al., 2011) and there are also particle delivery strategies (Wang et al., 2010). In embodiments wherein drugs may be toxic to cells, an alternative is to deliver YM155 (or whichever drug) without encapsulation but encapsulate other more toxic drugs and/or Minivectors expressing shRNA to survivin or miR-34c that are to be delivered in combination. Also, the studies described herein uncovers genes that are involved in ovarian cancer metastasis and leads to development of alternative strategies that block cancer metastasis in vivo by specifically targeting pro-metastasis proteins.

### Example 7

### MIR-34C is a Tumor Suppressor MicroRNA in Dicer-Pten Double Knockout High-Grade Serous Carcinomas

MicroRNAs (miRNAs) are short non-coding RNAs that could have large-scale biological effects by directing gene regulation through translational repression and degradation of multiple complementary target mRNAs. Like other regulatory molecules, altered miRNA expression has been suggested to be involved in the formation of many human diseases, including ovarian cancer. The present invention encompasses a mouse model with high-grade serous carcinomas by conditionally deleting both Dicer (essential for microRNA biosynthesis) and Pten (a negative regulator of the PI3K pathway) in the female reproductive tract. Because of the fact that Pten knockout alone did not result in serous ovarian cancer, the significance of the impaired mature miRNA biosynthesis is highly emphasized in this Dicer-Pten double knockout (DKO) high grade-serous carcinomas, in certain embodiments. To define the specific miRNAs in this DKO mouse model, cell lines derived from primary ovarian tumors in the Dicer-Pten DKO mice were generated. Lack of Dicer makes the cell lines generated from these mice to be a valuable platform on functionally evaluating the significance of miRNAs in this model. The inventors delivered control miRNA, miR-31, miR-100, let-7b, and miR-34c mimic to Dicer-Pten DKO cell lines by transient transfection. There was a growth inhibitory effect of miR-34c that is accompanied with cell cycle arrest at G1 phase and induction of apoptosis. miR-34c is a direct transcriptional target of p53 whose mutation is the most frequent in human ovarian cancers. Using quantitative real-time PCR, miR-34c levels were extremely low in human serous adenocarcinomas compared with normal fallopian tube. Enforced expression of miR-34c in a human serous ovarian cancer cell line induced cell growth arrest further indicating that the data on miR-34c in the mouse model is translatable to women.

As indicated in FIG. 6B, let-7 family members and mir-34c are the most abundant miRNAs in mouse fallopian tubes.

Cancer cells generated from Dicer-Pten DKO primary ovarian tumors are epithelial cells, as indicated by immunofluorescence analysis of Keratin8 and Keratinl4 in Dicer-Pten DKO mouse cancer cell lines.

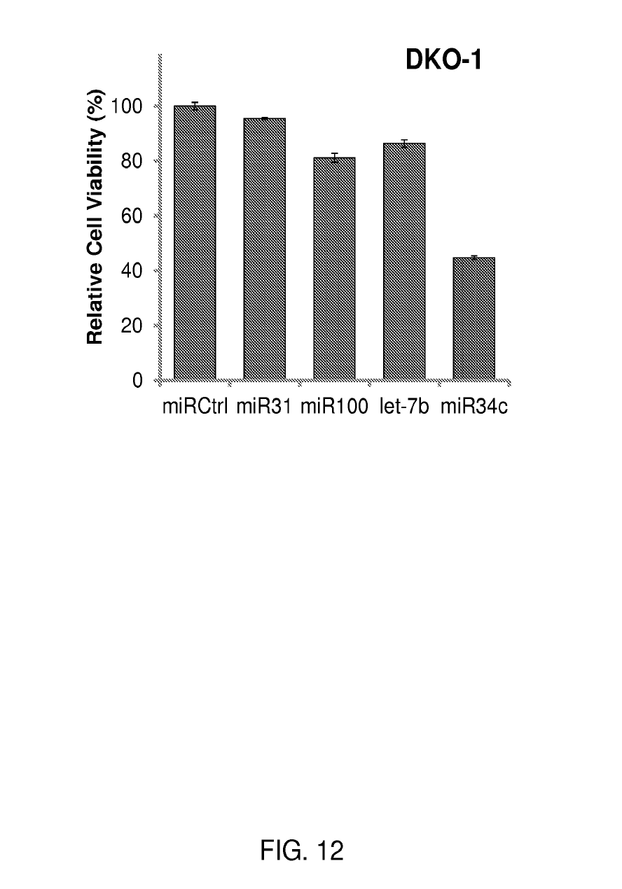

miRNA mimic initial screening reveals the significance of miR-34c in Dicer-Pten DKO mouse ovarian cancer, as shown in FIG. 12. Dicer-Pten DKO mouse cancer cell line DKO-1 were transfected with various miRNA mimics including miR-31, miR-100, let-7b, miR-34c or a miRNA control (miR-Ctrl). Cell viability was measured by ATP quantitation-based CellTiter-Glo assay 48 hours after transfection.

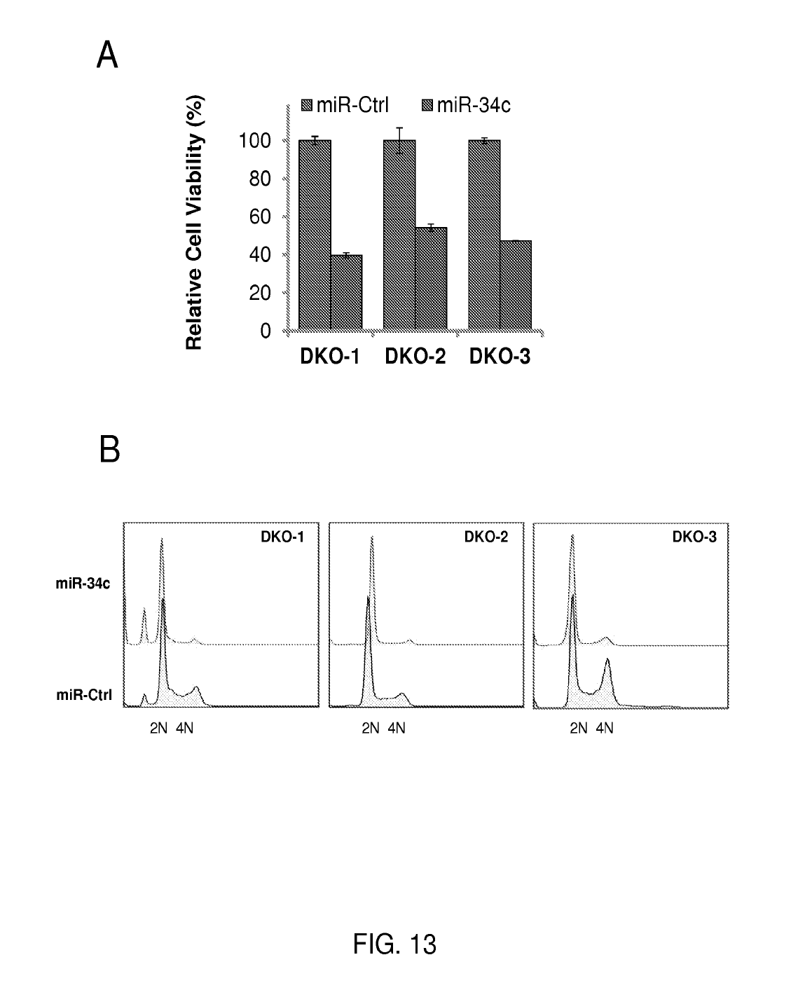

FIG. 13 shows that validated cell viability inhibitory effect of miR-34c is associated with cell cycle arrest in G1 phase. FIG. 13A is a cell viability assay. Dicer-Pten DKO mouse cancer cell lines were transfected with miR-34c followed by cell viability assay in 48 hours using CellTiter-Glo assay. FIG. 13B is cell cycle analysis. Forty-eight hours after miR-34c transfection, Dicer-Pten DKO mouse cancer cell lines were fixed in cold 70% ethanol and stained with PI. Cell cycle profiles were determined by flow cytometry and analyzed by flowjo.

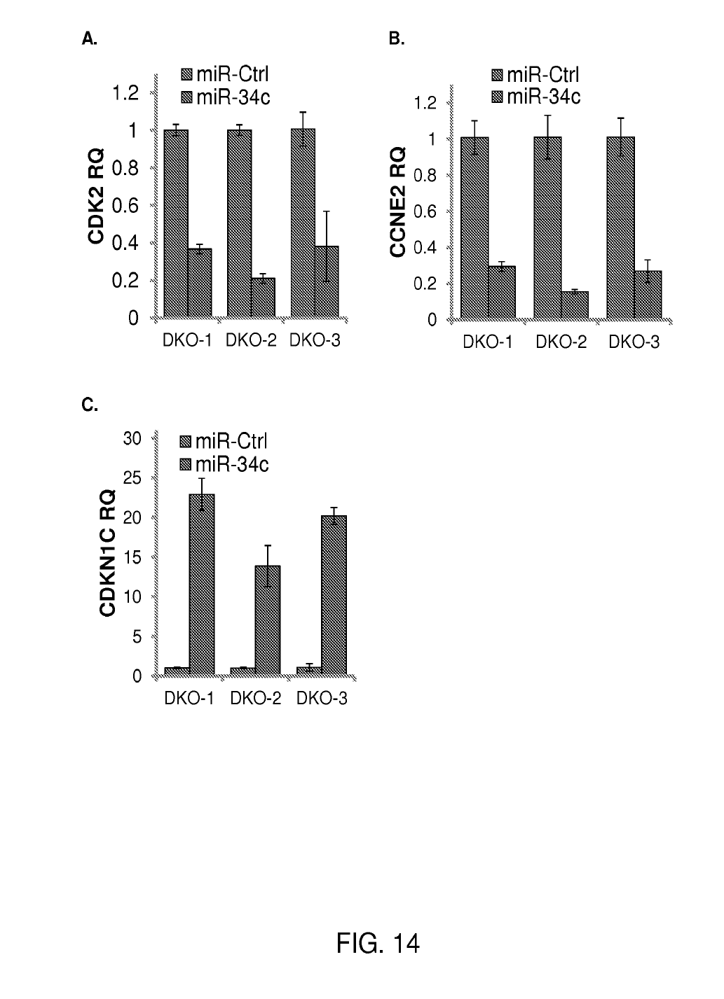

In FIG. 14, miR-34c inhibits cyclinE-CDK2 complex. Analysis of some of miR-34c downstream genes is shown. QPCR analysis showing relative quantity of CDK2 (A), CCNE2 (B) and CDKN1C(C) after miR-34c mimic transfection in Dicer-Pten DKO mouse cancer cell line.

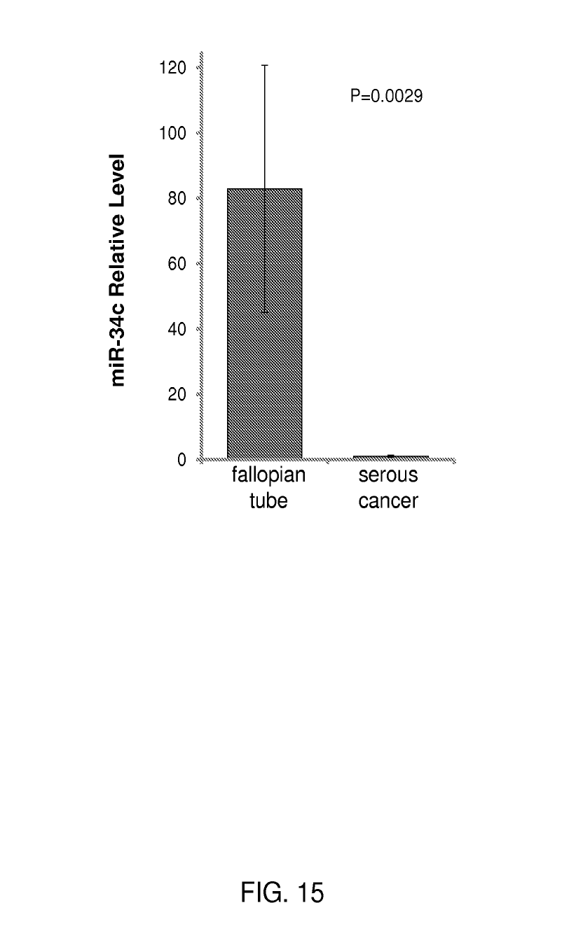

miR-34c levels were decreased 83-fold in human serous adenocarcinomas compared with fallopian tube (FIG. 15). In FIG. 15, 6 miR-34c Taqman QPCR is shown, wherein levels of mature mi-34c in normal human fallopian tube (n=6) and human serous ovarian cancers (n=14) were determined by quantitative PCR using commercially available Taqman probes with U6 snRNA as an internal standards for normalization.

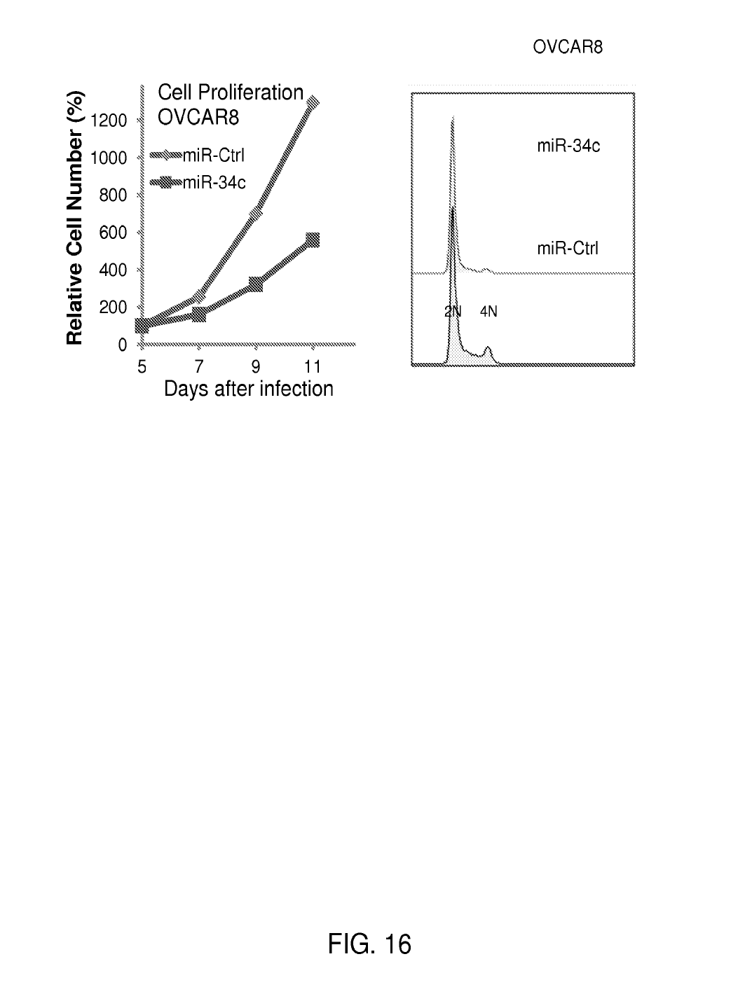

FIG. 16 shows similar effect of miR-34c in human serous ovarian cancer cell line. Therein, Lentiviral expression of miR-34c in OVCAR8 inhibits cell proliferation. In FIG. 16A, OVCAR8 infected with miR-34c or control lentivirus were seeded onto 96-well plates for proliferation assay measured by CellTiter-Glo assay. In FIG. 16B, five days after miR-34c or control lentivirus infection, cells were fixed in cold 70% ethanol and stained with PI. Cell cycle profiles were determined by flow cytometry and analyzed by flowjo.

### Example 8

### Ym155 and Parthenolide Synergize to Kill Ovarian Cancers and Pancreatic Cancers Through Endoplasmic Reticulum Stress and Reactive Oxygen Species Pathways

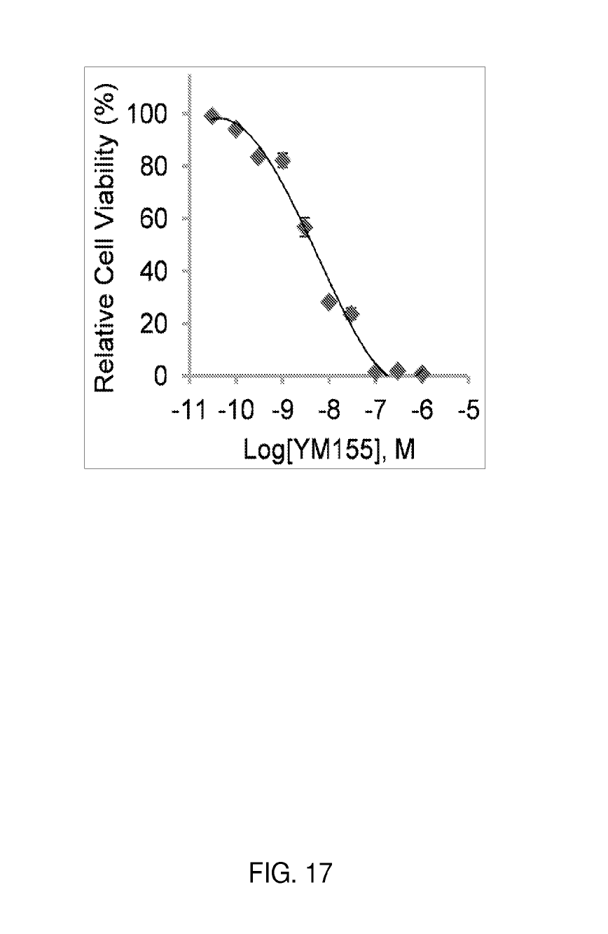

YM155, also known as sepantronium bromide, is a small, imidazolium-based compound that has potential anti-neoplastic activity due to its proapoptotic effect. YM155 was first identified in a survivin promoter-reporter screening assay for its suppression activity (Nakahara et al., 2007). Survivin is considered as one of the most cancer-associated proteins and is highly overexpressed in many human cancers but mostly suppressed in normal cells (Sah et al., 2006), making it an ideal target for cancer therapy. YM155 has subsequently been evaluated in many preclinical and clinical studies for its potential anti-cancer effect. Preclinical data has demonstrated that YM155 inhibited a wide variety of human cancer cell lines and xenograft models (Nakahara et al., 2011), and the inventors have confirmed the similar sensitivity of ovarian cancer and pancreatic cancer lines to YM155 in the low nanomolar concentration range (FIG. 17). In addition, YM155 has been administered to cancer patients in Phase I and Phase II clinical trials including prostate cancer, melanoma, non-small cell lung cancer, breast cancer, and lymphoma. Although data from these studies demonstrated favorable safety and tolerability of YM155, the single agent activity of this compound is modest or limited (Cheson et al., 2011; Giaccone et al., 2009; Lewis et al., 2011; Satoh et al., 2009; Tolcher et al., 2008; Tolcher et al., 2012). Although YM155 was initially discovered based on its ability to directly block expression of a survivin promoter-reporter construct, recent data (Glaros et al., 2012; Nakamura et al., 2012) has indicated that survivin is not the direct target of this compound. Studies reveal that YM155 acts via suppression of HSP72 genes and induction of an ER stress response pathway, indicating a novel mechanism of action of YM155.

Parthenolide, a sesquiterpene lactone, is the active ingredient in the medicinal herb, fever few. Parthenolide and its more water-soluble derivative, dimethylamino-parthenolide (DMAPT) are active in the micromolar range and alter several different pathways including activation of the NF-kB pathway. However, several studies suggest that parthenolide and other sesquiterpene lactones (DMAPT and costunolide) function to increase reactive oxygen species (ROS)(Guzman et al., 2005; Shanmugam et al., 2010; Yang et al., 2011). In embodiments of the invention, YM155 and parthenolide synergize via the ability of YM155 to target the ER stress pathway and parthenolide to increase ROS and contribute to cellular stress.

To characterize the early targets of YM155 in cancer cells, the ovarian cancer cell line OVCAR8 was treated acutely for 6 hours with YM155 (10 nM). Using Illumina gene expression analysis and QPCR followup, YM155-treated cells were compared with DMSO vehicle treated cells. Only 2 genes were statistically downregulated, 8 genes were upregulated >2-fold, and survivin was not significantly altered (Table 2). Five of the eight genes that were upregulated >2-fold are markers of the ER stress response (i.e., DDIT4, DDIT3, TRIB3, ATF3, and HERPUD1), indicating an induction of ER stress response by acute treatment with YM155. DDIT3 (a.k.a., CHOP) is a proapoptotic protein whose induction is a hallmark of ER stress induced apoptosis (Wang et al., 2010). The two genes dramatically downregulated in the list are HSPA1A and HSPA1B, which encode HSP72, a member of heat shock protein 70 family. HSP72 has strong cytoprotective effect by functioning as a molecular chaperone in cytosolic protein folding, transport, and degradation (Morimoto et al., 1997; Tavaria et al., 1996), and as an inhibitor of cellular apoptosis (Gamido et al., 2003; Samali and Orrenius, 1998). In addition, HSP72 interacts with ER stress sensor protein IRE1a thereby enhancing IREla-XBP1 signaling at the ER and promoting adaptation to ER stress and cell survival (Gupta et al., 2010). HSP72 is normally expressed at low levels, and is induced upon exposure to environmental stress to exert its cytoprotective effect. However, in cancer, HSP72 is constitutively expressed in many cancer cell lines and biopsies, and its expression correlates with poor prognosis (Gabai et al., 2009). HSP72 provides a selective advantage to cancer cells by suppressing default senescence via p53-dependent and p53-independent pathways (Yaglom et al., 2007). Thus, YM155 acutely suppresses HSPA1A and HSPA1B, indicating a novel mechanism which may contribute directly to the anti-cancer effect of YM155.

TABLE 2
HSP72 genes and ER stress related genes are altered in
OVCAR8 cells treated with YM155 for 6 hours. Survivin (BIRC5)
mRNA levels are not statistically altered by YM155 treatment.

| | | Fold Change | |
|---|---|---|---|
| | | YM155 | YM155 |
| | | (10 nM)/ | (30 nM)/ |
| | | DMSO | DMSO |
| Symbol | Gene Name | (Array) | (QPCR) |
| HSPA1A | Heat shock 70 kDa protein 1A | −2.6 | −7.5 |
| HSPA1B | Heat shock 70 kDa protein 1B | −2.6 | −4.4 |
| BIRC5 | Survivin | −1.3 | −1.1 |
| HERPUD1 | Homocysteine-inducible, ER | 2.2 | 3.1 |
| | stress- inducible, ubiquitin-like | | |
| | domain member 1 | | |
| ATF3 | Activating transcription factor 3 | 2.2 | 11.1 |
| TRIB3 | Tribbles homolog 3 | 2.6 | 2.0 |
| DDIT3 | DNA-damage-inducible transcript | 3.8 | 3.6 |
| | 3 | | |
| DDIT4 | DNA-damage-inducible transcript | 5.6 | 10.6 |
| | 4 | | |

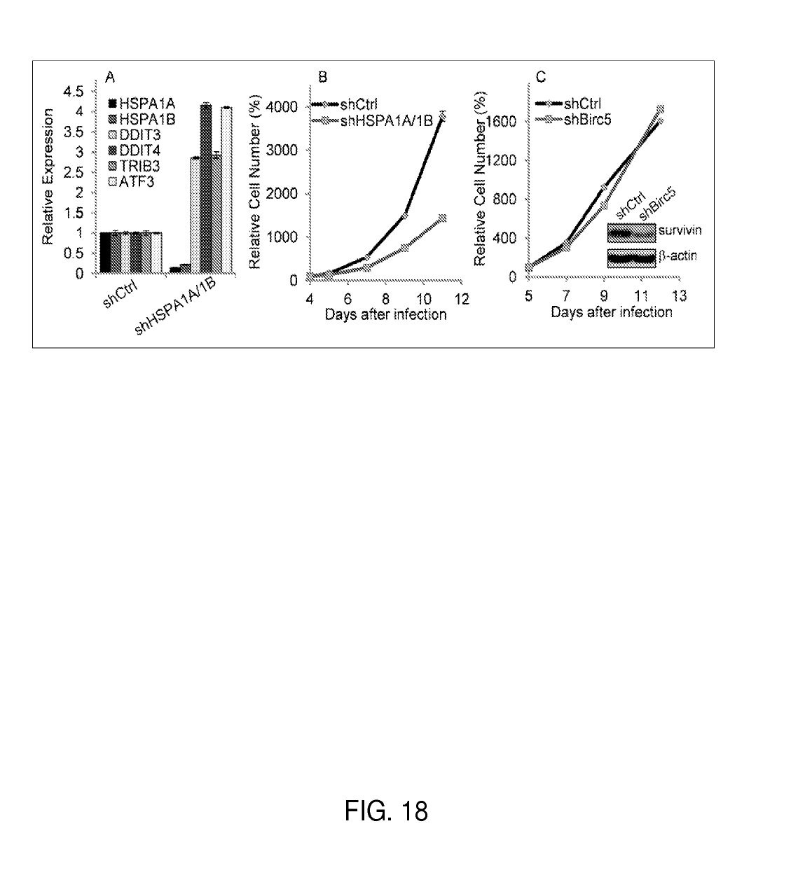

The effects of YM155 on suppression of the HSP72 genes and induction of the ER stress response were confirmed with three other human ovarian cancer cell lines (HEY, OV90, and SKOV3). To determine whether there is a functional connection between HSP72 depletion, induction of the ER stress response genes, and growth inhibitory effect of YM155 on ovarian cancer lines, HSPA1A and HSPA1B were knocked down using lentiviral delivered shRNA. As mentioned above, HSP72 interacts directly with ER stress sensor IRE1a thereby protecting cells from ER stress induced apoptosis (Gupta et al., 2010). Using QPCR, the data confirmed the efficiency of the HSPA1A and HSPA1B knockdown and demonstrated that the ER stress genes were significantly induced upon HSPA1A and HSPA1B knockdown (FIG. 18A). These findings indicate that HSP72 depletion can directly induce an ER stress response. Knockdown of HSPA1A and HSPA1B also significantly inhibited cancer cell proliferation (FIG. 18B), whereas knockdown of survivin (BIRC5) in ovarian cancer cells had no effect (FIG. 18C). In certain embodiments of the invention, survivin is not the target of YM155, and YM155 acts through a novel mechanism that includes suppression of HSP72 and induction of ER stress.

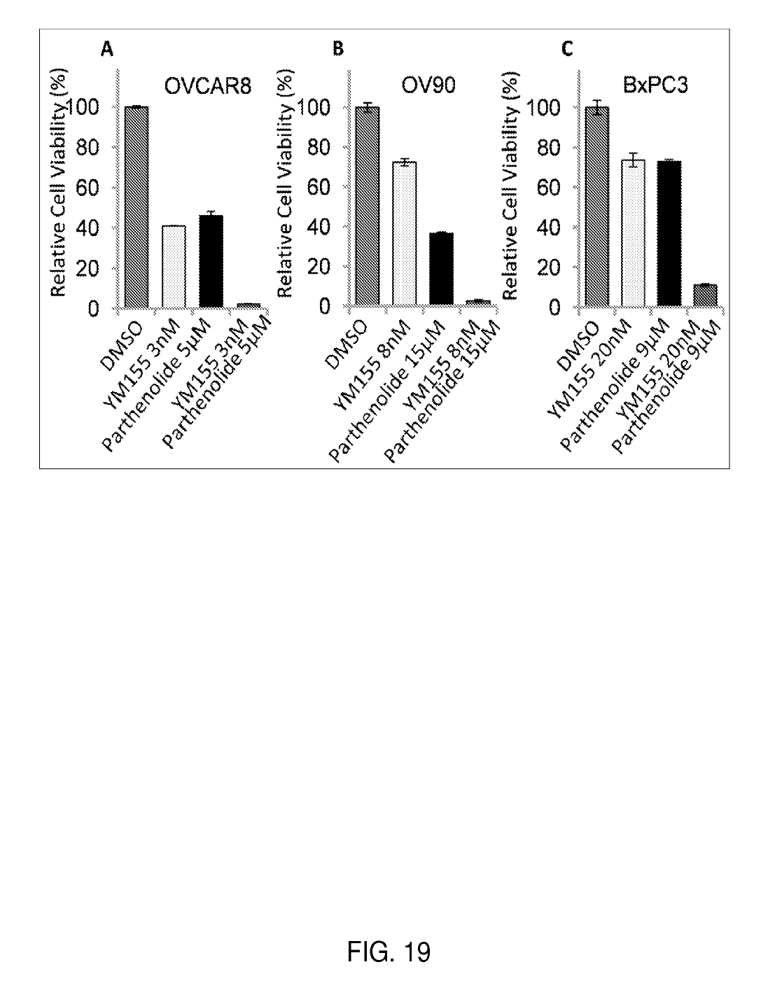

Although YM155 has been administered to cancer patients in several Phase I and II clinical trials, poor effectiveness limited the further application of YM155 as a single-agent therapy. Currently, two clinical trials are studying the combination effect of YM155 with Rituximab or Paclitaxel plus Carboplatin in lymphoma and non-small cell lung cancer, respectively. To explore more effective combinations with YM155, the inventors screened additional compounds for their association with ovarian cancer. The downstream target of p53, miR-34c, has been reported to be a tumor suppressor miRNA (Corney et al., 2007; He et al., 2007). In the Dicer/Pten serous ovarian cancer model (Kim et al., 2012), miR-34c was the only miRNA to suppress mouse and human ovarian cancer cell viability. When the inventors queried the CMap database (Lamb et al., 2006) with the miR-34c gene expression data to predict therapeutic responses of serous carcinomas to specific pharmacologic agents for ovarian cancer therapy, parthenolide (Mathema et al., 2011) was one of the top hits. Encouragingly, a synergy between YM155 and parthenolide was identified in established ovarian cancer cell lines (FIG. 19A, B) and a pancreatic cancer cell line (FIG. 19C). When levels of YM155 or parthenolide alone inhibited ˜50% of cancer cell viability, the combination of these two compounds inhibited nearly >90% of cancer cells. The findings indicate synergy of these two compounds.

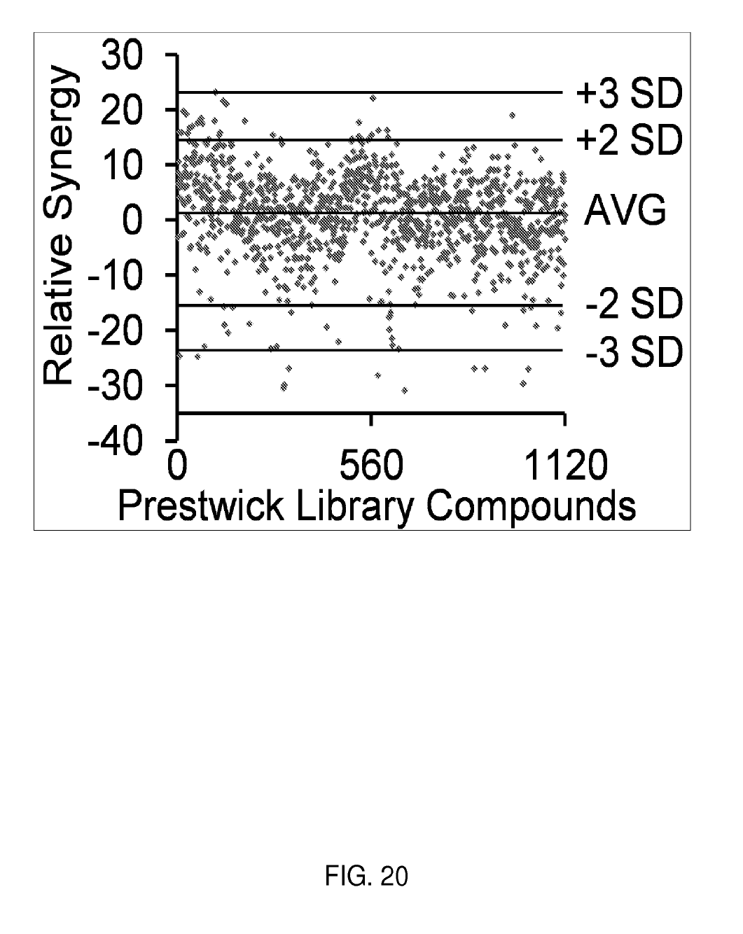

To identify additional small molecules that synergize with YM155, a small library screen was performed. Using this screen, the response of OVCAR8 cells to 1120 small molecules from the Prestwick Chemical Library (90% off-patent drugs and 10% bioactive compounds) was probed. OVCAR8 cells (500 cells/well of 384 plate) were seeded on day 1, treated with or without YM155 (3 nM) in the presence of the small molecule compounds (4 ug/ml) on day 2, and analyzed for cell death on day 4. There were 22 structurally and pathway diverse compounds that fall more than 2 S.D. above the mean for their “synergistic” effects with YM155 compared to their effects alone (FIG. 20). Also, there were 35 compounds that “protected” the OVCAR8 cells from the YM155 treatment (more than 2 S.D. below the mean). These data demonstrate the feasibility of this high-throughput screening approach to identify additional small molecules/drugs that synergize with YM155.

Twenty-one compounds reduced OVCAR8 cell survival to 2-20% by themselves in a low micromolar range and were not evaluated for synergy. This list includes expected cytotoxic drugs including cardiac glycosides (e.g., Digoxin), tubulin binders (e.g., colchicine), and topoisomerase inhibitors (e.g., camptothecin), further confirming the effectiveness of the screen. However, there were novel small molecules that caused cytotoxic effects. For example, Piperlongumine, a natural product derived from the fruit of the long pepper (Piper longum) reduced cell survival to 10% of the control at a concentration of 6.3 mM. Piperlongumine was identified recently to kill multiple cancer cells (including pancreatic cancer lines) by increasing the level of ROS (Raj et al., 2011) in a similar concentration range as the inventors discovered. These data further indicate that YM155 and parthenolide synergize via the ability of YM155 to target the HSP72 and the ER stress pathway and parthenolide to increase ROS and contribute to cellular stress.

### REFERENCES

All patents and publications mentioned in the specification are indicative of the levels of those skilled in the art to which the invention pertains. All patents and publications are herein incorporated by reference in their entirety to the same extent as if each individual publication was specifically and individually indicated to be incorporated by reference

### PUBLICATIONS

A. Jemal, R. Siegel, J. Xu, E. Ward, C A Cancer J Clin 60, 277 (September-October).
A. Mirza et al., Oncogene 21, 2613 (Apr. 18, 2002).
A. Ventura, T. Jacks, Cell 136, 586 (Feb. 20, 2009).
A. A. Ahmed et al., J Pathol 221, 49 (May, 2010).
A. K. Nagaraja et al., Mol Endocrinol 22, 2336 (October, 2008).
A. K. Nagaraja et al., Mol Endocrinol 24, 447 (February, 2010).
Aksamitiene E, Kholodenko B N, Kolch W, Hoek J B, Kiyatkin A. PI3K/Akt-sensitive MEK-independent compensatory circuit of ERK activation in E R-positive PI3K-mutant T47D breast cancer cells. Cellular signalling. 2010; 22(9):1369-78.
Altieri D C. New wirings in the survivin networks. Oncogene. 2008; 27(48):6276-84.
Attardi L D, Jacks T. The role of p53 in tumour suppression: lessons from mouse models. Cell Mol Life Sci. 1999; 55(1):48-63.
Bast R C, Jr., Hennessy B, Mills G B. The biology of ovarian cancer: new opportunities for translation. Nature reviews. 2009; 9(6):415-28.
Bast R C, Jr., Xu F J, Yu Y H, Barnhill S, Zhang Z, Mills G B. C A 125: the past and the future. The International journal of biological markers. 1998; 13(4):179-87.
Bau S, Schracke N, Kranzle M, Wu H, Stahler P F, Hoheisel J D, et al. Targeted next-generation sequencing by specific capture of multiple genomic loci using low-volume microfluidic DNA arrays. Anal Bioanal Chem. 2009; 393(1):171-5.
Bommer G T, Gerin I, Feng Y, Kaczorowski A J, Kuick R, Love R E, et al. p53-mediated activation of miRNA34 candidate tumor-suppressor genes. Curr Biol. 2007; 17(15):1298-307.
Buchold G M, Coarfa C, Kim J, Milosavljevic A, Gunaratne P H, Matzuk M M. Analysis of microRNA expression in the prepubertal testis. PloS one. 2010; 5(12):e15317.
Bunney T D, Katan M. Phosphoinositide signalling in cancer: beyond PI3K and PTEN. Nature reviews. 2010; 10(5):342-52.
C. C. Huang, S. P. Jamin, G. D. Orvis, N. A. Agango, R. R. Behringer, Science, submitted, (2011).
C. J. Creighton et al., Cancer Res 70, 1906 (Mar. 1, 2010).
Chang T C, Wentzel E A, Kent O A, Ramachandran K, Mullendore M, Lee K H, et al. Transactivation of miR-34a by p53 broadly influences gene expression and promotes apoptosis. Mol. Cell. 2007; 26(5):745-52.
Cheson, B. D., Bartlett, N. L., Vose, J. M., Lopez-Hernandez, A., Seiz, A. L., Keating, A. T., and Shamsili, S. (2011). A phase II study of the survivin suppressant YM155 in patients with refractory diffuse large B-cell lymphoma. Cancer.
Cho K R, Shih Ie M. Ovarian cancer. Annual review of pathology. 2009; 4:287-313.
Cohen C, Lohmann C M, Cotsonis G, Lawson D, Santoianni R. Survivin expression in ovarian carcinoma: correlation with apoptotic markers and prognosis. Mod Pathol. 2003; 16(6):574-83.
Comprehensive genomic characterization defines human glioblastoma genes and core pathways. Nature. 2008; 455(7216):1061-8.
Cooper G M, Shendure J. Needles in stacks of needles: finding disease-causal variants in a wealth of genomic data. Nat Rev Genet. 2011; 12(9):628-40.
Corney D C, Flesken-Nikitin A, Godwin A K, Wang W, Nikitin A Y. MicroRNA-34b and MicroRNA-34c are targets of p53 and cooperate in control of cell proliferation and adhesion-independent growth. Cancer research. 2007; 67(18):8433-8.
Corney D C, Hwang C I, Matoso A, Vogt M, Flesken-Nikitin A, Godwin A K, et al. Frequent downregulation of miR-34 family in human ovarian cancers. Clin Cancer Res. 2010; 16(4):1119-28.
Corney, D. C., Flesken-Nikitin, A., Godwin, A. K., Wang, W., and Nikitin, A. Y. (2007). MicroRNA-34b and MicroRNA-34c are targets of p53 and cooperate in control of cell proliferation and adhesion-independent growth. Cancer research 67, 8433-8438.
Creighton C J, Benham A L, Zhu H, Khan M F, Reid J G, Nagaraja A K, et al. Discovery of Novel MicroRNAs in Female Reproductive Tract Using Next Generation Sequencing. PloS one. 2010; 5(3):e9637.
Creighton C J, Fountain M D, Yu Z, Nagaraja A K, Zhu H, Khan M, et al. Molecular profiling uncovers a p53-associated role for microRNA-31 in inhibiting the proliferation of serous ovarian carcinomas and other cancers. Cancer research. 2010; 70(5):1906-15.
Creighton C J, Nagaraja A K, Hanash S M, Matzuk M M, Gunaratne P H. A bioinformatics tool for linking gene expression profiling results with public databases of microRNA target predictions. RNA 2008; 14(11):2290-6.
Crum C P, Drapkin R, Miron A, Ince T A, Muto M, Kindelberger D W, et al. The distal fallopian tube: a new model for pelvic serous carcinogenesis. Current opinion in obstetrics & gynecology. 2007; 19(1):3-9.
D. Milliken, C. Scotton, S. Raju, F. Balkwill, J. Wilson, Clin Cancer Res 8, 1108 (April, 2002).
D. C. Altieri, Oncogene 27, 6276 (Oct. 20, 2008).
Donehower L A. The p53-deficient mouse: a model for basic and applied cancer studies. Seminars in cancer biology. 1996; 7(5):269-78.
Du T, Zamore P D. microPrimer: the biogenesis and function of microRNA. Development 2005; 132(21):4645-52.
Edson M A, Nagaraja A K, Matzuk M M. The mammalian ovary from genesis to revelation. Endocrine reviews. 2009; 30(6):624-712.
Esquela-Kerscher A, Slack F J. Oncomirs—microRNAs with a role in cancer. Nature reviews. 2006; 6(4):259-69.
Fan H Y, Liu Z, Paquet M, Wang J, Lydon J P, DeMayo F J, et al. Cell type-specific targeted mutations of Kras and Pten document proliferation arrest in granulosa cells versus oncogenic insult to ovarian surface epithelial cells. Cancer research. 2009; 69(16):6463-72.
Ferrer-Costa C, Gelpi J L, Zamakola L, Parraga I, de la Cruz X, Orozco M. PMUT: a web-based tool for the annotation of pathological mutations on proteins. Bioinformatics 2005; 21(14):3176-8.
G. Pampalakis, E. P. Diamandis, D. Katsaros, G. Sotiropoulou, Clin Biochem 43, 324 (February, 2009).
G. A. Calin et al., Proc Natl Acad Sci USA 101, 2999 (Mar. 2, 2004).
Gabai, V. L., Yaglom, J. A., Waldman, T., and Sherman, M. Y. (2009). Heat shock protein Hsp72 controls oncogene-induced senescence pathways in cancer cells. Mol Cell Biol 29, 559-569.
Gamido, C., Schmitt, E., Cande, C., Vahsen, N., Parcellier, A., and Kroemer, G. (2003). HSP27 and HSP70: potentially oncogenic apoptosis inhibitors. Cell cycle (Georgetown, Tex. 2, 579-584.
Giaccone, G., Zatloukal, P., Roubec, J., Floor, K., Musil, J., Kuta, M., van Klayeren, R. J., Chaudhary, S., Gunther, A., and Shamsili, S. (2009). Multicenter phase II trial of YM155, a small-molecule suppressor of survivin, in patients with advanced, refractory, non-small-cell lung cancer. J Clin Oncol 27, 4481-4486.
Giles F J, Cortes J E, Kantarjian H M. Targeting the kinase activity of the BCR-ABL fusion protein in patients with chronic myeloid leukemia. Current molecular medicine. 2005; 5(7):615-23.
Glaros, T. G., Stockwin, L. H., Mullendore, M. E., Smith, B., Morrison, B. L., and Newton, D. L. (2012). The “survivin suppressants” NSC 80467 and YM155 induce a DNA damage response. Cancer chemotherapy and pharmacology.
Gupta, S., Deepti, A., Deegan, S., Lisbona, F., Hetz, C., and Samali, A. (2010). HSP72 protects cells from E R stress-induced apoptosis via enhancement of IRE1alpha-XBP1 signaling through a physical interaction. PLoS biology 8, e1000410.
Guzman, M. L., Rossi, R. M., Karnischky, L., Li, X., Peterson, D. R., Howard, D. S., and Jordan, C. T. (2005). The sesquiterpene lactone parthenolide induces apoptosis of human acute myelogenous leukemia stem and progenitor cells. Blood 105, 4163-4169.
H. B. Salvesen et al., Proc Natl Acad Sci USA 106, 4834 (Mar. 24, 2009).
H. Y. Fan et al., Cancer Res 69, 6463 (Aug. 15, 2009).
Hahm E R, Moura M B, Kelley E E, Van Houten B, Shiva S, Singh S V. Withaferin A-induced apoptosis in human breast cancer cells is mediated by reactive oxygen species. PloS one. 2011; 6(8):e23354.
Hattori Y. Development of non-viral vector for cancer gene therapy. Yakugaku Zasshi. 2010; 130(7):917-23.
Haupt Y, Maya R, Kazaz A, Oren M. Mdm2 promotes the rapid degradation of p53. Nature. 1997; 387(6630):296-9.
Hawkins S M, Creighton C J, Han D Y, Zariff A, Anderson M L, Gunaratne P H, et al. Functional microRNA involved in endometriosis. Molecular endocrinology 2011; 25(5):821-32.
He, L., He, X., Lim, L. P., de Stanchina, E., Xuan, Z., Liang, Y., Xue, W., Zender, L., Magnus, J., Ridzon, D., et al. (2007). A microRNA component of the p53 tumour suppressor network. Nature. 2007; 447(7148):1130-4.
Honda R, Tanaka H, Yasuda H. Oncoprotein MDM2 is a ubiquitin ligase E3 for tumor suppressor p53. FEBS letters. 1997; 420(1):25-7.
Hu F, Gartenhaus R B, Eichberg D, Liu Z, Fang H B, Rapoport A P. PBK/TOPK interacts with the DBD domain of tumor suppressor p53 and modulates expression of transcriptional targets including p21. Oncogene. 2010; 29(40):5464-74.
I. Meinhold-Heerlein et al., Clin Cancer Res 13, 458 (Jan. 15, 2007).
Integrated genomic analyses of ovarian carcinoma. Nature. 2011; 474(7353):609-15.
J. D. Seidman et al., Int J Gynecol Pathol 23, 41 (January, 2004).
J. D. Storey, R. Tibshirani, Proc Natl Acad Sci USA 100, 9440 (Aug. 5, 2003).
Jemal A, Siegel R, Ward E, Hao Y, Xu J, Thun M J. Cancer statistics, 2009. C A: a cancer journal for clinicians. 2009; 59(4):225-49.
Jones S, Wang T L, Shih Ie M, Mao T L, Nakayama K, Roden R, et al. Frequent mutations of chromatin remodeling gene ARID1A in ovarian clear cell carcinoma. Science 2010; 330(6001):228-31.
K. R. Cho, M. Shih Ie, Annu Rev Pathol 4, 287 (2009).
K. R. Kalli et al., Gynecol Oncol 108, 619 (March, 2008).
Kalli K R, Oberg A L, Keeney G L, Christianson T J, Low P S, Knutson K L, et al. Folate receptor alpha as a tumor target in epithelial ovarian cancer. Gynecologic oncology. 2008; 108(3):619-26.
Kim, J., Coffey, D. M., Creighton, C. J., Yu, Z., Hawkins, S. M., and Matzuk, M. M. (2012). High-grade serous ovarian cancer arises from fallopian tube in a mouse model. Proc Natl Acad Sci USA 109, 3921-3926.
Kindelberger D W, Lee Y, Miron A, Hirsch M S, Feltmate C, Medeiros F, et al. Intraepithelial carcinoma of the fimbria and pelvic serous carcinoma: Evidence for a causal relationship. The American journal of surgical pathology. 2007; 31(2):161-9.
Koonings P P, Campbell K, Mishell D R, Jr., Grimes D A. Relative frequency of primary ovarian neoplasms: a 10-year review. Obstetrics and gynecology. 1989; 74(6):921-6.
Kumar M S, Lu J, Mercer K L, Golub T R, Jacks T. Impaired microRNA processing enhances cellular transformation and tumorigenesis. Nat. Genet. 2007; 39(5):673-7.
L. Zhang et al., Proc Natl Acad Sci USA 105, 7004 (May 13, 2008).
Lamb, J., Crawford, E. D., Peck, D., Modell, J. W., Blat, I. C., Wrobel, M. J., Lerner, J., Brunet, J. P., Subramanian, A., Ross, K. N., et al. (2006). The Connectivity Map: using gene-expression signatures to connect small molecules, genes, and disease. Science (New York, N. Y. 2006; 313(5795):1929-35.
Ledermann J A, Raja F A. Targeted trials in ovarian cancer. Gynecologic oncology. 2010; 119(1):151-6.
Lee C H, Subramanian S, Beck A H, Espinosa I, Senz J, Zhu S X, et al. MicroRNA profiling of BRC A1/2 mutation-carrying and non-mutation-carrying high-grade serous carcinomas of ovary. PloS one. 2009; 4(10):e7314.
Lewis, K. D., Samlowski, W., Ward, J., Catlett, J., Cranmer, L., Kirkwood, J., Lawson, D., Whitman, E., and Gonzalez, R. (2011). A multi-center phase II evaluation of the small molecule survivin suppressor YM155 in patients with unresectable stage III or I V melanoma. Investigational new drugs 29, 161-166.
Li H, Durbin R. Fast and accurate short read alignment with Burrows-Wheeler transform. Bioinformatics 2009; 25(14):1754-60.
Li H, Handsaker B, Wysoker A, Fennell T, Ruan J, Homer N, et al. The Sequence Alignment/Map format and SAMtools. Bioinformatics 2009; 25(16):2078-9.
Lundberg, B. B. (1997). Ether lipids enhance the cytotoxic effect of teniposide and paclitaxel in liposomes against leukaemic cells in culture. Anti-cancer drug design 12, 503-513.
M. S. Kumar, J. Lu, K. L. Mercer, T. R. Golub, T. Jacks, Nat Genet. 39, 673 (May, 2007).
M. V. Iorio et al., Cancer Res 67, 8699 (Sep. 15, 2007).
Ma L, Buchold G M, Greenbaum M P, Roy A, Bums K H, Zhu H, et al. GASZ Is Essential for Male Meiosis and Suppression of Retrotransposon Expression in the Male Germline. PLoS Genet. 2009; 5(9):e1000635.
Marks J R, Davidoff A M, Kerns B J, Humphrey P A, Pence J C, Dodge R K, et al. Overexpression and mutation of p53 in epithelial ovarian cancer. Cancer research. 1991; 51(11):2979-84.
Marquez R T, Baggerly K A, Patterson A P, Liu J, Broaddus R, Frumovitz M, et al. Patterns of gene expression in different histotypes of epithelial ovarian cancer correlate with those in normal fallopian tube, endometrium, and colon. Clin Cancer Res. 2005; 11(17):6116-26.
Martinelli S, Kostylina G, Niggli V, Baumann C, Fey M F, Wendel H G, et al. Targeting survivin via PI3K but not c-akt/PKB by anticancer drugs in immature neutrophils. Oncogene. 2006; 25(52):6915-23.
Mathema, V. B., Koh, Y. S., Thakuri, B. C., and Sillanpaa, M. (2011). Parthenolide, a Sesquiterpene Lactone, Expresses Multiple Anti-cancer and Anti-inflammatory Activities. Inflammation.
Matzuk M M, Finegold M J, Su J G, Hsuch A J, Bradley A. Alpha-inhibin is a tumour-suppressor gene with gonadal specificity in mice. Nature. 1992; 360(6402):313-9.
Medeiros F, Muto M G, Lee Y, Elvin J A, Callahan M J, Feltmate C, et al. The tubal fimbria is a preferred site for early adenocarcinoma in women with familial ovarian cancer syndrome. The American journal of surgical pathology. 2006; 30(2):230-6.
Meinhold-Heerlein I, Bauerschlag D, Zhou Y, Sapinoso L M, Ching K,
Frierson H, Jr., et al. An integrated clinical-genomics approach identifies a candidate multi-analyte blood test for serous ovarian carcinoma. Clin Cancer Res. 2007; 13(2 Pt 1):458-66.
Merritt W M, Lin Y G, Han L Y, Kamat A A, Spannuth W A, Schmandt R, et al. Dicer, Drosha, and outcomes in patients with ovarian cancer. The New England journal of medicine. 2008; 359(25):2641-50.
Meyerson M, Gabriel S, Getz G. Advances in understanding cancer genomes through second-generation sequencing. Nat Rev Genet. 2010; 11(10):685-96.
Milliken D, Scotton C, Raju S, Balkwill F, Wilson J. Analysis of chemokines and chemokine receptor expression in ovarian cancer ascites. Clin Cancer Res. 2002; 8(4):1108-14.
Mitelman F, Johansson B, Mertens F. The impact of translocations and gene fusions on cancer causation. Nature reviews. 2007; 7(4):233-45.
Modesitt S C, Parsons S J. In vitro and in vivo histone deacetylase inhibitor therapy with vorinostat and paclitaxel in ovarian cancer models: does timing matter? Gynecologic oncology. 2010; 119(2):351-7.
Mooney S. Bioinformatics approaches and resources for single nucleotide polymorphism functional analysis. Briefings in bioinformatics. 2005; 6(1):44-56.
Mootha V K, Lindgren C M, Eriksson K F, Subramanian A, Sihag S, Lehar J, et al. PGC-1alpha-responsive genes involved in oxidative phosphorylation are coordinately downregulated in human diabetes. Nat. Genet. 2003; 34(3):267-73.
Morimoto, R. I., Kline, M. P., Bimston, D. N., and Cotto, J. J. (1997). The heat-shock response: regulation and function of heat-shock proteins and molecular chaperones. Essays in biochemistry 32, 17-29.
N. A. Arango et al., Mol Reprod Dev 75, 1154 (July, 2008).
Nagaraja A K, Andreu-Vieyra C, Franco H L, Ma L, Chen R, Han D Y, et al. Deletion of Dicer in somatic cells of the female reproductive tract causes sterility. Molecular Endocrinology. 2008; 22(10):2336-52.
Nagaraja A K, Creighton C J, Yu Z, Zhu H, Gunaratne P H, Reid J G, et al. A link between mir-100 and FRA P1/mT OR in clear cell ovarian cancer. Molecular endocrinology 2010; 24(2):447-63.
Nakahara, T., Kita, A., Yamanaka, K., Mori, M., Amino, N., Takeuchi, M., Tominaga, F., Kinoyama, I., Matsuhisa, A., Kudou, M., et al. (2011). Broad spectrum and potent antitumor activities of YM155, a novel small-molecule survivin suppressant, in a wide variety of human cancer cell lines and xenograft models. Cancer science.102(3):614-21.
Nakahara, T., Takeuchi, M., Kinoyama, I., Minematsu, T., Shirasuna, K., Matsuhisa, A., Kita, A., Tominaga, F., Yamanaka, K., Kudoh, M., et al. (2007). YM155, a novel small-molecule survivin suppressant, induces regression of established human hormone-refractory prostate tumor xenografts. Cancer research 67, 8014-8021.
Nakamura, N., Yamauchi, T., Hiramoto, M., Yuri, M., Naito, M., Takeuchi, M., Yamanaka, K., Kita, A., Nakahara, T., Kinoyama, I., et al. (2012). ILF3/N F110 is a target of YM155, a suppressant of survivin. Mol Cell Proteomics.
Ng P C, Henikoff S. Predicting deleterious amino acid substitutions. Genome research. 2001; 11(5):863-74.
Nikolaev S I, Iseli C, Sharp A J, Robyr D, Rougemont J, Gehrig C, et al. Detection of genomic variation by selection of a 9 mb DNA region and high throughput sequencing. PloS one. 2009; 4(8):e6659.
Okamoto A, Sameshima Y, Yokoyama S, Terashima Y, Sugimura T, Terada M, et al. Frequent allelic losses and mutations of the p53 gene in human ovarian cancer. Cancer research. 1991; 51(19):5171-6.
Okou D T, Locke A E, Steinberg K M, Hagen K, Athri P, Shetty A C, et al. Combining microarray-based genomic selection (MGS) with the Illumina Genome Analyzer platform to sequence diploid target regions. Ann Hum Genet. 2009; 73(Pt 5):502-13.
Olive K P, Tuveson D A, Ruhe Z C, Yin B, Willis N A, Bronson R T, et al. Mutant p53 gain of function in two mouse models of Li-Fraumeni syndrome. Cell. 2004; 119(6):847-60.
Parsons D W, Jones S, Zhang X, Lin J C, Leary R J, Angenendt P, et al. An integrated genomic analysis of human glioblastoma multiforme. Science 2008; 321(5897):1807-12.
Peng D J, Wang J, Zhou J Y, Wu G S. Role of the Akt/mTOR survival pathway in cisplatin resistance in ovarian cancer cells. Biochem Biophys Res Commun 2010; 394(3):600-5.
Polyak K, Weinberg R A. Transitions between epithelial and mesenchymal states: acquisition of malignant and stem cell traits. Nature reviews. 2009; 9(4):265-73.
R. C. Bast, Jr., B. Hennessy, G. B. Mills, Nat Rev Cancer 9, 415 (June, 2009).
R. J. Flavin et al., Mod Pathol 21, 676 (June, 2008).
R. J. Kurman, M. Shih Ie, Am J Surg Pathol 34, 433 (March, 2010).
Raj, L., Ide, T., Gurkar, A. U., Foley, M., Schenone, M., Li, X., Tolliday, N. J., Golub, T. R., Carr, S. A., Shamji, A. F., et al. (2011). Selective killing of cancer cells by a small molecule targeting the stress response to ROS. Nature 475, 231-234.
Raver-Shapira N, Marciano E, Meiri E, Spector Y, Rosenfeld N, Moskovits N, et al. Transcriptional activation of miR-34a contributes to p53-mediated apoptosis. Mol. Cell. 2007; 26(5):731-43.
Reid J G, Nagaraja A K, Lynn F C, Drabek R B, Muzny D M, Shaw C A, et al. Mouse let-7 miRNA populations exhibit RNA editing that is constrained in the 5′-seed/cleavage/anchor regions and stabilize predicted mmu-let-7a:mRNA duplexes. Genome research. 2008; 18(10):1571-81.
S. Martinelli et al., Oncogene 25, 6915 (Nov. 2, 2006).
S. Salvador et al., Int J Gynecol Cancer 19, 58 (January, 2009).
Sah, N. K., Khan, Z., Khan, G. J., and Bisen, P. S. (2006). Structural, functional and therapeutic biology of survivin. Cancer letters 244, 164-171.
Salvesen H B, Carter S L, Mannelqvist M, Dutt A, Getz G, Stefansson I M, et al. Integrated genomic profiling of endometrial carcinoma associates aggressive tumors with indicators of PI3 kinase activation. Proc Natl Acad Sci USA. 2009; 106(12):4834-9.
Samali, A., and Orrenius, S. (1998). Heat shock proteins: regulators of stress response and apoptosis. Cell stress & chaperones 3, 228-236.
Satoh, T., Okamoto, I., Miyazaki, M., Morinaga, R., Tsuya, A., Hasegawa, Y., Terashima, M., Ueda, S., Fukuoka, M., Ariyoshi, Y., et al. (2009). Phase I study of YM155, a novel survivin suppressant, in patients with advanced solid tumors. Clin Cancer Res 15, 3872-3880.
Seidman J D, Zhao P, Yemelyanova A. “Primary peritoneal” high-grade serous carcinoma is very likely metastatic from serous tubal intraepithelial carcinoma: Assessing the new paradigm of ovarian and pelvic serous carcinogenesis and its implications for screening for ovarian cancer. Gynecologic oncology. 2010.
Shah S P, Kobel M, Senz J, Morin R D, Clarke B A, Wiegand K C, et al. Mutation of FOXL2 in granulosa-cell tumors of the ovary. The New England journal of medicine. 2009; 360(26):2719-29.
Shanmugam, R., Kusumanchi, P., Cheng, L., Crooks, P., Neelakantan, S., Matthews, W., Nakshatri, H., and Sweeney, C. J. (2010). A water-soluble parthenolide analogue suppresses in vivo prostate cancer growth by targeting NFkappaB and generating reactive oxygen species. The Prostate 70, 1074-1086.
Shaw A T, Winslow M M, Magendantz M, Ouyang C, Dowdle J, Subramanian A, et al. Selective killing of K-ras mutant cancer cells by small molecule inducers of oxidative stress. Proc Natl Acad Sci USA. 2011; 108(21):8773-8.
Siepel A, Pollard K S, Haussler D, editors. New methods for detecting lineage-specific selection. Proceedings of the 10th International Conference of Research in Computational Molecular Biology; 2006; RECOMB.
Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005; 102(43):15545-50.
Sui L, Dong Y, Watanabe Y, Yamaguchi F, Sugimoto K, Tokuda M. Alteration and clinical relevance of PTEN expression and its correlation with survivin expression in epithelial ovarian tumors. Oncology reports. 2006; 15(4):773-8.
Summerer D, Schracke N, Wu H, Cheng Y, Bau S, Stahler C F, et al. Targeted high throughput sequencing of a cancer-related exome subset by specific sequence capture with a fully automated microarray platform. Genomics. 2010; 95(4):241-6.
Summerer D, Wu H, Haase B, Cheng Y, Schracke N, Stahler C F, et al. Microarray-based multicycle-enrichment of genomic subsets for targeted next-generation sequencing. Genome research. 2009; 19(9):1616-21.
Sunyaev S, Ramensky V, Bork P. Towards a structural basis of human non-synonymous single nucleotide polymorphisms. Trends Genet. 2000; 16(5):198-200.
Sunyaev S, Ramensky V, Koch I, Lathe W, 3rd, Kondrashov A S, Bork P. Prediction of deleterious human alleles. Human molecular genetics. 2001; 10(6):591-7.
T. D. Bunney, M. Katan, Nat Rev Cancer 10, 342 (May, 2010).
Takai N, Ueda T, Nishida M, Nasu K, Narahara H. A novel histone deacetylase inhibitor, Scriptaid, induces growth inhibition, cell cycle arrest and apoptosis in human endometrial cancer and ovarian cancer cells. International journal of molecular medicine. 2006; 17(2):323-9.
Tarasov V, Jung P, Verdoodt B, Lodygin D, Epanchintsev A, Menssen A, et al. Differential regulation of microRNAs by p53 revealed by massively parallel sequencing: miR-34a is a p53 target that induces apoptosis and G1-arrest. Cell cycle (Georgetown, Tex. 2007; 6(13):1586-93.
Tariq M A, Kim H J, Jejelowo O, Pourmand N. Whole-transcriptome RNAseq analysis from minute amount of total RNA. Nucleic acids research. 2011.
Tavaria, M., Gabriele, T., Kola, I., and Anderson, R. L. (1996). A hitchhiker's guide to the human Hsp70 family. Cell stress & chaperones 1, 23-28.
Tolcher, A. W., Mita, A., Lewis, L. D., Garrett, C. R., Till, E., Daud, A. I., Patnaik, A., Papadopoulos, K., Takimoto, C., Bartels, P., et al. (2008). Phase I and pharmacokinetic study of YM155, a small-molecule inhibitor of survivin. J Clin Oncol 26, 5198-5203.
Tolcher, A. W., Quinn, D. I., Ferrari, A., Ahmann, F., Giaccone, G., Drake, T., Keating, A., and de Bono, J. S. (2012). A phase II study of YM155, a novel small-molecule suppressor of survivin, in castration-resistant taxane-pretreated prostate cancer. Ann Oncol 23, 968-973.
Tsukishiro S, Suzumori N, Nishikawa H, Arakawa A, Suzumori K. Elevated serum RANTES levels in patients with ovarian cancer correlate with the extent of the disorder. Gynecologic oncology. 2006; 102(3):542-5.
U. W. Rothenpieler, G. R. Dressler, Development 119, 711 (November, 1993).
Varley J M. Germline T P53 mutations and Li-Fraumeni syndrome. Human mutation. 2003; 21(3):313-20.
W. H. Hoffman, S. Biade, J. T. Zilfou, J. Chen, M. Murphy, J Biol Chem 277, 3247 (Feb. 1, 2002).
W. M. Merritt et al., N Engl J Med 359, 2641 (Dec. 18, 2008).
Wang, G., Yang, Z. Q., and Zhang, K. (2010). Endoplasmic reticulum stress response in cancer: molecular mechanism and therapeutic potential. American journal of translational research 2, 65-74.
Wang, J., Tian S, Petros R A, Napier M E, Desimone J M. The complex role of multivalency in nanoparticles targeting the transferrin receptor for cancer therapies. Journal of the American Chemical Society. 2010; 132(32):11306-13.
Werner M E, Karve S, Sukumar R, Cummings N D, Copp J A, Chen R C, et al. Folate-targeted nanoparticle delivery of chemo- and radiotherapeutics for the treatment of ovarian cancer peritoneal metastasis. Biomaterials. 2011; 32(33):8548-54.
Wiegand K C, Shah S P, Al-Agha O M, Zhao Y, Tse K, Zeng T, et al. ARID1A mutations in endometriosis-associated ovarian carcinomas. The New England journal of medicine. 2010; 363(16):1532-43.
Yaglom, J. A., Gabai, V. L., and Sherman, M. Y. (2007). High levels of heat shock protein Hsp72 in cancer cells suppress default senescence pathways. Cancer research 67, 2373-2381.
Yang, Y. I., Kim, J. H., Lee, K. T., and Choi, J. H. (2011). Costunolide induces apoptosis in platinum-resistant human ovarian cancer cells by generating reactive oxygen species. Gynecologic oncology 123, 588-596.
Yue P, Melamud E, Moult J. S NPs3D: candidate gene and SNP selection for association studies. BMC bioinformatics. 2006; 7:166.
Zaffaroni N, Pennati M, Colella G, Perego P, Supino R, Gatti L, et al. Expression of the anti-apoptotic gene survivin correlates with taxol resistance in human ovarian cancer. Cell Mol Life Sci. 2002; 59(8):1406-12.
Zhao N, Fogg J M, Zechiedrich L, Zu Y. Transfection of shRNA-encoding Minivector DNA of a few hundred base pairs to regulate gene expression in lymphoma cells. Gene therapy. 2011; 18(3):220-4.
Zhu Y, Hoffman A, Wu X, Zhang H, Zhang Y, Leaderer D, et al. Correlating observed odds ratios from lung cancer case-control studies to SNP functional scores predicted by bioinformatic tools. Mutation research. 2008; 639(1-2):80-8.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of treating serous ovarian cancer in an individual, comprising the step of delivering to the individual a therapeutically effective amount of miR-34c or a miR-34c mimic.

2. The method of claim 1, wherein the individual is provided at least one other treatment for serous ovarian cancer.

3. The method of claim 2, wherein the other treatment comprises an inhibitor of survivin, an inhibitor or suppressor of HSPA1A and HSPA1B, a stimulator of endoplasmic reticulum stress, an mTOR inhibitor, withaferin A, parthenolide, piper longumine, vorinostat, scriptaid, camptothecin, small molecules that generate or enhance reactive oxygen species inhibitors of the anti-oxidant system, or a combination thereof.

4. The method of claim 3, wherein the inhibitor of survivin is YM155 or an siRNA or shRNA that targets survivin.

5. The method of claim 3, wherein the mTOR inhibitor is everolimus.

6. The method of claim 3, wherein the small molecule that generates or enhances reactive oxygen species is Motexafingadolium, Eleggclomol, parthenolide, piper longumine, dimethylamino-parthenolide, or costunolide.

7. The method of claim 3, wherein the inhibitor of the anti-oxidant system is Buthionine, sulphoximine, Imexon, Mangafodipir, 2-methoxyestradiol, or Tetrathiomolybdate.

8. The method of claim 3, wherein the inhibitor or suppressor of HSPA1A and HSPA1B is YM155 or an shRNA or siRNA that targets HSPA1A and HSPA1B.

9. The method of claim 3, wherein the combination therapy to treat ovarian cancer, pancreatic cancer, and other responsive cancers involves delivery of YM155 and parthenolide.

10. The method of claim 3, wherein the combination therapy to treat ovarian cancer, pancreatic cancer, and other responsive cancers involves delivery of a small molecule that enhances endoplasmic reticulum stress and generates or enhances reactive oxygen species or suppresses the anti-oxidant system.

11. The method of claim 10, wherein the small molecule that enhances endoplasmic reticulum stress is YM155.

12. The method of claim 10, wherein the small molecule that generates or enhances reactive oxygen species is parthenolide, dimethyl-parthenolide, or costunolide.

13. The method of claim 10, wherein the inhibitor of the anti-oxidant system is Buthionine, sulphoximine, Imexon, Mangafodipir, 2-methoxyestradiol, or Tetrathiomolybdate.

14. The method of claim 1, wherein the cancer is identified as having upregulation of a gene selected from the group consisting of Secreted phosphoprotein 1, Chemokine (C-X-C motif) ligand 9, Chemokine (C-X-C motif) ligand 10, CD72 antigen, Solute carrier family 15, member 3, CD84 antigen, Complement component 1qB, Plasminogen activator, urokinase, Lymphocyte antigen 86, Mucin 16 (CA125), Folate receptor 1, Solute carrier family 11, member 1, Solute carrier family 12, member 8, CD40 antigen, Immunoglobulin superfamily, member 9, Interleukin 10 receptor, alpha, Tumor necrosis factor receptor, member 12a, Apolipoprotein E, Toll-like receptor 7, Transmembrane protein 48, Interleukin 1 receptor, type II, Leukocyte-associated Ig-like receptor 1, Lymphocyte antigen 6 complex, locus E, A disintegrin and metallopeptidase domain 17, Pleiotrophin, CD83 antigen, Chemokine (C-C motif) ligand 8, Transmembrane channel-like gene family 6, Transmembrane protein 49, Endothelial cell-specific molecule 1, Anti-Mullerian hormone type 2 receptor, Midkine, Transmembrane protein 173, Tumor necrosis factor receptor, member 21, Complement factor B, Secretory carrier membrane protein 5, and a combination thereof.

15. The method of claim 1, wherein the cancer originates in the fallopian tube.

16. A method of determining whether an individual has serous ovarian cancer or is at risk for developing serous ovarian cancer, comprising the step of determining that when the expression of one or more of Secreted phosphoprotein 1, Chemokine (C-X-C motif) ligand 9, Chemokine (C-X-C motif) ligand 10, CD72 antigen, Solute carrier family 15, member 3, CD84 antigen, Complement component 1qB, Plasminogen activator, urokinase, Lymphocyte antigen 86, Mucin 16 (CA125), Folate receptor 1, Solute carrier family 11, member 1, Solute carrier family 12, member 8, CD40 antigen, Immunoglobulin superfamily, member 9, Interleukin 10 receptor, alpha, Tumor necrosis factor receptor, member 12a, Apolipoprotein E, Toll-like receptor 7, Transmembrane protein 48, Interleukin 1 receptor, type II, Leukocyte-associated Ig-like receptor 1, Lymphocyte antigen 6 complex, locus E, A disintegrin and metallopeptidase domain 17, Pleiotrophin, CD83 antigen, Chemokine (C-C motif) ligand 8, Transmembrane channel-like gene family 6, Transmembrane protein 49, Endothelial cell-specific molecule 1, Anti-Mullerian hormone type 2 receptor, Midkine, Transmembrane protein 173, Tumor necrosis factor receptor, member 21, Complement factor B, or Secretory carrier membrane protein 5 is upregulated in a sample from the individual, the individual has serous ovarian cancer or is at risk for having serous ovarian cancer.

17. A method of treating cancer in an individual, comprising the step of delivering a therapeutically effective amount of YM155 and parthenolide to the individual.

18. The method of claim 17, wherein the individual has ovarian cancer or pancreatic cancer.
